(19) United States
(12) Patent Application Publication
(10) Pub. No.: US 2024/0047081 A1
(43) Pub. Date: Feb. 8, 2024

(54) Designing Chemical or Genetic Perturbations using Artificial Intelligence

(71) Applicant: REGENTS OF THE UNIVERSITY OF MICHIGAN, Ann Arbor, MI (US)

(72) Inventors: Joshua Welch, Ann Arbor, MI (US); Hengshi Yu, Ann Arbor, MI (US)

(21) Appl. No.: 17/876,493

(22) Filed: Jul. 28, 2022

(51) Int. Cl. G16H 70/40; G06N 20/00

(52) U.S. Cl. CPC G16H 70/40; G06N 20/00

(57) ABSTRACT

The following relates generally to identifying perturbations (e.g., chemical perturbations, or genetic perturbations), drug treatments, and/or protein sequences. Some embodiments include a machine learning algorithm comprising a first network that converts perturbations into real-valued vector representations of the perturbations; a second network that converts cell states into real-valued vector representations of the cell states; and a third network that maps relationships between: (i) the real-valued vector representations of the perturbations, and (ii) the real-valued vector representations of the cell states. Some embodiments use the machine learning algorithm to identify a perturbation that will cause a starting cell state to transition to a target cell state by inputting the starting cell state and the target cell state into the machine learning algorithm.

### STATEMENT OF GOVERNMENT INTEREST

This invention was made with government support under HG010883 and HG011952 awarded by the National Institutes of Health. The government has certain rights in the invention.

### BACKGROUND

Recent developments in deep learning have enabled generation of novel and realistic images or sentences from low-dimensional representations. In this regard, several deep generative models have been developed to learn latent representations of cells and generate realistic high-dimensional single-cell data. However, these deep generative models generate data similar to that seen during training and have limited ability to predict gene expression of unseen cell states (e.g., these models are only able to predict gene expressions corresponding to what they have seen during training). In consequence, the applicability of these deep generative models is constrained for single-cell data, which usually have a relatively small set of observed conditions.

The systems and methods disclosed herein provide solutions to these problems and others.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one aspect, a computer-implemented method for identifying a perturbation may be provided. The method may include: (1) receiving, via one or more computer processors, an indication of a starting cell state; (2) receiving, via the one or more processors, an indication of a target cell state; and (3) identifying, via the one or more processors, a perturbation that will cause the starting cell state to transition to the target cell state by inputting the starting cell state and the target cell state into a trained machine learning algorithm.

In another aspect, a computer system for identifying a perturbation may be provided. The computer system may include one or more processors configured to: (1) receive an indication of a starting cell state; (2) receive an indication of a target cell state; and (3) identify a perturbation that will cause the starting cell state to transition to the target cell state by inputting the starting cell state and the target cell state into a trained machine learning algorithm.

In yet another aspect, a computing device for identifying a perturbation may be provided. The computing device may include: one or more processors; and one or more memories coupled to the one or more processors. The one or more memories including computer executable instructions stored therein that, when executed by the one or more processors, may cause the one or more processors to: (1) receive an indication of a starting cell state; (2) receive an indication of a target cell state; and (3) identify a perturbation that will cause the starting cell state to transition to the target cell state by inputting the starting cell state and the target cell state into a trained machine learning algorithm.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example architecture that may be used for identifying a perturbation that will cause a starting cell state to transition to a target cell state.

FIG. 2 illustrates an overview of an example of a chemical variational autoencoder architecture.

FIG. 3 illustrates an overview of an example of the disclosed PerturbNet architecture.

FIG. 4A shows the UMAP plots of the perturbation representations and the cellular representations of two drug treatments of S1628 and S1007 in the sci-Plex data.

FIG. 4B shows UMAP plots of perturbation representations and cellular representations of two drugs in Library of Integrated Network-Based Cellular Signatures (LINCS)-Drug data.

FIG. 5A shows R squared and Fréchet inception distance (FID) of k-nearest neighbors (KNN) model over random model for unseen and observed drug treatments of the sci-Plex data.

FIG. 5B shows R squared and FID of KNN model over random model for unseen and observed drug treatments of the LINCS-Drug data.

FIG. 6A shows R squared and FID of PerturbNet over baseline random model for unseen and observed drug treatments of the sci-Plex data.

FIG. 6B shows R squared and FID of PerturbNet over baseline random model for unseen and observed drug treatments of the LINCS-Drug data.

FIG. 7 shows R squared and FID of PerturbNet adjusted for cell state covariates over the unadjusted PerturbNet for 30 unseen and 158 observed drug treatments of the sci-Plex data.

FIG. 8A shows R squared and FID of KNN visualized by cell type.

FIG. 8B shows R squared and FID of KNN visualized by dose.

FIG. 8C shows R squared and FID of PerturbNet adjusted for covariates visualized by cell type.

FIG. 8D shows R squared and FID of PerturbNet adjusted for covariates visualized by cell dose.

FIG. 9A shows R squared and FID of PerturbNet over baseline KNN model for unseen and observed drug treatments of the sci-Plex data.

FIG. 9B shows R squared and FID of PerturbNet over baseline KNN model for unseen and observed drug treatments of the LINCS-Drug data.

FIG. 10A shows R squared and FID of PerturbNet adjusted for covariates over KNN for 30 unseen and 158 observed drug treatments, visualized by cell type.

FIG. 10B shows R squared and FID of PerturbNet adjusted for covariates over KNN for 30 unseen and 158 observed drug treatments, visualized by dose.

FIG. 11A shows R squared of predictions of cell type/treatment combinations using a latent space vector arithmetic, cell type translation, treatment translation and PerturbNet.

FIG. 11B shows R squared of predicted cell type/treatment combinations between PerturbNet and each of latent algorithm, cell type translation and treatment translation.

FIG. 12A illustrates an example UMAP plot of predicted MCF7-S1259 using a latent space vector algorithm.

FIG. 12B illustrates an example UMAP plot of predicted MCF7-S1259 using a cell type translation.

FIG. 12C illustrates an example UMAP plot of predicted MCF7-S1259 using a treatment translation.

FIG. 12D illustrates an example UMAP plot of predicted MCF7-S1259 using PerturbNet.

FIG. 13 shows example R squared and FID of KNN and PerturbNet with fine-tuned chemical variational autoencoder across different values for the 2000 unseen drug treatments of the LINCS-Drug data.

FIG. 14 shows example R squared and FID metrics of KNN and PerturbNet with fine-tuned chemical variational autoencoder of λ=1 over non-fine-tuned PerturbNet for 2000 unseen drug treatments of the LINCS-Drug data.

FIG. 15A shows example Uniform Manifold Approximation and Projection (UMAP) plots of perturbation representations and reconstructed cellular representations for S1628 and S1007 in the sci-Plex data.

FIG. 15B shows example UMAP plots of perturbation representations and reconstructed cellular representations as well as sampled cellular representations for S1628 and S1007 in the sci-Plex data.

FIG. 15C shows example UMAP plots of perturbation representations and reconstructed cellular representations for two drugs in the LINCS-Drug data.

FIG. 15D shows example UMAP plots of perturbation representations and reconstructed cellular representations as well as sampled cellular representations for two drugs in the LINCS-Drug data.

FIG. 16 illustrates an example genotype variational autoencoder (GenotypeVAE) framework.

FIG. 17 shows an example illustration of the Evolutionary Scale Modeling (ESM) architecture.

FIG. 18A shows example UMAP plots of perturbation representations and cellular representations of three pairs of genetic perturbations in the GI, LINCS-Gene and GSPS datasets.

FIG. 18B shows example UMAP plots of perturbation representations and cellular representations as well as reconstructed cellular representations of three pairs of genetic perturbations in the GI, LINCS-Gene and GSPS datasets

FIG. 19A shows an example representation of R squared and FID of KNN over the random model for 50 unseen and 180 observed genetic perturbations of the GI data.

FIG. 19B shows an example representation of R squared and FID PerturbNet over the random model for 50 unseen and 180 observed genetic perturbations of the GI data.

FIG. 20A shows an example representation of R squared and FID of KNN over the random model for 400 unseen and 3709 observed genetic perturbations of the LINCS-Gene data.

FIG. 20B shows an example representation of R squared and FID of PerturbNet over the random model for 400 unseen and 3709 observed genetic perturbations of the LINCS-Gene data.

FIG. 21A shows an example representation of R squared and FID of KNN over the random model for 802 unseen and 6859 observed genetic perturbations with more than 100 cells of the GSPS data.

FIG. 21B shows an example representation of R squared and FID of PerturbNet over the random model for 802 unseen and 6859 observed genetic perturbations with more than 100 cells of the GSPS data.

FIG. 22A shows an example representation of R squared and FID of PerturbNet over KNN for unseen and observed genetic perturbations of GI.

FIG. 22B shows an example representation of R squared and FID of PerturbNet over KNN for unseen and observed genetic perturbations of LINCS-Gene.

FIG. 22C shows an example representation of R squared and FID of PerturbNet over KNN for unseen and observed genetic perturbations of GSPS.

FIG. 22D shows an example representation of R squared and FID of PerturbNet over KNN for unseen and observed genetic perturbations of Ursu.

FIG. 23A shows an example representation of R squared and FID of KNN with fine-tuned chemical variational autoencoder across different values for 400 unseen and 3709 observed genetic perturbations of the LINCS-Gene data.

FIG. 23B shows an example representation of R squared and FID of PerturbNet with fine-tuned chemical variational autoencoder across different A values for 400 unseen and 3709 observed genetic perturbations of the LINCS-Gene data.

FIG. 24A shows an example representation of R squared and FID metrics of KNN with fine-tuned GenotypeVAE of λ=1 over non-fine-tuned PerturbNet for 400 unseen and 3709 observed genetic perturbations of the LINCS-Gene data.

FIG. 24B shows an example representation of R squared and FID metrics of PerturbNet with fine-tuned GenotypeVAE of λ=1 over non-fine-tuned PerturbNet for 400 unseen and 3709 observed genetic perturbations of the LINCS-Gene data.

FIG. 25 shows an example representation of UMAP plots of ESM representations and perturbation representations for protein perturbations of the Ursu data.

FIG. 26A UMAP plots of perturbation representations and cellular representations for two protein perturbations in the Ursu data.

FIG. 26B UMAP plots of perturbation representations and cellular representations as well as reconstructed cellular representations for two protein perturbations in the Ursu data.

FIG. 27A shows an example representation of R squared and FID of KNN over the random model for 16 unseen and 145 observed coding variants with more than 400 cells of the Ursu data.

FIG. 27B shows an example representation of R squared and FID of PerturbNet over the random model for 16 unseen and 145 observed coding variants with more than 400 cells of the Ursu data.

FIG. 28 illustrates an example overview of a translation optimization.

FIG. 29 shows example UMAP plots of latent values of cells treated by S1007, their translated latent values to treatment S1628, and latent values of real cells treated by S1628 in the sci-Plex data.

FIG. 30A shows an example scatter plot of normalized fitted W2 and normalized target W2 for 158 continuous optimal translations of the sci-Plex data.

FIG. 30B shows an example scatter plot of normalized fitted W2 and normalized target W2 for 200 continuous optimal translations of the LINCS-Drug data.

FIG. 30C shows an example scatter plot of fitted W2 percentile and target W2 percentile for 158 continuous optimal translations of the sci-Plex data.

FIG. 30D shows an example scatter plot of fitted W2 percentile and target W2 percentile for 200 continuous optimal translations of the LINCS-Drug data.

FIG. 30E shows an example histogram of the W2 distances between the target latent space of S1628 and the translated latent space from cells treated by S1172 to each of the 158 observed drug treatments, along with fitted W2, target W2 and their percentiles in the histogram.

FIG. 30F shows an example histogram of the W2 distances between the target latent space of a target drug treatment and the translated latent space from the cells treated by a starting drug treatment to each of the 2000 sampled observed drug treatments, along with fitted W2, target W2 and their percentiles in the histogram.

FIG. 31A shows an example scatter plot of normalized fitted W2 and normalized target W2 for 1580 discrete optimal translations.

FIG. 31B shows an example scatter plot of normalized fitted W2 and normalized target W2 for the 1580 discrete optimal translations by residual distance tertile.

FIG. 31C shows an example histogram of KNN indices of target perturbation to fitted perturbation for 1580 discrete optimal translations.

FIG. 31D shows an example histogram of percentiles of W2 distances between the latent spaces of the real cells treated by fitted perturbation and target perturbation in the distribution of the W2 distances between the latent spaces of the real cells treated by the target perturbation and other perturbations across the 1580 discrete optimal translations.

FIG. 31E shows an example histogram of the W2 distances between the target latent space of S1703 and the translated latent space from the cells treated by S1515 to each of the 158 observed drug treatments, along with fitted W2, target W2 and their percentiles in the histogram.

FIG. 32A shows an example scatter plot of normalized fitted W2 and normalized target W2 for 18,960 discrete optimal translations by cell type.

FIG. 32B shows an example scatter plot of normalized fitted W2 and normalized target W2 for 18,960 discrete optimal translations by dose.

FIG. 32C shows an example scatter plot of normalized fitted W2 and normalized target W2 for 18,960 discrete optimal translations by residual distance tertile.

FIG. 32D shows an example histogram of KNN indices of target perturbation to fitted perturbation for 18,960 discrete optimal translations.

FIG. 32E shows an example histogram of percentiles of W2 distances between the latent spaces of the real cells treated by fitted perturbation and target perturbation in the distribution of the W2 distances between the latent spaces of the real cells treated by the target perturbation and other perturbations across the 18,960 discrete optimal translations.

FIG. 32F shows an example histogram of the W2 distances between the target latent space of S1315 and the translated latent space from the cells treated by S1122 to each of the 158 observed drug treatments with cell type K562 and dose 10, along with fitted W2, target W2 and their percentiles in the histogram.

FIG. 33A shows an example scatter plot of normalized fitted W2 and normalized target W2 for 1435 discrete optimal translations.

FIG. 33B shows an example scatter plot of normalized fitted W2 and normalized target W2 for 1435 discrete optimal translations by residual distance tertile.

FIG. 33C shows an example histogram of KNN indices of target perturbation to fitted perturbation for 1435 discrete optimal translations.

FIG. 33D shows an example histogram of percentiles of W2 distances between the latent spaces of the real cells treated by fitted perturbation and target perturbation in the distribution of the W2 distances between the latent spaces of the real cells treated by target perturbation and other perturbations across the 1435 discrete optimal translations.

FIG. 33E shows an example histogram of the W2 distances between the target latent space of a drug treatment and the translated latent space from the cells treated by a starting drug treatment to each of the 205 observed drug treatments, along with fitted W2, target W2 and their percentiles in the histogram.

FIG. 34 shows example UMAP plots of latent values of K562 cells treated by the starting perturbation S1122 with dose 100, target perturbation S2692 with dose 100, fitted perturbation S2736 with dose 100, as well as translated latent values from K562 cells treated by S1122 with dose 100 to S2736 and S2692.

FIG. 35 shows example UMAP plots of latent values of cells treated by the starting perturbation G1, target perturbation G2, fitted perturbation G3, as well as translated latent values from cells treated by G1 to G3 and G2.

FIG. 36A-36D show model interpretation of the chemical perturbation G1 for latent clustering of the LINCS-Drug data. Specifically, FIG. 36A shows an example UMAP plot of latent values. FIG. 36B shows an example UMAP plot of latent values by cluster label assigned by k-means clustering with k=20. FIG. 36C shows example UMAP plots of latent clusters 4, 11, 16 and 20. FIG. 36D shows example molecular structures of G1 colored by atomic attributions to the formations of latent clusters 4, 11, 16 and 20.

FIG. 37 illustrates an example overview of interpreting perturbations for latent clustering.

FIG. 38A-38D show example model interpretation of the genetic perturbation ‘ERG’ for latent clustering of the LINCS-Gene for clusters 9 and 17. Specifically, FIG. 38A shows an example UMAP plot of latent values. FIG. 38B shows an example UMAP plot of latent values by cluster label assigned by k-means clustering with k=20. FIG. 38C shows example UMAP plots of latent clusters 9 and 17. FIG. 38D shows example bar plots of the 10 highest attributions of gene ontology (GO) annotations colored by being in ERG or not, with percentages in baseline perturbations for clusters 9 and 17.

FIG. 39A shows example plots of GO terms from the attributions of the genetic perturbation with target gene ERG for forming latent clusters 9 and 17, showing biological process, and molecular function.

FIG. 39B shows example plots of GO terms from the attributions of the genetic perturbation with target gene ERG for forming latent clusters 9 and 17, showing cellular component.

FIG. 40A-40D show example model interpretation of the genetic perturbation ERG for latent clustering of LINCS-Gene for clusters 1 and 5. Specifically, FIG. 40A shows example UMAP plots of latent clusters 1 and 5. FIG. 40B shows example bar plots of the 10 highest attributions of GO annotations colored by being in ERG or not, with percentages in baseline perturbations for clusters 1 and 5. FIG. 40C shows example plots of GO terms from the attributions of the genetic perturbation with target gene ERG for generating latent clusters 1 and 5, showing biological process, and molecular function. FIG. 40D shows example plots of GO terms from the attributions of the genetic perturbation with target gene ERG for generating latent clusters 1 and 5, showing cellular component.

FIG. 41 shows an example overview of interpreting perturbations for optimal translations.

FIGS. 42A-42C3 show example model interpretation for three discrete optimal translations of the sci-Plex. Specifically, FIG. 42A shows an example scatter plot of normalized fitted W2 and normalized target W2 for 18,960 discrete optimal translations and three selected scenarios. FIG. 42B1 shows example molecular structures of fitted and target perturbations colored by atomic attributions to translate the starting latent space to the target latent space for two of the scenarios (e.g., scenarios S1 and S2). FIG. 42B2 shows example molecular structures of fitted and target perturbations colored by atomic attributions to translate the starting latent space to the target latent space for the third scenario (e.g., scenario S3). FIG. 42C1 shows example bar plots of attributions of perturbation representations of fitted and target perturbations to translate the starting latent space to the target latent space for the first scenario (e.g., scenario S1). FIG. 42C2 shows example bar plots of attributions of perturbation representations of fitted and target perturbations to translate the starting latent space to the target latent space for the second scenario (e.g., scenario S2). FIG. 42C3 shows example bar plots of attributions of perturbation representations of fitted and target perturbations to translate the starting latent space to the target latent space for the third scenario (e.g., scenario S3).

FIGS. 43A-43C show example model interpretation for a discrete optimal translation of the LINCS-Drug. Specifically, FIG. 43A shows an example scatter plot of normalized fitted W2 and normalized target W2 for 1435 discrete optimal translations and the selected scenario. FIG. 43B shows example bar plots of attributions of perturbation representations of fitted and target perturbations to translate the starting latent space to the target latent space for the scenario. FIG. 43C shows example molecular structures of fitted and target perturbations colored by atomic attributions to translate the starting latent space to the target latent space for the scenario.

FIG. 44 shows an example overview of interpreting perturbations for shifting cell state distributions.

FIGS. 45A-45C2 show example model interpretation of pairs of genetic perturbations for shifting cell state distributions. Specifically, FIG. 45A shows example UMAP plots of latent values of cells treated by three pairs of genetic perturbations. FIG. 45B1 shows an example bar plot of the 10 highest attributions of GO annotations colored by being in the input perturbation or not, for a first pair of perturbations. Figure shows an example bar plot of the 10 highest attributions of GO annotations colored by being in the input perturbation or not, for a second pair of perturbations. FIG. 45B3 shows an example bar plot of the 10 highest attributions of GO annotations colored by being in the input perturbation or not, for a third pair of perturbations. Figure shows example plots of GO terms from the attributions of a genetic perturbation for shifting the cell state of a baseline perturbation to its cell state for the three pairs of perturbations, showing biological process, and molecular function. FIG. 45C2 shows example plots of GO terms from the attributions of a genetic perturbation for shifting the cell state of a baseline perturbation to its cell state for the three pairs of perturbations, showing cellular component.

FIG. 46 illustrates an example method for identifying a perturbation.

FIG. 47 illustrates an example method for identifying a drug treatment or protein sequence.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

The present embodiments relate to, among other things, identifying a perturbation that will cause a starting cell state to transition to a target cell state.

In some examples, it may be desirable to identify a perturbation that transitions a cell in a diseased state (e.g., a starting cell state) to a healthy cell state (e.g., a target cell state). To this end, some embodiments input a starting cell state and a target cell state into a trained machine learning algorithm, which identifies a perturbation that causes the starting cell state to transition to the target cell state.

In this regard, some embodiments consider two types of perturbations: chemical perturbations, and genetic perturbations. Chemical perturbations are usually delivered as a drug treatment on cells.

Genetic perturbations, on the other hand, are performed directly on genes to impact their functions. One example of this is an application of clustered regularly interspaced short palindromic repeats (CRISPR), which is a family of proteins that contribute to the immune system of prokaryotic organisms. CRISPR has been adapted to perform gene-editing with Cas9, a widely used protein in bacteria that can easily find and bind to most of the desired sequences with a piece of guide RNA (Jinek, M., K. Chylinski, I. Fonfara, M. Hauer, J. A. Doudna, and E. Charpentier (2012), A programmable dual-rna-guided dna endonuclease in adaptive bacterial immunity, science, 337(6096), 816-821). The CRISPR/Cas9 system has been shown to perform RNA-guided site-specific DNA cleavages (Cong, L., et al. (2013), Multiplex genome engineering using crispr/cas systems, Science, 339(6121), 819-823). The CRISPR/Cas9 method delivers Cas9, guide RNA, and an associated complex to cells. The specified guide RNA molecule can locate a particular segment of host DNA and bind with the protospacer adjacent motif (PAM) sequence to induce double-stranded breaks of a DNA sequence. The Cas9 enzyme then precisely knocks out target genomic loci. The cleaved DNA sequences are repaired to form new sequences. The CRISPR/Cas9 method also facilitates simultaneous cleavages of multiple target loci, improving the efficiency and flexibility of gene editing activity (Bialk, P., N. Rivera-Torres, B. Strouse, and E. B. Kmiec (2015), Regulation of gene editing activity directed by single-stranded oligonucleotides and crispr/cas9 systems, PloS one, 10(6), e0129,308).

Furthermore, chemical and genetic perturbations may be used in screen experiments. In this regard, a screen experiment usually involves multiple complex chemical or genetic perturbations that potentially change cells' phenotypic characteristics (Devlin, J. P. (1997), High throughput screening: the discovery of bioactive substances, CRC Press), High throughput screening: the discovery of bioactive substances, CRC Press). Chemical perturbations are widely used to impact cellular activities, and a chemical perturbation is usually delivered as a drug treatment on cells.

The high-throughput screen (HTS) technologies originated in natural product screening and were then utilized to identify molecules that modulated therapeutic targets (Pereira, D., and J. Williams (2007), Origin and evolution of high throughput screening, British journal of pharmacology, 152(1), 53-61). HTS experienced steady improvements to incorporate absorption, distribution, metabolism and excretion (ADME) targets (Banker, M. J., T. H. Clark, and J. A. Williams (2003), Development and validation of a 96-well equilibrium dialysis apparatus for measuring plasma protein binding, Journal of pharmaceutical sciences, 92(5), 967-974.). Advances of scalability and efficiency of HTS have enabled many new methods for pharmaceutical drug discovery (Janzen, W. P. (2001), High throughput screening: methods and protocols, 190, Springer Science & Business Media; Minor, L. K. (2006), Handbook of assay development in drug discovery, CRC Press).

HTS technologies have been employed to identify cellular elements with proliferation properties (Yu, C., et al. (2016), High-throughput identification of genotype-specific cancer vulnerabilities in mixtures of barcoded tumor cell lines, Nature biotechnology, 34(4), 419-423). Researchers have also implemented HTS experiments in imaging responses with high-content morphology responses (Perlman, Z. E., M. D. Slack, Y. Feng, T. J. Mitchison, L. F. Wu, and S. J. Altschuler (2004), Multidimensional drug profiling by automated microscopy, Science, 306(5699), 1194-1198). Additionally, HTS identifies optimal cell lines with highly specific readouts (Kang, J., C.-H. Hsu, Q. Wu, S. Liu, A. D. Coster, B. A. Posner, S. J. Altschuler, and L. F. Wu (2016), Improving drug discovery with high-content phenotypic screens by systematic selection of reporter cell lines, Nature biotechnology, 34(1), 70-77).

### Example System

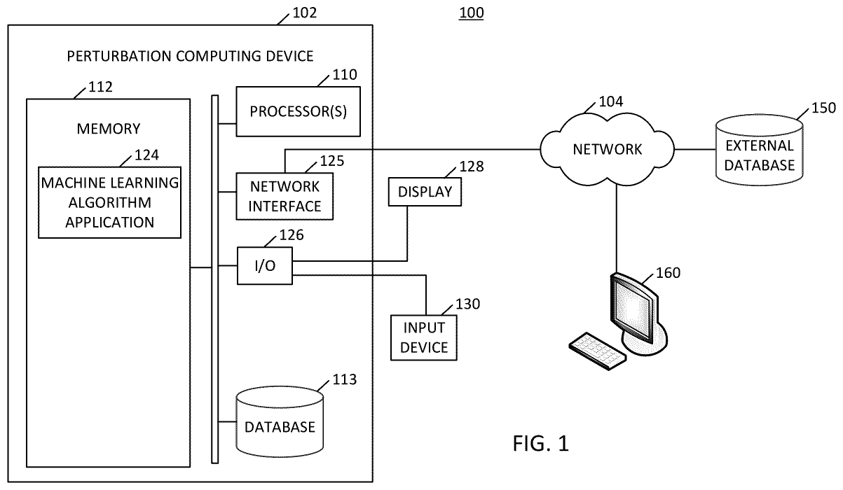

FIG. 1 illustrates an example architecture 100 that may be used for identifying a perturbation that will cause a starting cell state to transition to a target cell state.

The architecture 100 may include a perturbation computing device 102 configured to communicate, e.g., via a network 104 (which may be a wired or wireless network, such as the internet), with an external database 150.

The perturbation computing device 102 may include one or more processor(s) 110, which may comprise Central Processing Units (CPUs), and/or on one or more or Graphical Processing Units (GPUs), including clusters of CPUs and/or GPUs, and/or one or more tensor processing unites (TPU). Features and functions described for the perturbation computing device 102 may be stored on and implemented from one or more memory 112 (e.g., non-transitory computer-readable media) of the perturbation computing device 102. The memory 112 may include, for example, a machine learning algorithm application 124 (e.g., which may train and/or use machine learning algorithm(s) to identify a perturbation). More generally, the memory 112 may use the components it includes to implement the techniques described herein. The memory 112 and the one or more processors 110 may store cell states, perturbations, or any other data in one or more databases 113.

The perturbation computing device 102 includes a network interface 125 communicatively coupled to the network 104, for communicating to and/or from a portable personal computer 160, or any other device, such as a smart phone, etc. not shown in FIG. 1. The perturbation computing device 102 further includes an I/O interface 126 connected to devices, such as digital displays 128, user input devices 130, etc. In some examples, as described herein, the perturbation computing device 102 identifies a perturbation that will cause a starting cell state to transition to a target cell state. In the illustrated example, the perturbation computing device 102 is implemented as a device. However, the functions of the perturbation computing device 102 may be implemented across other computing devices, etc. connected to one another through a communication link or through the network 104. In other examples, the functions of the perturbation computing device 102 may be cloud based, such as, for example one or more connected cloud TPU(s) customized to perform machine learning processes. Furthermore, although the example of FIG. 1 shows only one perturbation computing device 102, any number of perturbation computing devices 102 may be used (e.g., the functions of the perturbation computing device 102 may be distributed across any number of devices).

The network 104 may be a public network such as the Internet, private network such as research institution's or corporation's private network, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), or other types of protocols. Moreover, the network 104 can include a number of devices that facilitate network communications and/or form a hardware basis for the networks, such as switches, routers, gateways, access points (such as a wireless access point as shown), firewalls, base stations, repeaters, backbone devices, etc.

The memory 112 may include executable computer-readable code stored thereon for programming a computer (e.g., comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the perturbation computing device 102 may represent a CPU-type processing unit, a GPU-type processing unit, a TPU-type processing unit, a field-programmable gate array (FPGA), another class of digital signal processor (DSP), or other hardware logic components that can be driven by a CPU.

The external database 150 may hold any data used by or created by the perturbation computing device 102. For example, the external database 150 may store perturbations identified by the perturbation computing device 102, starting cell states, target cell states, lists of perturbations, patent data, etc. Other examples of data stored by the external database 150 include data from experiments, such as high-throughput chemical screen experiments, as described elsewhere herein.

### Measuring Single-Cell Responses to Perturbations

To train the machine learning algorithm (e.g., via the machine leaning algorithm application 124), data from experiments, such as from a high-throughput chemical screen experiment, may be used.

In a high-throughput chemical screen experiment, many cells receive drug treatments, and then their single-cell transcriptional responses are measured using single-cell technologies. A high-throughput chemical screen experiment, such as sci-Plex, can collect scRNA-seq responses to hundreds of drugs on multiple cell types (Srivatsan, S. R., et al. (2020), Massively multiplex chemical transcriptomics at single-cell resolution, Science, 367(6473), 45-51). The single-cell responses to chemical perturbations can be represented via cell by gene matrices, with each entry representing the gene's transcription count in the cell. The identity and dose of the drug each cell receives are known.

Single-cell technologies also enable measuring the effects of using CRISPR gene editing methods to knock out, activate or knock down target genes. These CRISPR perturbations modify the activities of other genes through a complex network of genetic interactions in which genes regulate each other's expression levels (Statello, L., C.-J. Guo, L.-L. Chen, and M. Huarte (2021), Gene regulation by long non-coding rnas and its biological functions, Nature Reviews Molecular Cell Biology, 22(2), 96-118). As a result, a genetic perturbation exerts changes in the overall gene expression profile and molecular state of a cell. The Perturb-seq technologies combine scRNA-seq and CRISPR/Cas9 (Dixit, A., et al. (2016), Perturb-seq: dissecting molecular circuits with scalable single-cell rna profiling of pooled genetic screens, cell, 167(7), 1853-1866) or combine scRNA-seq and CRISPRi (Adamson, B., et al. (2016), A multiplexed single-cell crispr screening platform enables systematic dissection of the unfolded protein response, Cell, 167(7), 1867-1882) to measure responses to genetic perturbations. As with sci-Plex, scRNA-seq responses to genetic perturbations are usually cell by gene matrices. The identity of the perturbation applied to each cell is known. Apart from Perturb-seq, other techniques such as CRISP-seq and CROP-seq also combine CRISPR perturbations with scRNA-seq, but have slightly different experimental and technical approaches (Jaitin, D. A., et al. (2016), Dissecting immune circuits by linking crispr-pooled screens with single-cell rna-seq, Cell, 167(7), 1883-1896; Datlinger, P., et al. (2017), Pooled crispr screening with single-cell transcriptome readout, Nature methods, 14(3), 297-301).

The effects of CRISPR perturbations can also be measured with single-cell epigenomic profiling (Rubin, A. J., et al. (2019), Coupled single-cell crispr screening and epigenomic profiling reveals causal gene regulatory networks, Cell, 176(1-2), 361-376) and single-cell multimodal profiling (Mimitou, E. P., et al. (2019), Multiplexed detection of proteins, transcriptomes, clono-types and crispr perturbations in single cells, Nature methods, 16(5), 409-412; Frangieh, C. J., et al. (2021), Multimodal pooled perturb-cite-seq screens in patient models define mechanisms of cancer immune evasion, Nature genetics, 53(3), 332-341; Papalexi, E., et al. (2021), Characterizing the molecular regulation of inhibitory immune checkpoints with multimodal single-cell screens, Nature genetics, 53(3), 322-331). CaRPool-seq combines CRISPR perturbations with multimodal single-cell profiling (Wessels, H.-H., et al. (2022), Efficient combinatorial targeting of rna transcripts in single cells with cas13 rna perturb-seq, bioRxiv). In addition, single-cell imaging data with perturbations can reveal substantial information for mining and identifying cellular mechanisms (Danuser, G. (2011), Computer vision in cell biology, Cell, 147(5), 973-978). CRISPR methods can also be paired with morphological profiling at scale in image-based screens (Feldman, D., A. Singh, J. L. Schmid-Burgk, R. J. Carlson, A. Mezger, A. J. Garrity, F. Zhang, and P. C. Blainey (2019), Optical pooled screens in human cells, Cell, 179(3), 787-799). High-throughput cell painting experiments can also incorporate chemical or genetic perturbations to study cell responses and identify potential therapeutics (Perlman, Z. E., M. D. Slack, Y. Feng, T. J. Mitchison, L. F. Wu, and S. J. Altschuler (2004), Multidimensional drug profiling by automated microscopy, Science, 306(5699), 1194-1198).

### Predicting Single-Cell Responses to Perturbations

The set of perturbations is usually limited in both chemical and genetic screen experiments. Although the delivery of a drug treatment is experimentally easier, a multiplex chemical experiment usually involves several drug treatments and each drug treats a large number of cells with different experimental conditions (Gehring, J., J. Hwee Park, S. Chen, M. Thomson, and L. Pachter (2020), Highly multiplexed single-cell rna-seq by dna oligonucleotide tagging of cellular proteins, Nature Biotechnology, 38(1), The CRIPSR genetic perturbations have much more byzantine procedures where the perturbations are usually delivered with deliberately planned biomedical processes. The single-cell CRISPR screen experiments generally have been limited to studying a few hundred genetic perturbations (Replogle, J. M., et al. (2021), Mapping information-rich genotype-phenotype landscapes with genome-scale perturb-seq, bioRxiv), while the genome contains tens of thousands genes, leaving behind massive combinations of genes. Therefore, a model, such as described herein, that can predict single-cell responses to various unobserved perturbations is tremendously valuable.

Predicting single-cell responses to perturbations, however, has some unique challenges. First, cells have heterogeneous responses to a perturbation (Altschuler, S. J., and L. F. Wu (2010), Cellular heterogeneity: do differences make a difference?, Cell, 141(4), 559-563; Snijder, B., and L. Pelkmans (2011), Origins of regulated cell-to-cell variability, Nature reviews Molecular cell biology, 12(2), 119-125) and show varying individual perturbation responses. A more specific focus of perturbation responses might involve the individual treatment effect (ITE) (Shalit, U., F. D. Johansson, and D. Sontag (2017), Estimating individual treatment effect: generalization bounds and algorithms, in International Conference on Machine Learning, pp. 3076-3085, PMLR) in causal inference to predict single-cell perturbation responses of an individual cell or a cell subpopulation. Second, measuring single-cell responses usually destroys the cells, and each cell only provides a static snapshot of its profile after a specified perturbation. Third, the assignment of perturbations might be correlated with some cell state covariates characterizing the cell subpopulation under each perturbation, which further confounds attempts to model the perturbation responses. A model accurately predicting individual single-cell perturbation responses needs to debias the confounding effects from between-perturbation cellular characteristics.

### Deep Generative Models

Machine learning models have been developed for single-cell data. Two main branches of machine learning models are discriminative models and generative models. A discriminative model, like a classification tree, predicts target variables from observable variables. On the other hand, a generative model, such as a Gaussian mixture model (GMM), learns to generate the observed variables from latent variables. Generative models give a way of understanding the real-world data, and their latent variables serve as representations learned from the observable variables. However, most of the machine-learning-based generative models have limited performance to generate complex high-dimensional data such as those in single-cell transcriptome or imaging.

The deep learning era brought much more powerful generative models that are constructed with deep neural networks from latent variables to data samples, and are stochastically optimized. Some of the early deep generative models such as restricted Boltzmann machines (Hinton, G. E. (2002), Training products of experts by minimizing contrastive divergence, Neural computation, 14(8), 1771-1800) and sigmoid belief networks (Neal, R. M. (1992), Connectionist learning of belief networks, Artificial intelligence, 56(1), 71-113) were very efficient in data modeling (Welling, M., M. Rosen-Zvi, and G. E. Hinton (2004), Exponential family harmoniums with an application to information retrieval, Advances in neural information processing systems, 17) and their extensions have been applied to different types of data (Hinton, G. E., S. Osindero, and Y.-W. Teh (2006), A fast learning algorithm for deep belief nets, Neural computation, 18(7), 1527-1554; Hinton, G. E., and R. R. Salakhutdinov (2009), Replicated softmax: an undirected topic model, Advances in neural information processing systems, 22; Salakhutdinov, R., and H. Larochelle (2010), Efficient learning of deep boltzmann machines, in Proceedings of the thirteenth international conference on artificial intelligence and statistics, pp. 693-700, JMLR Workshop and Conference Proceedings; Mnih, A., and K. Gregor (2014), Neural variational inference and learning in belief networks, in International Conference on Machine Learning, pp. 1791-1799, PMLR). Three commonly-used classes of deep generative models are variational autoencoders (VAEs) (Kingma, D. P., and M. Welling (2013), Auto-encoding variational bayes, arXiv preprint arXiv:1312.6114), generative adversarial networks (GANs) (Goodfellow, I., J. Pouget-Abadie, M. Mirza, B. Xu, D. Warde-Farley, S. Ozair, A. Courville, and Y. Bengio (2014), Generative adversarial nets, in Advances in neural information processing systems, pp. 2672-2680) and auto-regressive models such as PixeICNN and PixeIRNN (Oord, A. v. d., N. Kalchbrenner, L. Espeholt, O. Vinyals, A. Graves, et al. (2016a), Conditional image generation with pixelcnn decoders, Advances in neural information processing systems, 29; Oord, A. v. d., N. Kalchbrenner, and K. Kavukcuoglu (2016b), Pixel recurrent neural networks, in International conference on machine learning, pp. 1747-1756, PMLR; Oord, A. v. d., S. Dieleman, H. Zen, K. Simonyan, O. Vinyals, A. Graves, N. Kalch-brenner, A. Senior, and K. Kavukcuoglu (2016c), Wavenet: A generative model for raw audio, arXiv preprint arXiv:1609.03499).

These state-of-the-art deep generative models have achieved tremendous success in the domains of images and natural language. Frameworks such as InfoGAN learn interpretable latent representations from images that manipulate semantically meaningful image generation (Chen, X., Y. Duan, R. Houthooft, J. Schulman, I. Sutskever, and P. Abbeel (2016), Infogan: Interpretable representation learning by information maximizing generative adversarial nets, in Advances in neural information processing systems, pp. 2172-2180). Many derivatives of GANs such as WGAN (Arjovsky, M., S. Chintala, and L. Bottou (2017), Wasserstein gan, arXiv preprint arXiv:1701.07875), Progressive GAN (Karras, T., T. Aila, S. Laine, and J. Lehtinen (2017), Progressive growing of gans for improved quality, stability, and variation, arXiv preprint arXiv:1710.10196) and LOGAN (Wu, Y., J. Donahue, D. Balduzzi, K. Simonyan, and T. Lillicrap (2019), Logan: Latent optimisation for generative adversarial networks, arXiv preprint arXiv:1912.00953) improve the training stability and generation quality for high-resolution images. Conditional generation enables text-to-image synthesis (Reed, S., Z. Akata, X. Yan, L. Logeswaran, B. Schiele, and H. Lee (2016), Generative adversarial text to image synthesis, arXiv preprint arXiv:1605.05396) or image-to-image translation (Isola, P., J.-Y. Zhu, T. Zhou, and A. A. Efros (2017), Image-to-image translation with conditional adversarial networks, in Proceedings of the IEEE conference on computer vision and pattern recognition, pp. 1125-1134; Zhu, J.-Y., T. Park, P. Isola, and A. A. Efros (2017), Unpaired image-to-image translation using cycle-consistent adversarial networks, in Proceedings of the IEEE international conference on computer vision, pp. 2223-2232). VAEs coupled with normalizing flows improve variational inference for images (Rezende, D., and S. Mohamed (2015), Variational inference with normalizing flows, in International conference on machine learning, pp. 1530-1538, PMLR; Kingma, D. P., T. Salimans, R. Jozefowicz, X. Chen, I. Sutskever, and M. Welling (2016), Improved variational inference with inverse autoregressive flow, Advances in neural information processing systems, 29). Conditional VAEs can learn text attributes to perform attribute specific data generation or interpolation (Yan, X., J. Yang, K. Sohn, and H. Lee (2016), Attribute2image: Conditional image generation from visual attributes, in European conference on computer vision, pp. 776-791, Springer). VAEs can also generate coherent novel sentences from interpretable latent representations (Bowman, S. R., L. Vilnis, O. Vinyals, A. M. Dai, R. Jozefowicz, and S. Bengio (2015), Generating sentences from a continuous space, arXiv preprint arXiv:1511.06349). In addition, the normalizing flows of autoregressive models delineate the complex high-dimensional densities of images (Dinh, L., D. Krueger, and Y. Bengio (2014), Nice: Non-linear independent components estimation, arXiv preprint arXiv:1410.8516; Dinh, L., J. Sohl-Dickstein, and S. Bengio (2016), Density estimation using real nvp, arXiv preprint arXiv:1605.08803; Grover, A., M. Dhar, and S. Ermon (2018), Flow-gan: Combining maximum likelihood and adversarial learning in generative models, in Proceedings of the AAAI Conference on Artificial Intelligence, vol. 32). More recently, models such as transformers achieve excellent performance in both natural language processing and computer vision (Vaswani, A., N. Shazeer, N. Parmar, J. Uszkoreit, L. Jones, A. N. Gomez, L. Kaiser, and I. Polosukhin (2017), Attention is all you need, Advances in neural information processing systems, 30; Brown, T., et al. (2020), Language models are few-shot learners, Advances in neural information processing systems, 33, 1877-1901). Single-cell data have also utilized deep generative models to generate realistic samples and learn representations. The scVl framework (Lopez, R., J. Regier, M. B. Cole, M. I. Jordan, and N. Yosef (2018), Deep generative modeling for single-cell transcriptomics, Nature methods, 15(12), 1053-1058) combines probabilistic modeling of scRNA-seq data and variational inference to learn low-dimensional latent representations and generate realistic single-cell responses. Other methods have also facilitated the prediction of single-cell responses to perturbations (Lotfollahi, M., F. A. Wolf, and F. J. Theis (2019), scgen predicts single-cell perturbation responses, Nature methods. 16(8), 715-721). The current deep generative models for single-cell perturbation experiments, however, are usually unable to deal with the heterogeneity of perturbation responses, identification of pre-perturbation cell state distributions, or confounding bias in modeling perturbation responses. Additionally, most of existing methods only deal with limited numbers of perturbations, and can hardly extend to unseen perturbations. Therefore, new methods that effectively learn pre-perturbation cell state information, and accurately model single-cell perturbation responses for various perturbations, are needed.

### Overview

As described herein, deep generative models are developed for modeling single-cell perturbation data. One aim is to accurately capture the cell state of individual cells from their single-cell perturbation responses. Some embodiments then precisely predict single-cell responses to various chemical or genetic perturbations.

More specifically, some embodiments propose a novel deep generative model to predict single-cell responses to unseen drug treatments. The deep generative model precisely captures drug information and cell state, as well as their relationships. Also proposed is a machine-learning method and predictions of scRNA-seq responses to drug treatments observed in the training data or from an unseen set using the proposed methods. Also shown is that adjusting for cell state covariates can improve the prediction performance. Also developed is a fine-tuning technique for the framework to improve its prediction performance.

Furthermore, some embodiments extend the proposed methods for drug treatments to predict single-cell responses to genetic perturbations. In this regard, some embodiments consider two kinds of genetic perturbations: gene knockout/knockdown and sequence mutation. Some embodiments predict single-cell perturbation responses for several large high-throughput screen datasets with genetic perturbations. Some embodiments also fine-tune the framework and show improved performance of the methods.

Some implementations develop methods to design optimal perturbations and interpret the trained model. This considers the scenarios of finding alternative perturbations to translate cells to approximate a target cell state. Some embodiments extend the disclosed deep generative models for chemical and genetic perturbations to predict the counterfactual responses of cells. In addition, some embodiments also employ model interpretability methods to identify important atoms or gene ontology terms within chemical and genetic perturbations.

### Predicting Single-Cell Responses to Drug Perturbations

### INTRODUCTION

Recent experimental developments have enabled high-throughput single-cell molecular measurement of response to drug treatment. A high-throughput chemical screen experiment usually involves a large number of cells and multiple treatments, where each cell receives a kind of drug treatment and is impacted in a distinct manner (Gehring, J., J. Hwee Park, S. Chen, M. Thomson, and L. Pachter (2020), Highly multiplexed single-cell rna-seq by dna oligonucleotide tagging of cellular proteins, Nature Biotechnology, 38(1), 35-38; Srivatsan, S. R., et al. (2020), Massively multiplex chemical transcriptomics at single-cell resolution, Science, 367(6473), 45-51).

Understanding how drugs influence cellular responses helps discover treatments with desired effects, potentially benefiting a myriad of therapeutic applications. Various methods have been developed to predict cellular responses under drug treatments, including mechanistic models on protein level changes and phenotype changes (Yuan, B., C. Shen, A. Luna, A. Korkut, D. S. Marks, J. Ingraham, and C. Sander (2021), Cellbox: interpretable machine learning for perturbation biology with application to the design of cancer combination therapy, Cell systems, 12(2), 128-140), as well as deep learning models on disease outcomes (Cheng, F., I. A. Kovács, and A.-L. Barabási (2019), Network-based prediction of drug combinations, Nature communications, 10(1), 1-11; Kuenzi, B. M., J. Park, S. H. Fong, K. S. Sanchez, J. Lee, J. F. Kreisberg, J. Ma, and T. Ideker (2020), Predicting drug response and synergy using a deep learning model of human cancer cells, Cancer cell, 38(5), 672-684).

Some embodiments predict the conditional distribution of cell states given different drug treatments. In a particular, tissue under homeostatic conditions, there is a wild-type distribution of cellular gene expression states p(X). However, treating cells with a drug G changes their cell state distribution. One goal is to shape these treatment-specific data distributions p(X|G) by developing deep generative models to sample from the cell state distribution for any drug treatment.

Several deep generative models have generated realistic single-cell data from drug treatments. One work (Lotfollahi, M., M. Naghipourfar, F. J. Theis, and F. A. Wolf (2020), Conditional out-of-distribution generation for unpaired data using transfer vae, Bioinformatics, 36(Supplement 2), i610-i617) proposed a conditional variational autoencoder (VAE) framework with representations under two treatment conditions balanced using a similarity score of their counterfactual inference developed by another work (Johansson, F., U. Shalit, and D. Sontag (2016), Learning representations for counter-factual inference, in International conference on machine learning, pp. 3020-3029, PMLR). In contrast, scGen predicts single-cell data through latent space vector arithmetic in an unsupervised learning fashion (Lotfollahi, M., F. A. Wolf, and F. J. Theis (2019), scgen predicts single-cell perturbation responses. Nature methods, 16(8), 715-721). Another method (Rampášek L., D. Hidru, P. Smirnov, B. Haibe-Kains, and A. Goldenberg (2019), Dr. vae: improving drug response prediction via modeling of drug perturbation effects, Bioinformatics, 35(19), 3743-3751) also explored the dependency of the latent space on treatments. These methods, however, can only apply to drug treatment experiments with two treatment conditions, and they are unable to make predictions for unseen drug treatments. In response, a work (Lotfollahi, M., A. K. Susmelj, C. De Donno, Y. Ji, I. L. Ibarra, F. A. Wolf, N. Yakubova, F. J. Theis, and D. Lopez-Paz (2021), Compositional perturbation autoencoder for single-cell response modeling, bioRxiv) proposed compositional perturbation autoencoder (CPA) to extract a basal state in the latent space of VAE, and to further predict latent values under drug treatments using a linear model. However, CPA assumes that it is possible to learn the effect of a perturbation independent from the cell state, which is probably invalid in many cases. For example, if a perturbation G selectively kills cells in state A, the model will incorrectly generate A cells under perturbation G. In contrast, systems and methods described herein can model general relationships between drug treatment and cell state, whether dependent or independent.

One proposal disclosed herein is PerturbNet, a novel and flexible framework that predicts single-cell responses to different perturbations. The PerturbNet model connects drug treatment information and latent space through normalizing flows (Papamakarios, G., E. Nalisnick, D. J. Rezende, S. Mohamed, and B. Lakshmi-narayanan (2021), Normalizing flows for probabilistic modeling and inference, Journal of Machine Learning Research, 22(57), 1-64), enabling the translation between drug treatment domain and single-cell domain (Baltrušaitis T., C. Ahuja, and L.-P. Morency (2018), Multimodal machine learning: A survey and taxonomy, IEEE transactions on pattern analysis and machine intelligence, 41 (2), 423-443). The PerturbNet framework is generally applicable to any type of data with drug treatments, such as those of single-cell RNA-seq (scRNA-seq) data. Furthermore, PerturbNet can make predictions for both observed and unseen drug treatments.

### Methods

Drug Treatment Encoder and Chemical Variational Autoencoder

The known one-hot encoding approach can transform drug treatment labels to a vector of 1's and 0's, but requires pre-specifying of the total number of possible drug treatments and cannot encode new treatments after the specification. Therefore, some embodiments consider flexible representations Y for drug treatments to predict drug treatment effects on single-cell data for unseen perturbations.

A drug treatment contains abundant information more than just a label, such as ‘S1096’. Its pharmacological properties are usually determined by its chemical structure. Some embodiments thus aim to encode drugs' chemical structures to dense representations. Some embodiments consider drug treatments' simplified molecular-input line-entry system (SMILES) strings, which distinctively represent chemical structures and treatment information. Although SMILES strings can be encoded to numerical representations through molecular Morgan fingerprints (Rogers, D., and M. Hahn (2010), Extended-connectivity fingerprints, Journal of chemical information and modeling, 50(5), 742-754) or through language models (Xu, Z., S. Wang, F. Zhu, and J. Huang (2017), Seq2seq fingerprint: An unsupervised deep molecular embedding for drug discovery, in Proceedings of the 8th ACM international conference on bioinformatics, computational biology, and health informatics, pp. 285-294; Chithrananda, S., G. Grand, and B. Ramsundar (2020), Chemberta: Large-scale self-supervised pretraining for molecular property prediction, arXiv preprint arXiv:2010.09885), the representations from these methods are deterministic, meaning that the representations remain the same in replicated encoding implementations. Given that a chemical screen experiment usually contains a limited number of distinct drug treatments, the use of stochastic representations of the drug treatments prevents possible model overfitting.

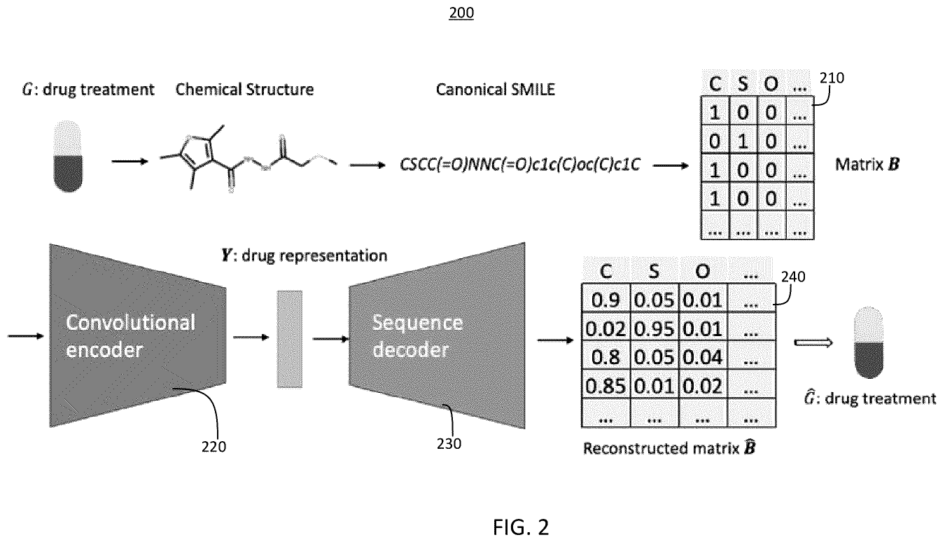

To improve the learning capacity, especially for representations of unseen treatments, some embodiments consider using a chemical variational autoencoder, such as the example chemical variational autoencoder 200 of FIG. 2, to generate the stochastic sampled representation Y of each drug's SMILES string (Kusner, M. J., B. Paige, and J. M. Hernandez-Lobato (2017), Grammar variational autoencoder, in International Conference on Machine Learning, pp. 1945-1954, PMLR; Zhu, J., et al. (2021), Prediction of drug efficacy from transcriptional profiles with deep learning, Nature Biotechnology, pp. 1-9). In essence, the chemical variational autoencoder 200 first transforms and standardizes SMILES strings to their canonical forms and tokenizes each canonical SMILES to be encoded as a one-hot matrix 210. For a canonical SMILES string, the ith row of its one-hot matrix 210 corresponds to its ith place, and has the jth column being 1 and all other columns being 0's, if its ith place has the jth character in the collected chemical elements' library. The one-hot matrices of SMILES strings are then fitted into an encoder 220 which provides representations Y for SMILES strings of drug treatments g. The decoder 230 may then be used to produce the reconstructed matrix 240 to thereby produce the drug treatment Ĝ.

Baseline KNN and Random Models

From the perturbation representations, Y, of drug treatments, some embodiments learn the relationship of several drug treatments in their latent space. Some embodiments assume that drug treatments with close latent values tend to also have similar single-cell responses. Thus, the distributions of perturbation responses p(X|G=g₁) and p(X|G=g₂) are similar if g₁ and g₂ have close representations of y₁ and y₂.

Some embodiments then propose a baseline model using the k-nearest neighbors (KNN) algorithm to predict single-cell data under drug treatments in Algorithm 1. From the chemical variational autoencoder, some embodiments obtain the representation Y for a set of treatments [symbol] , each of which has measured single-cell samples. Then for a drug treatment g∉ [symbol] with representation Y, one can find its k nearest neighbors {g₍₁₎, . . . , g₍ₖ₎} from G based on Y. Some embodiments then sample single-cell samples treated with the k nearest treatments in proportion to their exponentiated negative distances to the treatment of interest in the latent space of Y. The sampled single-cell data can be regarded as a baseline prediction for the single-cell data with the treatment of interest.

Algorithm 1: Baseline KNN Model:

Input: Drug treatment of interest g and its representation y. A set of drug
treatments [symbol] = {g₁, ... , gₘ} with their representations {y₁, ... , yₘ} as well as single-
cell sample sets {X₁, ... , Xₘ}.
1. Train KNN algorithm (k = 5) on {y₁, ... , yₘ}.
2. Obtain y's k neighbors {y₍₁₎, ... , y₍ₖ₎} and their pairwise distances (d₍₁₎, ... , d₍ₖ₎)^{T}
from the trained KNN algorithm.
3. Sample a number of cells X′ through stratified sampling with replacement from
{X₍₁₎, ... , X₍ₖ₎}. Each set X₍ᵢ₎ has a proportion of
exp{−d₍ᵢ₎}/Σⱼ₌₁^{k} exp{−d₍ⱼ₎}.
Result: predicted single cells X′ under perturbation g.

The key assumption of the baseline KNN model is that the perturbation representation is informative to infer single-cell data. To test the informativeness assumption on the perturbation representation, some embodiments propose a naive baseline random model in Algorithm 2 that randomly samples single-cell samples under treatments other than the target treatment. If the perturbation representation is uninformative to inferring cell state or cellular response, the random model is likely to have a similar performance to the KNN model.

Algorithm 2: Baseline Random Model

Input: Drug treatment of interest g. A set of single-cell samples {X_{-g}}
receiving drug treatments other than g.
1. Sample a number of cells X′ with replacement from X_{-g}.
Result: predicted single cells X′ under perturbation g.

Conditional Invertible Neural Networks

Some embodiments consider employing more complex normalizing flows of invertible neural networks to understand the relationship between perturbation representation and cellular responses. An affine coupling block (Dinh, L., J. Sohl-Dickstein, and S. Bengio (2016), Density estimation using real nvp, arXiv preprint arXiv:1605.08803) enables the input U=(U₁^{T}, U₂^{T}) to be transformed to output W=(W₁^{T},W₂^{T})^{T} with:

W₁=U₁⊙ exp{scale₁(U₂)}+trans₁(U₂)

W₂=U₂⊙ exp{scale₂(W₁)}+trans₂(W₁)

where scale₁(·), scale₂(·), trans₁(·), trans₂(·) are arbitrary scale and transformation neural networks, and ⊙ is the Hadamard product or element-wise product. The inverse of the coupling blocking can be represented by and

U₂={W₂−trans₂(W₁)}Ø exp{scale₂(W₁)}

and

U₁={W₂−trans₁(U₂)}Ø exp{scale₁(U₂)}

where Ø is the element-wise division. The affine coupling block allows bijective transformations between U and W with strictly upper or lower triangular Jacobian matrices. A conditional coupling block may be further adapted to concatenate a conditioning variable with inputs in scale and transformation networks. A conditional coupling block preserves the invertibility of the block and the simplicity of the Jacobian determinant.

A conditional invertible neural network (cINN) (Ardizzone, L., C. Lüth, J. Kruse, C. Rother, and U. Köthe (2019), Guided image generation with conditional invertible neural networks, arXiv preprint arXiv:1907.02392; Rombach, R., P. Esser, and B. Ommer (2020), Network-to-network translation with conditional invertible neural networks, arXiv preprint arXiv:2005.13580) is a type of conditional normalizing flow with conditional coupling blocks and actnorm layers (Kingma, D. P., and P. Dhariwal (2018), Glow: Generative flow with invertible 1×1 convolutions, Advances in neural information processing systems, 31), with both forward and inverse translations. Denote representations from two domains as Y∈ [symbol] _{Y} and Z∈ [symbol] _{Z}. A cINN modeling Z over Y gives forward translation

Z=f(V|Y)

and inverse translation

V=f⁻¹(Z|Y),

where V˜ [symbol] (0, I). cINN effectively models p(Z|Y), the probabilistic dependency of Z over Y with a residual variable V. As a cINN seeks to extract the shared information from Y and add residual information V to generate Z, the objective function to train a cINN is the Kullback-Leibler (KL) divergence between the residual's posterior q(V|Y) and its prior p(V). The objective function can further be derived to the following Equation (1):

[symbol]

_{p(Y)}[D_{KL}{q(V|Y)∥p(V)}]= [symbol] _{p(Z,Y)}[−log p{f⁻¹(V|Y)}−|detJ_{f}_{^{−1}}(Z|Y)|]−H(Z|Y)

where detJ_{f}_{^{−1}} is the determinant of the Jacobian matrix of f⁻¹ and H is a constant entropy. The optimal f that minimizes the objective function in Equation (1) gives q(V|Y)=p(V). In addition, the objective is an upper bound of the mutual information I(V,Y). Therefore, a well-trained cINN effectively achieves independence between V and Y. cINN has the same parameters for forward and inverse translations, reducing the number of model parameters while still preserving network details in both translation directions, and has been utilized to translate domain representations of images and texts (Rombach, R., P. Esser, and B. Ommer (2020), Network-to-network translation with conditional invertible neural networks, arXiv preprint arXiv:2005.13580).

PerturbNet

In some embodiments, from the chemical variational autoencoder, a drug treatment g is represented as a dense variable Y; some embodiments propose a baseline KNN model to make predictions for single-cell perturbation responses by sampling cells treated by similar perturbations according to their representations. To predict single-cell responses, a more powerful predictive model can be constructed with deep neural networks on perturbation representations. The deep learning predictive model can potentially give better predicting performance than the baseline KNN model, especially when it is trained with a large number of perturbations.

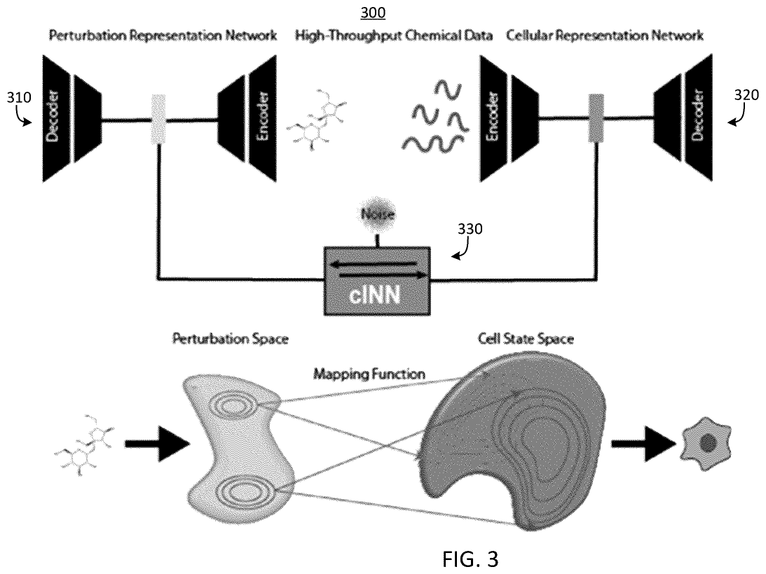

To this end, some embodiments propose a PerturbNet framework 300, an example of which is shown in FIG. 3. The PerturbNet framework has a VAE-based model for single-cell data and chemical variational autoencoder for drug treatments. The single-cell VAE model can be scVl (Lopez, R., J. Regier, M. B. Cole, M. I. Jordan, and N. Yosef (2018), Deep generative modeling for single-cell transcriptomics, Nature methods, 15(12), 1053-1058) for count data or regular VAE for normalized data. The pre-trained chemical variational autoencoder model encodes drug treatment G to perturbation representation Y (e.g., through first network 310). The pre-trained single-cell VAE model encodes single-cell sample X to cellular representation Z (e.g., through second network 320). Then, the perturbation representation Y and cellular representation Z are connected through a third network 330, which, in some embodiments, is a conditional invertible neural network (cINN). In some embodiments, in the third network 330, residual representation V predicts Z with Y as the conditioning variable. The residual representation is independent of perturbation in predicting cellular representation and is regarded as noise to the perturbation effects on cell state.

In addition, some embodiments also consider conditioning on known cell state covariates in modeling cell representation. Some embodiments concatenate perturbation representation and cell state covariates to serve as an overall condition of cINN and translate between residual or condition-invariant representation V and cellular representation. The extra conditioning on cell state covariates potentially debiases their confounding effects on modeling perturbation effects on cellular representation.

Chemical Variational Autoencoder Fine-Tuning

As both KNN and PerturbNet methods predict cell state based on perturbation representation, it might enhance the prediction performance for cell state from perturbation to use perturbation representation that learns cellular representation information.

Some embodiments propose an algorithm to fine-tune the chemical variational autoencoder, by adding the evidence lower bound (ELBO) loss of chemical variational autoencoder with an extra term for a certain cellular property quantity (Gómez-Bombarelli, R., et al. (2018), Automatic chemical design using a data-driven continuous representation of molecules, ACS central science, 4(2), 268-276). Some embodiments compute the Wasserstein-2 (W2) distance between cellular representations of each pair of perturbations and penalizing the quantity of the trace of Y's second moment weighted by the Laplacian matrix L of the adjacency matrix defined from pairwise distances (Cai, D., X. He, J. Han, and T. S. Huang (2010), Graph regularized nonnegative matrix factorization for data representation, IEEE transactions on pattern analysis and machine intelligence, 33(8), 1548-1560). Denote y and L as the perturbation representations and the Laplacian matrix of the perturbations. The penalizing quantity is defined as trace y^{T}Ly. By penalizing the proposed quantity, one may expect perturbations with similar cell states to have closer perturbation representations from chemical variational autoencoder. To implement the fine-tuning algorithm with cell state property, some embodiments alternate the chemical variational autoencoder training with a batch of chemical SMILES strings from a large chemical database with the ELBO loss and another batch of pairs of SMILES strings and cellular representations from a single-cell chemical screen dataset with the penalized ELBO loss. Some embodiments tune a hyperparameter λ on the extra term to adjust the chemical variational autoencoder fine-tuning performance. An example of the chemical variational autoencoder fine-tuning is summarized in Algorithm 3.

Algorithm 3: chemical variational autoencoder Fine-Tuning

```
Input: Set of drug treatments [symbol]    ₛ in ZINC or PubChem datasets. Single-cell samples
with perturbations {(x₁, g₁), ... , (xₙ, gₙ)}. The perturbations' cell-state Laplacian matrix
L. Two Adam optimizers (Kingma and Ba, 2014) Adam₁, Adam₂.
1. Initialize parameters (ϕ, θ).
2. While (ϕ, θ) has not converged:
  1). Sample a batch{(x₍ᵢ₎, g₍ᵢ₎)}ᵢ₌₁^{m} from the single-cell samples.
  2). Obtain representations y = (y₍₀₎, ... , y₍ₘ₎)^{T} for {g₍ᵢ₎}ᵢ₌₁^{m}
  3). Obtain the Laplacian matrix L_{g} for {g₍ᵢ₎}ᵢ₌₁^{m}.
  4). Compute gradient g_{ϕ,θ}^{λ} = ∇{−ELBO(ϕ, θ) + λtrace(y^{T} L_{g}y)}
  5). Update parameters using g_{ϕ,θ}^{λ} via Adam₁.
  6). Sample a batch {g₍ᵢ₎}ᵢ₌₁^{m}, from [symbol]    ₛ
  7). Compute gradient g_{ϕ,θ} = ∇_{ϕ,θ}{−ELBO(ϕ, θ)}
  8). Update parameters using g_{ϕ,θ}via Adam₂.
Result: fine-tuned chemical variational autoencoder with parameters (ϕ, θ).
```

Related Work

There has been limited previous work on predicting single-cell responses to multiple perturbations. A related work is compositional perturbation autoencoder (CPA, Lotfollahi, M., A. K. Susmelj, C. De Donno, Y. Ji, I. L. Ibarra, F. A. Wolf, N. Yakubova, F. J. Theis, and D. Lopez-Paz (2021), Compositional perturbation autoencoder for single-cell response modeling, bioRxiv), a VAE-based framework, which also models cellular representation under cellular perturbation. The CPA framework assumes that perturbation and known cell state covariates independently influence cellular representation in a linear model. In contrast, PerturbNet models cellular representation with normalizing flows, which flexibly incorporates perturbation, cell state covariates and also their complex interaction effects. In addition, the residual representation in Perturb-Net preserves stochastic noise that is invariant to the conditioning representation in a single-cell sample, while CPA relies on encoding single-cell data to a basal cellular representation to provide independent stochastic variability from perturbation and covariates for individual cells. The encoded basal cell representation of CPA, however, is very likely to entangle with perturbation and covariates in a one-stage latent-arithmetic modeling framework. In addition, the translations between residual representation and cellular representation of PerturbNet are based on complex normalizing flows, while those of CPA are linear functions which are unable to uncover the complex heterogeneous effects of the same perturbation on different cells.

More importantly, the PerturbNet framework models perturbation responses with dense perturbation representation, while CPA one-hot-encodes perturbation with cell state covariates. The dense perturbation representation of PerturbNet qualifies predicting unobserved perturbations outside of the training data, while labels utilized in CPA constrain the predictions to only the observed perturbations or their combinations. Thus, the functionality of predicting unseen perturbations greatly extends the application scope of PerturbNet.

Experiments

Some embodiments focus on two datasets with chemical perturbations including the sci-Plex (Srivatsan, S. R., et al. (2020), Massively multiplex chemical transcriptomics at single-cell resolution, Science, 367(6473), 45-51) and LINCS data (Subramanian, A., et al. (2017), A next generation connectivity map: L1000 platform and the first 1,000,000 profiles, Cell, 171 (6), 1437-1452). The sci-Plex dataset has scRNA-seq measurements to 188 drug treatments, and LINCS is a microarray dataset with cellular measurements of chemical or genetic perturbations approximately at a single-cell resolution. Thus, some embodiments regard both the sci-Plex data and the LINCS subset with 20,065 chemical perturbations (LINCS-Drug) as single-cell responses to chemical perturbations. Table 1 summarizes the measurement information of the two datasets.

TABLE 1
High-Throughput Gene Expression Datasets
with Chemical Perturbations.

| Dataset | sci-Plex | LINCS-Drug |
|---|---|---|
| Source | scRNA-seq | Microarrays |
| Cell Lines | A549, K562, MCF7 | ~100 |
| Number of Measurements | 648,857 | 689,831 |
| Number of Genes | 5087 | 978 |
| Number of Perturbations | 188 | 20,065 |

Chemical variational autoencoder Gives Meaningful Perturbation Representations

Following one work (Gómez-Bombarelli, R., et al. (2018), Automatic chemical design using a data-driven continuous representation of molecules, ACS central science, 4(2), 268-276), some embodiments trained the chemical variational autoencoder on the ZINC database (Irwin, J. J., and B. K. Shoichet (2005), Zinc- a free database of commercially available compounds for virtual screening, Journal of chemical information and modeling, 45(1), 177-182) with around 250,000 drug treatments. From the trained chemical variational autoencoder, some embodiments obtain the perturbation representations of the drug treatments in the sci-Plex and LINCS-Drug datasets. As the sci-Plex dataset possesses integer count scRNA-seq samples and LINCS-Drug has normally distributed microarray samples, some embodiments obtained their cellular representations by training scVl on the sci-Plex data and VAE on the LINCS data.

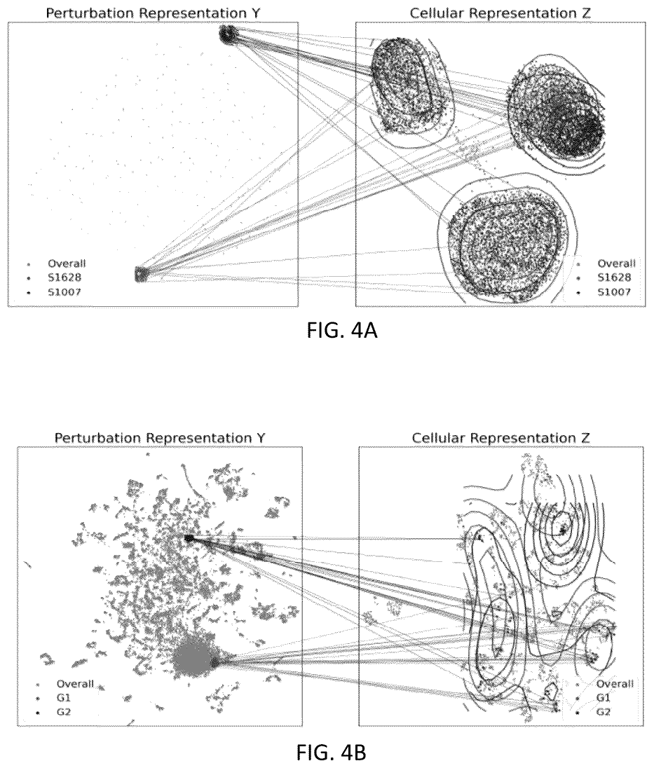

FIG. 4A shows the UMAP plots of the perturbation representations and the cellular representations of two drug treatments of S1628 and S1007 in the sci-Plex data. The two treatments have distinctive perturbation representations and their cell states are also different.

FIG. 4B shows UMAP plots of perturbation representations and cellular representations of two drugs in the LINCS-Drug data.

The drugs of G1 and G2 have very different latent values within the overall perturbation representation space, which map to two unique cell state distributions. Therefore, the perturbation representations from chemical variational autoencoder can reflect the particular perturbation effects on cell states.

KNN Models Have Better Generation than Random Models

Some embodiments implement the baseline models on the sci-Plex and LINCS-Drug data to predict single-cell responses to chemical perturbations. Both baseline KNN and random models predict single-cell perturbation responses by sampling from responses of other perturbations and have relatively consistent performance across perturbations, while PerturbNet tends to have different performances between perturbations utilized to train the model and unobserved perturbations. Therefore, some embodiments partition the set of perturbations to observed perturbations for model training and unseen perturbations. For the KNN model, some embodiments train a KNN graph on the representations of the observed perturbations to select five nearest neighbors for each of the observed and unseen perturbations. To predict responses of each target perturbation in either the observed or unseen set using the random model, some embodiments randomly sampled cells with observed perturbations other than the target perturbation.

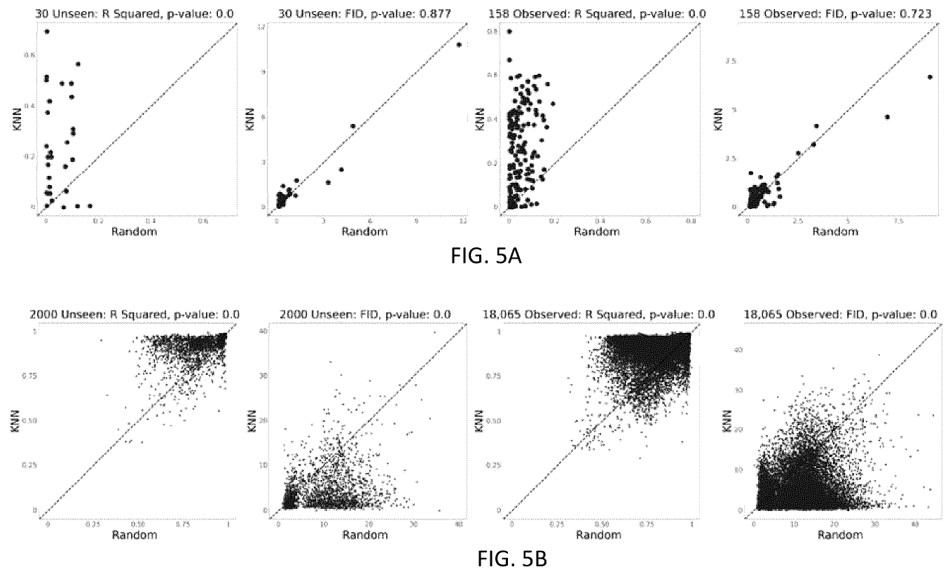

Some embodiments employ the R squared and Fréchet inception distance (FID) metrics to evaluate prediction performance of single-cell responses using the baseline models. A higher R squared and lower FID values better reflect an alignment between the predicted samples and real samples. Some embodiments compare the prediction performances between the KNN model and the random model for both unseen and observed perturbations, with the one-sided Wilcoxon test. FIGS. 5A and 5B show the performance of baseline models for both unseen and observed treatments of the sci-Plex and LINCS-Drug data. More specifically, FIGS. 5A and 5B show R squared and FID of KNN model over random model for unseen and observed drug treatments of the sci-Plex (FIG. 5A) and LINCS-Drug (FIG. 5B) data. For the sci-Plex data, the KNN model has significantly higher R squared values than the random model, while the FID does not give a significant difference between the two baseline models. For the LINCS-Drug data, KNN outperforms the random model in both R squared and FID for either the 2000 unseen or 18,065 observed perturbations.

PerturbNet Predicts Single-Cell Perturbation Responses to Drug Treatments

Some embodiments employ PerturbNet to predict single-cell responses to drug treatments. Some embodiments trained scVl on the sci-Plex count data with 158 observed drug treatments, and VAE on the LINCS-Drug data with 18,065 observed drug treatments. Some embodiments utilize pairs of perturbation and cellular representations of the single-cell subset with observed perturbations to establish the cINN translations of the PerturbNet trained with translations from perturbation to cell state and cellular response.

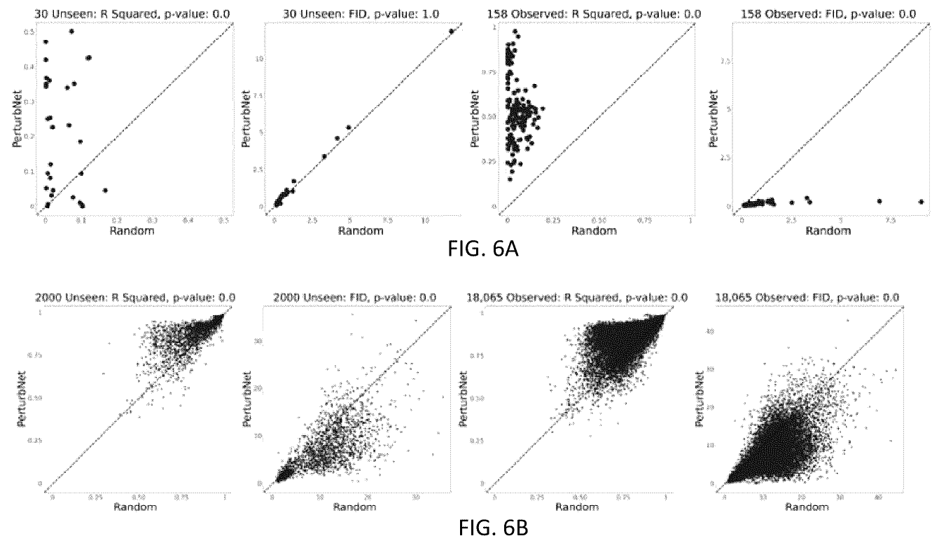

After constructing the PerturbNet framework on the two datasets, some embodiments predict single-cell responses to each of the unseen and observed perturbations. Some embodiments evaluate the performances of PerturbNet for the two datasets and compared them to those of the baseline random model (FIGS. 6A and 6B). As can be seen, PerturbNet has overall better predictions than the baseline random model. Although PerturbNet does not beat the random model in FID for the unseen perturbations of the sci-Plex data, it has significantly lower FID than the random model for the observed perturbations. For the LINCS-Drug data, PerturbNet outperforms the random model with significantly better R squared and FID. The LINCS-Drug dataset has many more perturbations than the sci-Plex data for training the cINN translations of the PerturbNet, and possibly achieves better out-of-sample predictions for unseen perturbations.

The examples of FIGS. 6A and 6B show R squared and FID of PerturbNet over baseline random model for unseen and observed drug treatments of the sci-Plex (FIG. 6A) and LINCS-Drug (FIG. 6B) data.

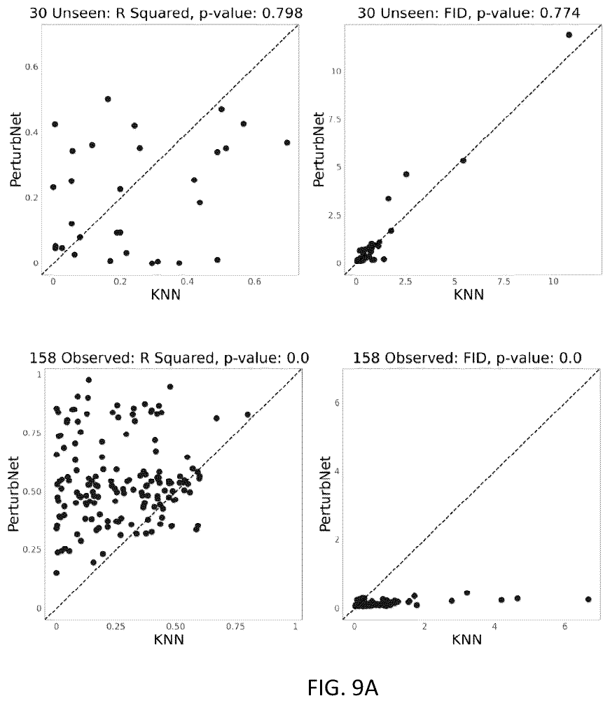

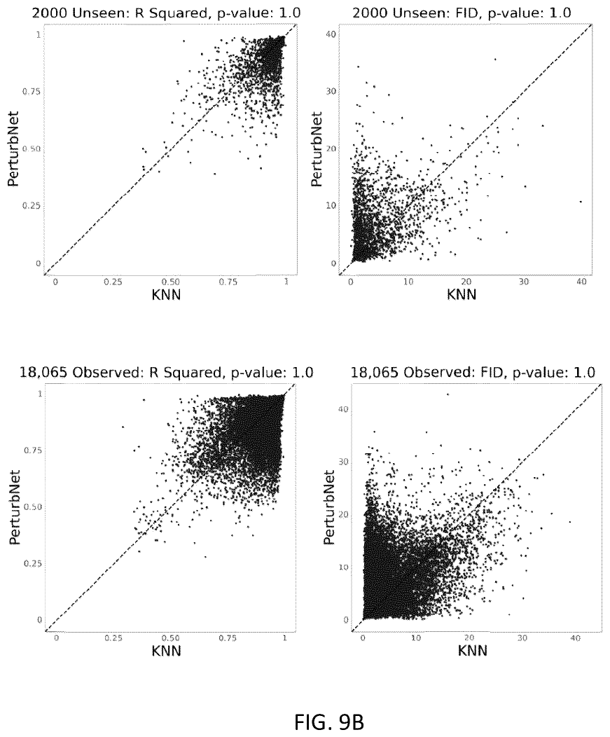

Also compared was the performances of PerturbNet and KNN in FIGS. 9A and 9B. The PerturbNet model has significantly better metric values than the KNN model for the observed perturbations of the sci-Plex data, while it does not exceed the KNN model for unseen perturbations of the sci-Plex, LINCS-Drug, or observed perturbations of the LINCS-Drug. The limited 30 unseen perturbations of the sci-Plex data might be insufficient to significantly distinguish the performances of KNN and PerturbNet. In addition, the LINCS-Drug dataset has a smaller variability with high prediction performances from the random model, and thus might enable the KNN model as a difficult baseline model to be overcome by PerturbNet. In particular, FIG. 9A shows R squared and FID of PerturbNet over baseline KNN model for unseen and observed drug treatments of the sci-Plex data. And FIG. 9B shows R squared and FID of PerturbNet over baseline KNN model for unseen and observed drug treatments of the LINCS-Drug data.

Covariate Adjustment Gives Better Predictions for PerturbNet

As the sci-Plex dataset has two cell state covariates of cell type and dose, some embodiments adjust these covariates in modeling cINN translations of PerturbNet. Some embodiments encode cell type and dose to the one-hot encodings, and concatenat them to the perturbation representation Y as a joint condition representation. Some embodiments then train cINN with the joint representation of perturbation and covariates as conditions for translations between residual representation and cellular representation. Some embodiments then predict single-cell responses to a perturbation with the specific values of covariates.

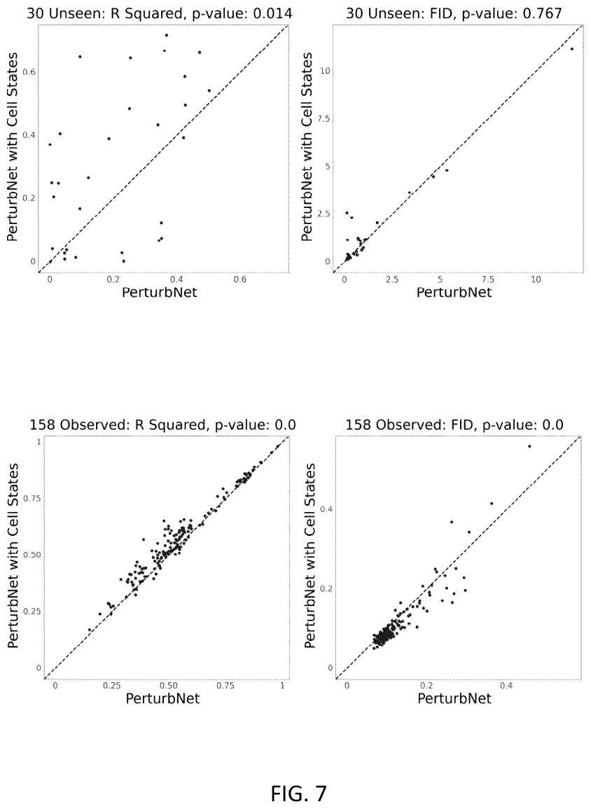

Some embodiments evaluate the prediction performance of PerturbNet adjusted for covariates on the unseen and observed perturbations with cell covariates' values, and compared its performance with that of the previous PerturbNet trained without the cell state covariates. As can be seen in FIG. 7, the PerturbNet adjusted for cell state covariates significantly outperforms the PerturbNet without covariate adjustment for observed perturbations in both R squared and FID. The PerturbNet adjusted for covariates improves R squared for the unseen perturbations. The cell state covariates are correlated with perturbation assignment and also influence cellular responses, making them possess confounding effects in modeling perturbation responses. Therefore, adjusting for covariates in cINN modeling of the PerturbNet helps debias their confounding effects, and more accurately quantify perturbation effects.

More specifically, FIG. 7 shows R squared and FID of PerturbNet adjusted for cell state covariates over the unadjusted PerturbNet for 30 unseen and 158 observed drug treatments of the sci-Plex data.

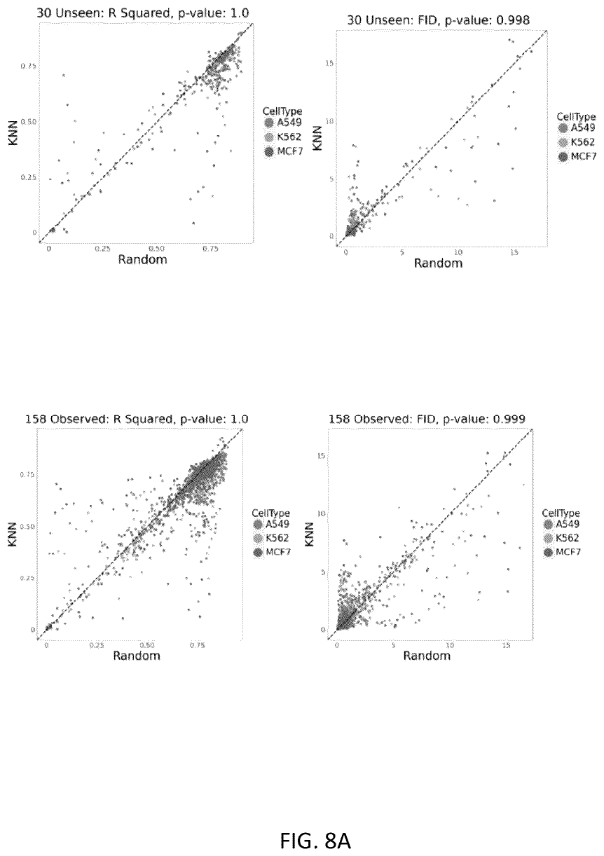

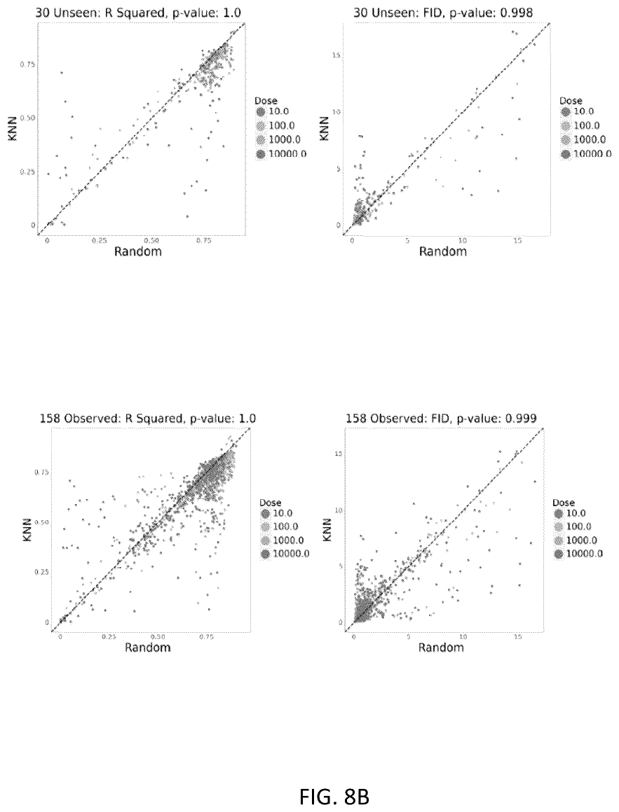

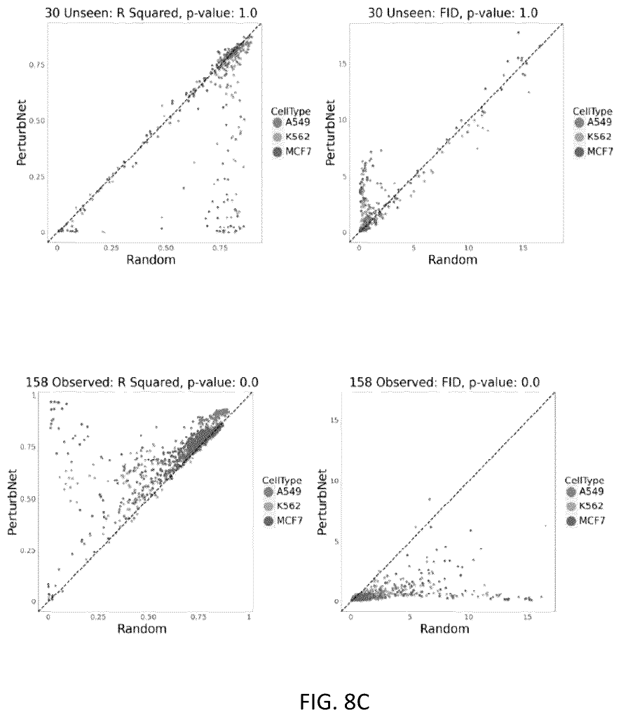

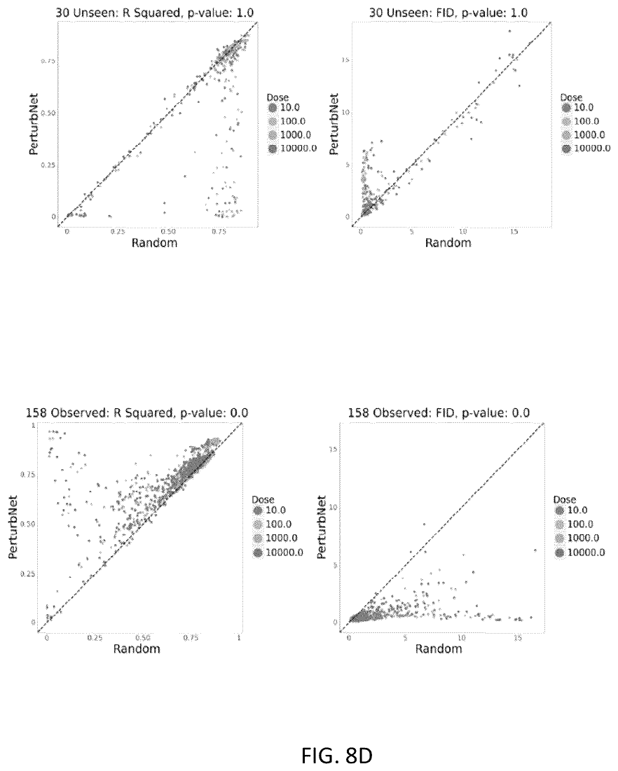

Some embodiments compared the performance of PerturbNet adjusted for covariates with the baseline models. As the PerturbNet adjusted for covariates takes additional covariate information other than perturbation, some embodiments performed a stratified prediction in each cell type by dose stratum to also adjust covariate information for the baseline models. Each perturbation has 12 strata with three cell types and four doses. Some implementations proceeded with the sampling procedures of the baseline KNN and random models within each cell by dose stratum, and made PerturbNet predictions with the corresponding co-variates' values in the stratum. FIGS. 8A-8D show that PerturbNet consistently outperforms the random model for observed perturbations, while KNN is unable to beat the random model for either unseen or observed perturbations. More specifically, FIG. 8A shows R squared and FID of KNN visualized by cell type. FIG. 8B shows R squared and FID of KNN visualized by dose. FIG. 8C shows R squared and FID of PerturbNet adjusted for covariates visualized by cell type. FIG. 8D shows R squared and FID of PerturbNet adjusted for covariates visualized by cell dose. All of FIGS. 8A-8D use the random model for 30 unseen and 158 observed drug treatments in each stratum of cell type by dose of the sci-Plex data.

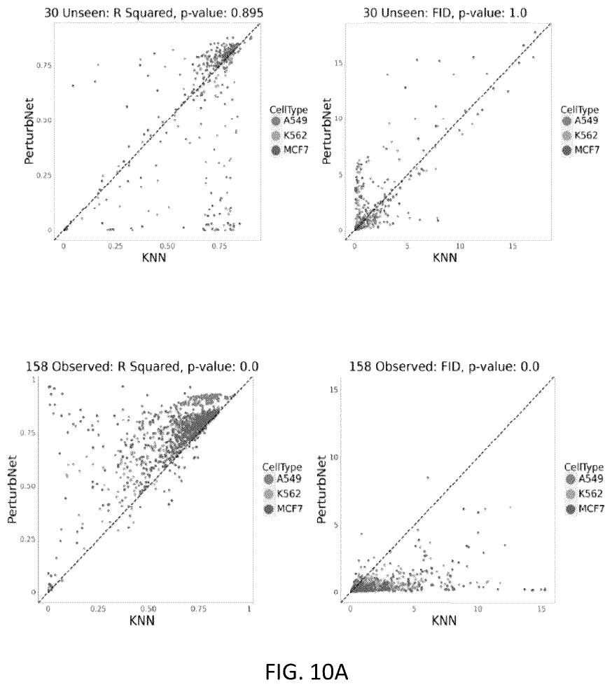

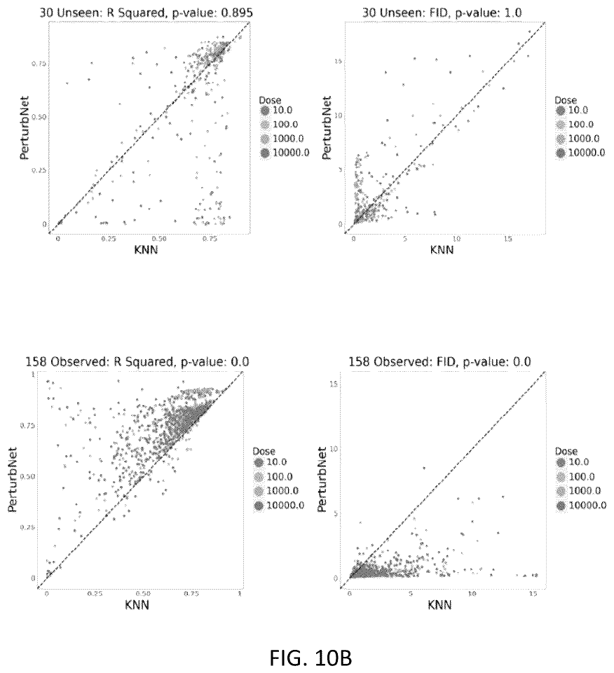

As the stratified evaluations constrain cellular variability and sample size, which possibly narrows down the prediction performances of the KNN and random models, some implementations also compared PerturbNet adjusted for covariates and KNN in stratified predictions (FIGS. 10A and 10B). As with their unstratified comparisons in FIGS. 9A and 9B, the PerturbNet has a better performance for observed perturbations but does not defeat KNN for unseen perturbations. FIG. 10A shows R squared and FID of PerturbNet adjusted for covariates over KNN for 30 unseen and 158 observed drug treatments, visualized by cell type. FIG. 10B shows R squared and FID of PerturbNet adjusted for covariates over KNN for 30 unseen and 158 observed drug treatments, visualized by dose.

Adjusting Confounders of Perturbations in PerturbNet

As discussed above, some embodiments adjusted the covariates in PerturbNet to improve the prediction performance for both unseen and observed perturbations. Some implementations illustrated potential confounding effects of the cell state covariates in modeling perturbations on single-cell responses, and showed that PerturbNet adjusted for covariates achieved better predictions. The following discusses impacts of confounding effects of cell state covariates in learning representations and predicting perturbation responses in the cINN modeling of the PerturbNet.

Some implementations trained VAE on the sci-Plex subset without the three unseen combinations. Some implementations then one-hot-encoded cell type or perturbation, and constructed the cINN translations between one-hot-encoded cell type or one-hot-encoded perturbation and cellular representation. Some implementations then adapted these cINN transitions to perform a similar procedure to latent space vector arithmetic to predict single-cell data of each cell type/treatment combination. For the cINN trained between one-hot-encoded cell type and cellular representation, some implementations utilized a group of control cells of another type and the same treatment to obtain their residual representations V's via the cINN inverse translation, and predict their counterfactual cellular representation and single-cell data through the cINN translation with the cell type of the target combination. This prediction was named cell type translation. For the cINN trained between one-hot-encoded perturbation and cellular representation, some implementations utilized a group of control cells of other treatments and the same cell type to obtain their residual representations V's and predict their counterfactual cellular representation and single-cell data using the perturbation of the target combination through the cINN inverse and forward translations, respectively. This prediction was named as treatment translation. The two predictions assumed that the residual representations V's in the cell type cINN translations preserved perturbation information to predicted counterfactual cellular representation, and vice versa.

Some implementations also trained a PerturbNet model on the sci-Plex subset without the three unseen combinations with the cINN translations between concatenated one-hot-encoded representations of perturbation, cell type and dose. Some implementations then predicted each combination by generating cells from PerturbNet. In addition to cell type translation, treatment translation and PerturbNet, some implementations also predicted each combination using the latent space vector arithmetic algorithm.

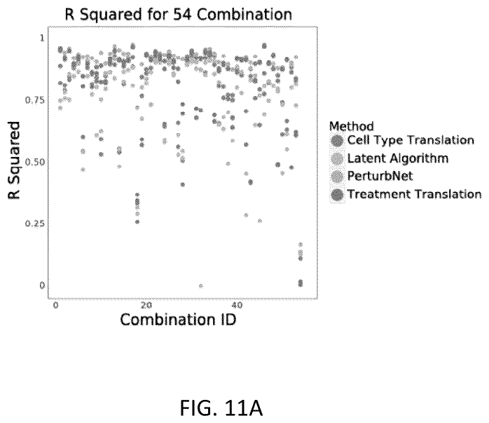

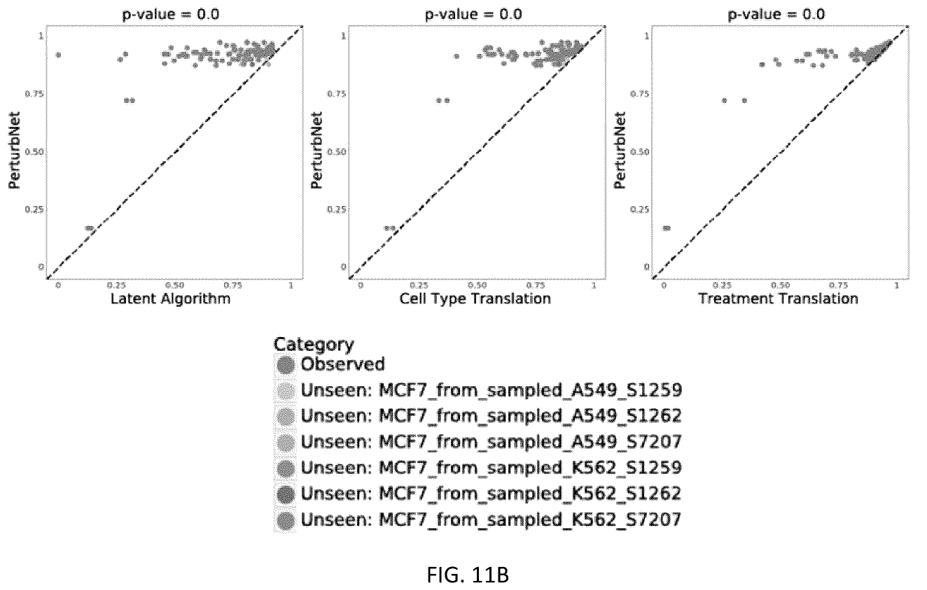

Some implementations evaluated the predicted data using the R squared metric and show the R squared values of the four methods across the 54 combinations (FIG. 11A). PerturbNet has the highest R squared among the four methods for almost all the combinations. FIG. 11B also shows that PerturbNet predictions has significantly higher R squared than latent space vector arithmetic, cell type translation and treatment translation. It also has higher R squared for the three unseen combinations.

More particularly, FIG. 11A shows R squared of predictions of cell type/treatment combinations using latent space vector arithmetic (latent algorithm), cell type translation, treatment translation and PerturbNet. FIG. 11B shows R squared of predicted cell type/treatment combinations between PerturbNet and each of latent algorithm, cell type translation and treatment translation. The p-values are from the one-sided Wilcoxon test.

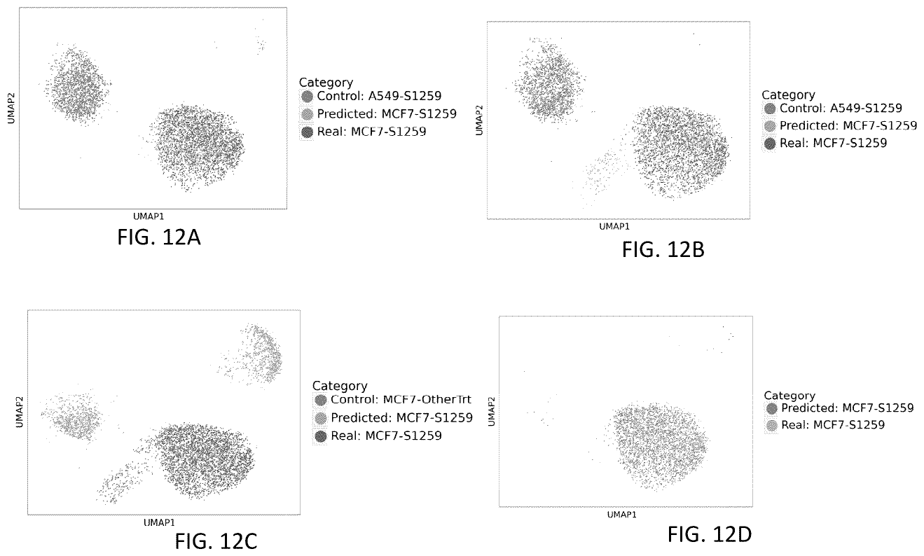

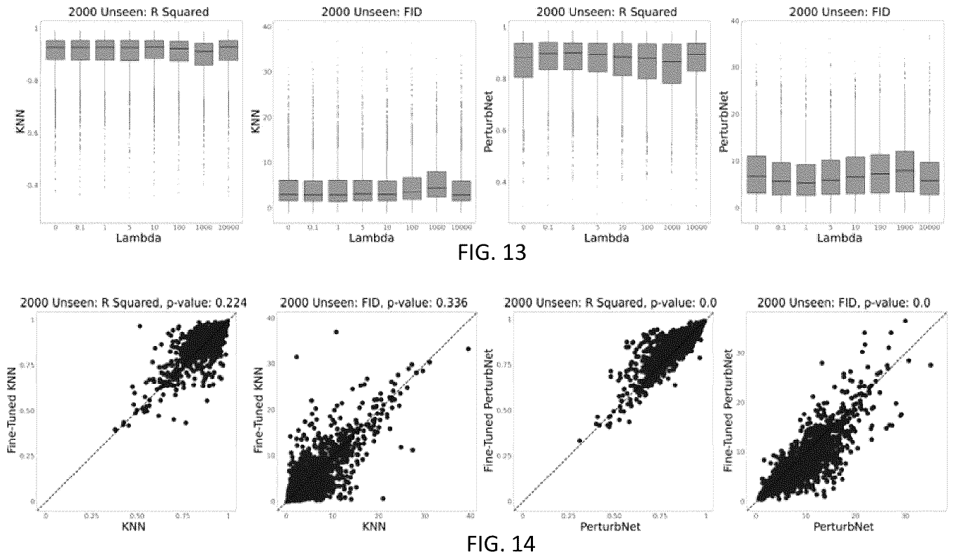

FIGS. 12A-12D show the UMAP plots of predicted MCF7-S1259 unseen combination from the four methods. The latent space vector arithmetic and PerturbNet generally recover the real cells of the combination, while cell type translation generate cells of perturbations other than S1259 and treatment translation gives cells of cell types other than MCF7. This means that the residual representations V's in the cINN trained with only cell type, and no treatment, does not preserve the treatment information, nor did the residual representations in the cINN trained with only drug treatment, no cell type, fail to preserve the cell type. The PerturbNet fully adjusted for perturbation and covariates has the best predicted cells of MCF7-S1259, which overlap well with the real cells.

More specifically, FIGS. 12A-12D show: UMAP plots of predicted MCF7-S1259 using latent space vector algorithm (FIG. 12A), cell type translation (FIG. 12B) treatment translation (FIG. 12C) and PerturbNet (FIG. 12D).

Therefore, both cell type translation and treatment translation have a more parsimonious modeling procedure and do not need to adjust for other covariates in their cINN models, but their residual representations might not preserve meaningful information for individual cells. A possible reason for their entangled residual representations is that a cINN model assumes independence between condition and residual representation, while cell type is actually correlated with perturbation assignment in the sci-Plex subset. Thus, the residual representation does not carry meaningful perturbation or cell type information in the two translations for an individual cell. On the other hand, modeling cell type/treatment combinations using PerturbNet requires specifying covariates in the cINN modeling, and these covariates help make more unbiased predictions.

Fine-Tuned chemical variational autoencoder Improves Perturb-Net Performance

Some implementations performed chemical variational autoencoder fine-tuning to improve the performance of Perturb-Net. To construct a cell-state Laplacian matrix L, some embodiments computed the Wasserstein-2 (W2) distance between cellular latent values of each pair of drug treatments. As the number of treatment pairs is extremely large in LINCS-Drug, some embodiments first fitted a KNN algorithm on the perturbation representations and selected the 30 nearest neighbors for each drug treatment to compute their pairwise cellular latent distances. As the resulting pairwise cell latent distance matrix for all the 20,065 treatments was not symmetric, some implementations took the average of the matrix and its transposed matrix. Some implementations then calculated the exponential of their opposite values and row-normalized the matrix to obtain the adjacency matrix with each entry as a transition probability. Some implementations then obtained the Laplacian matrix from the adjacency matrix.

Some implementations utilized the Laplacian sub-matrix for the observed drug treatments of LINCS-Drug to fine-tune chemical variational autoencoder. Some embodiments considered values of λ in (0.1, 1, 5, 10, 100, 1000, 10,000) to implement the chemical variational autoencoder fine-tuning algorithm. Subsequent to fine-tuning the chemical variational autoencoder, some implementations evaluated the KNN model on the perturbation representations from these fine-tuned chemical variational autoencoder. Some implementations also constructed the cINN model of the PerturbNet between the perturbation representations of the fined-tuned chemical variational autoencoder and cellular representations using cells with the observed perturbations. Some implementations evaluated the prediction performance of the fine-tuned KNN and PerturbNet models on the 2000 unseen perturbations of the LINCS-Drug data (FIG. 13). Both R squared and FID of PerturbNet have small to medium fluctuations across increasing λ values, while those of KNN do not obviously change with varying values. Several λ values give slight increases of median R squared or decreases of median FID for PerturbNet over the non-fine-tuned one, such as λ=0.1, 1, 5, 10, 100.

More specifically, FIG. 13 shows example R squared and FID of KNN and PerturbNet with fine-tuned Chemical-VAE across different λ values for the 2000 unseen drug treatments of the LINCS-Drug data.

FIG. 14 shows an example comparison of the fine-tuned KNN and PerturbNet with λ=1 to their non-fine-tuned counterparts for the unseen perturbations. The fine-tuned PerturbNet has significant improvements in both R squared and FID, while fine-tuning the chemical variational autoencoder does not significantly enhance KNN. A possible explanation is that the cINN of PerturbNet further enforces the prediction capacity from fine-tuned perturbation representation to cell state.

PerturbNet Recovers the Perturbation and Cell Latent Spaces

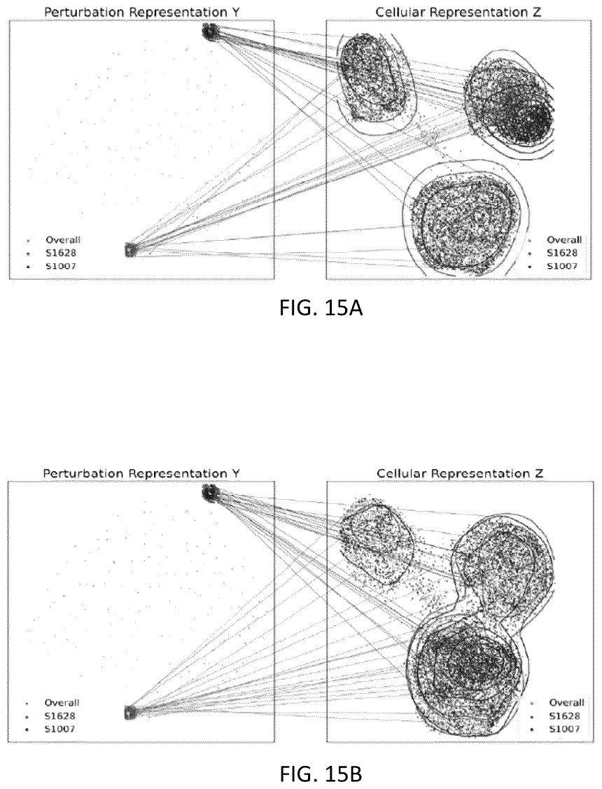

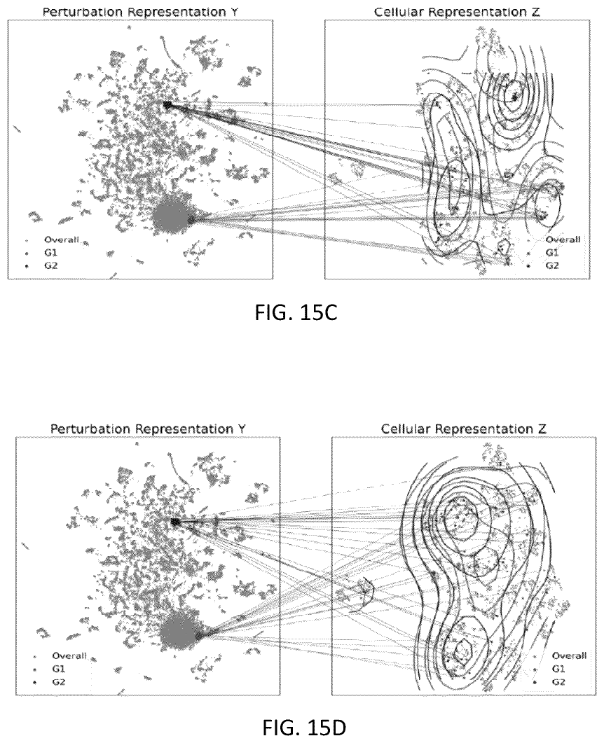

Some implementations used PerturbNet to generate cellular representations from the perturbation representation of a drug treatment. Here, reconstructing cellular representations was considered using V's inferred from real cells, or sampling cellular representations using V's from the prior distribution. The perturbations of the sci-Plex and LINCS-Drug data in FIGS. 4A-4B were used to reconstruct and sample cellular representations. The UMAP plots of these cellular representations are shown in FIGS. 15A-15D. As can be seen, the reconstructed representations recover the observed cellular representations of both the sci-Plex and LINCS-Drug data in FIGS. 4A-4B, while the sampled representations do not strictly reflect the observed latent distributions. The residual representations possess condition-invariant information to reconstruct individual cells, and those sampled from the prior distribution generate general cell states of a perturbation.

Therefore, the predicted single-cell responses to a perturbation via PerturbNet by sampling V˜ [symbol] (0,1) does not specifically recover the observed individual cells to the perturbation, as they possess specific residual information. PerturbNet may predict single-cell perturbation responses with a general residual distribution, which maps to the overall training data in the cINN modeling of PerturbNet. If prior information about the individual residual representation is available, more precise predictions can be made on individual perturbation responses for observed cells, especially for unseen perturbations. However, cells are usually measured and destroyed in single-cell experiments before this residual information can be inferred.

### DISCUSSION

The techniques disclosed herein propose a deep generative model, PerturbNet, to predict single-cell responses to chemical perturbations. Some implementations encode cell samples and drug SMILES strings to dense latent representations using single-cell VAE and chemical variational autoencoder. Some implementations then connect two representations through cINN. PerturbNet gives a stable training process, which has two stages of chemical variational autoencoder and single-cell VAE trained separately and integrated through cINN. The PerturbNet framework can make predictions for both unseen and observed drug treatments. Some implementations perform experiments to show that PerturbNet has excellent prediction performance for single-cell responses to both unseen and observed drug treatments. In addition, the disclosed chemical variational autoencoder fine-tuning algorithm also improves the prediction performance using fine-tuned perturbation representation.

The CPA framework also predicts single-cell perturbation responses to chemical perturbations (Lotfollahi, M., A. K. Susmelj, C. De Donno, Y. Ji, I. L. Ibarra, F. A. Wolf, N. Yakubova, F. J. Theis, and D. Lopez-Paz (2021), Compositional perturbation autoencoder for single-cell response modeling, bioRxiv). The CPA framework also utilizes a single-cell VAE and models perturbation effects on latent values of cells. However, the encoder of CPA only gives a basal cell state, while perturbation is further incorporated to decode to single-cell data, making it a very unusual autoencoder framework lying between VAEs and conditional VAEs. In addition, the latent linear model in CPA takes drug treatments as categories, and thus cannot make predictions for unseen drug treatments. Therefore, some embodiments do not directly compare CPA and PerturbNet due to their different functionalities.

A limitation of the experiments may be with the limited number of chemical perturbations. This disclosure finds that LINCS-Drug has many more chemical perturbations and a slightly better prediction performance for unseen perturbations than the sci-Plex data. Having more chemical perturbations enables more powerful cINN connections of the PerturbNet to infer the cellular representation for each unseen perturbation representation. Therefore, future research can implement PerturbNet on new high-throughput chemical screen datasets with many more drug treatments.

Another future improvement of PerturbNet is to train chemical variational autoencoder with larger chemical databases, such as PubChem (Kim, S., et al. (2016), Pubchem substance and compound databases, Nucleic acids research, 44(D1), D1202-D1213) with millions of drug treatments. In addition, having better preprocessing steps of chemical SMILES strings might further improve the training of chemical variational autoencoder to obtain better perturbation representations. Other advanced deep generative models for chemical perturbations can also be employed to replace the chemical variational autoencoder model of the PerturbNet. For example, one work (Jin, W., R. Barzilay, and T. Jaakkola (2020a), Hierarchical generation of molecular graphs using structural motifs, in International Conference on Machine Learning, pp. 4839-4848, PMLR) proposed a hierarchical VAE structure for graph generation of molecules and multi-layer representations of drug treatments. These multi-resolution perturbation representations can potentially give better single-cell predictions to unseen drug treatments.

Datasets

Some implementations use the ZINC database with 250,000 compounds (Irwin, J. J., and B. K. Shoichet (2005), Zinc- a free database of commercially available compounds for virtual screening, Journal of chemical information and modeling, 45(1), 177-18) from the chemical variational autoencoder model (https://github.com/aspuru-guzik-group/chemical_vae/tree/main/models/zinc). Some implementations transformed the compounds to canonical SMILES following the chemical variational autoencoder tutorial (https://github.com/aspuru-guzik-group/chemical_vae/blob/main/examples/intro_to_chemvae.ipynb) via the RDKit package (Landrum, G. (2016), Rdkit: open-source cheminformatics http://www.rdkit. org, Google Scholar). Some implementations also utilized the chemical elements' library from this tutorial to define the one-hot matrices of drug treatments, where the maximum length of canonical SMILES strings was constrained to be 120.

Some implementations processed the whole sci-Plex data (Srivatsan, S. R., et al. (2020), Massively multiplex chemical transcriptomics at single-cell resolution, Science, 367(6473), 45-51) using SCANPY (Wolf, F. A., P. Angerer, and F. J. Theis (2018), Scanpy: large-scale single-cell gene expression data analysis, Genome biology, 19(1), 15) with a lot of 648,857 cells and 5087 genes. There were 634,110 cells perturbed by 188 drug treatments in total, with 14,627 cells treated by unknown drug treatments and 120 unperturbed cells. Some implementations randomly selected 30 drug treatments as unseen perturbations and the other 158 drug treatments as observed perturbations.

Some implementations obtained the LINCS dataset (Subramanian, A., et al. (2017), A next generation connectivity map: L1000 platform and the first 1,000,000 profiles, Cell, 171 (6), 1437-1452) from GEO accession ID GSE92742. The LINCS data had been processed with 1,319,138 cells and 978 landmark genes, containing the LINCS-Drug subset with 689,831 cells treated by 20,329 drug treatments denoted with their SMILES, 20,065 drug treatments of which had lengths smaller than 120. Some implementations randomly selected 2000 drug treatments as unseen perturbations and the other 18,065 drug treatments as observed perturbations. Some implementations transformed the SMILES strings of drug treatments of the sci-Plex and LINCS-Drug data to their one-hot matrices according to the chemical elements' library.

Prediction Metrics—R Squared

Some implementations follow the R Squared metric utilized in several frameworks to predict single-cell responses to perturbations (Lotfollahi, M., F. A. Wolf, and F. J. Theis (2019), scgen predicts single-cell perturbation responses. Nature methods, 16(8), 715-721; Lotfollahi, M., M. Naghipourfar, F. J. Theis, and F. A. Wolf (2020), Conditional out-of-distribution generation for unpaired data using transfer vae, Bioinformatics, 36(Supplement 2), i610-i617; Lotfollahi, M., A. K. Susmelj, C. De Donno, Y. Ji, I. L. Ibarra, F. A. Wolf, N. Yakubova, F. J. Theis, and D. Lopez-Paz (2021), Compositional perturbation autoencoder for single-cell response modeling, bioRxiv). Some implementations first obtain the normalized data of predicted and real single-cell responses to a perturbation for the sci-Plex data. Some implementations conduct similar processing steps to SCANPY (Wolf, F. A., P. Angerer, and F. J. Theis (2018), Scanpy: large-scale single-cell gene expression data analysis, Genome biology, 19(1), 15). Some implementations first normalize the total number of counts of each cell to be 10⁴, take log-transformation, and scale the values. Some implementations directly use LINCS samples as they have already been normalized. Some implementations compute the mean gene expression values of normalized data of both predicted and real cells to a drug treatment. Some implementations then fit a simple linear regression model on the real mean gene expression values over the predicted mean gene expression values. The R squared of the fitted linear regression is then reported to quantify the accuracy of predicted cells.

Prediction Metrics—FID Score

Some implementations define an FID score metric similar to the FID metric utilized in image data (Heusel, M., H. Ramsauer, T. Unterthiner, B. Nessler, and S. Hochreiter (2017), Gans trained by a two time-scale update rule converge to a local nash equilibrium, in Advances in neural information processing systems, pp. 6626-6637). Some implementations train a single-cell VAE model on the whole single-cell dataset using either scVl or VAE depending on the data type. Some implementations obtain the cell latent values of the predicted and real cells to a perturbation. Some implementations then apply the Fréchet distance to the latent values of predicted and real cells with the Gaussian assumption

FID=∥μ_{Real}−μ_{Predicted}∥₂²+trace{Σ_{Real}+Σ_{Predicted}−2(Σ_{Real}Σ_{Predicted)}^{1/2}},

where μ_{Real}, μ_{Predicted} are means of predicted and real latent values, and Σ_{Real}, Σ_{Predicted} are covariance matrices of predicted and real latent values.

Predicting Single-Cell Responses to Genetic Perturbations—Introduction

Unlike chemical perturbations, whose direct gene targets are generally unknown, genetic perturbations are designed to directly knock out or activate one or several target genes. The genes' activation or knockout will not only influence their own expression, but also impact other genes through a complex network of downstream gene regulatory interactions. The Clustered Regularly Interspaced Short Palindromic Repeats (CRISPR) technology allows an easy design of precise genetic mutants through genome editing (Doudna, J. A., and E. Charpentier (2014), The new frontier of genome engineering with crispr-cas9, Science, 346(6213)). More recently, CRISPR has been combined with transcriptional activators (CRISPRa) or repressors (CRISPRi) tethered to dCas9 to enable activation or inhibition of target genes. The Perturb-seq technology combines CRISPR gene editing and single-cell RNA-sequencing (scRNA-seq) to measure single-cell responses to pooled CRISPR screens (Dixit, A., et al. (2016), Perturb-seq: dissecting molecular circuits with scalable single-cell rna profiling of pooled genetic screens, cell, 167(7), 1853-1866). Perturb-seq measures cellular responses at single-cell resolution, which reveals how cell states are impacted by genetic perturbations, and has been utilized for many biomedical applications (Wang, T., K. Birsoy, N. W. Hughes, K. M. Krupczak, Y. Post, J. J. Wei, E. S. Lander, and D. M. Sabatini (2015), Identification and characterization of essential genes in the human genome, Science, 350(6264), 1096-1101; Adamson, B., et al. (2016), A multiplexed single-cell crispr screening platform enables systematic dissection of the unfolded protein response, Cell, 167(7), 1867-1882; Datlinger, P., et al. (2017), Pooled crispr screening with single-cell transcriptome readout, Nature methods, 14(3), 297-301; Ursu, O., et al. (2020), Massively parallel phenotyping of variant impact in cancer with perturb-seq reveals a shift in the spectrum of cell states induced by somatic mutations, bioRxiv; Jin, X., et al. (2020b), In vivo perturb-seq reveals neuronal and glial abnormalities associated with autism risk genes, Science, 370(6520)). However, as Perturb-seq experiments can only measure limited numbers of perturbation loci, while the human genome contains about 3.2 billion nucleotides of DNA (Brown, T. A. (2018), Genomes 4, Garland science), it is not feasible to measure single-cell responses for each potential genetic perturbation.

Various methods have been proposed to detect perturbations that have interpretable effects on cellular responses to genetic perturbations. One work (Norman, T. M., M. A. Horlbeck, J. M. Replogle, Y. G. Alex, A. Xu, M. Jost, L. A. Gilbert, and J. S. Weissman (2019), Exploring genetic interaction manifolds constructed from rich single-cell phenotypes, Science, 365(6455), 786-793) used Perturb-seq data to identify genetic interaction from paired gene knockouts. Another work (Burkhardt, D. B., et al. (2021), Quantifying the effect of experimental perturbations at single-cell resolution, Nature biotechnology, 39(5), 619-629) identified perturbation effects over the cellular manifold, using graph signal processing tools. Yet another work (Yeo, G. H. T., S. D. Saksena, and D. K. Gifford (2021), Generative modeling of single-cell time series with prescient enables prediction of cell trajectories with interventions, Nature communications, 12(1), 1-12) proposed a generative model using a diffusion process over a potential energy landscape to learn the underlying differentiation landscape from time-series scRNA-seq data and to predict cellular trajectories under perturbations. Linear models were also used to estimate the impact of perturbations on high-dimensional scRNA-seq data (Dixit, A., et al. (2016), Perturb-seq: dissecting molecular circuits with scalable single-cell rna profiling of pooled genetic screens, cell, 167(7), 1853-1866) or infer gene regulatory networks (GRNs) from perturbations (Kamimoto, K., C. M. Hoffmann, and S. A. Morris (2020), Celloracle: Dissecting cell identity via network inference and in silico gene perturbation, bioRxiv). However, these linear models had limited predictive power on the non-linear gene expression profiles of perturbations and other cell state variables. One related work is the compositional perturbation autoencoder (CPA) framework (Lotfollahi, M., A. K. Susmelj, C. De Donno, Y. Ji, I. L. Ibarra, F. A. Wolf, N. Yakubova, F. J. Theis, and D. Lopez-Paz (2021), Compositional perturbation autoencoder for single-cell response modeling, bioRxiv), which generates single-cell data under perturbations based on latent space linear models. However, as with drug treatment perturbations, CPA assumes that the effects of genetic perturbations can be estimated independently of a basal cell state. In addition, although CPA can make predictions on new combinations of observed target genes, it cannot predict single-cell responses to genetic perturbations with unseen target genes.

Therefore, this disclosure develops a deep generative model that predicts single-cell responses to combinatorial genetic perturbations, including both observed and unseen target genes. To do this, some embodiments extend the PerturbNet model using a neural network that learns representations of genetic perturbations. This disclosed network can embed the two main classes of genetic perturbations: genome edits made with CRISPR/Cas9 (Dixit, A., et al. (2016), Perturb-seq: dissecting molecular circuits with scalable single-cell rna profiling of pooled genetic screens, cell, 167(7), 1853-1866) and gene knockdowns or activations using CRISPRi or CRISPRa (Adamson, B., et al. (2016), A multiplexed single-cell crispr screening platform enables systematic dissection of the unfolded protein response, Cell, 167(7), 1867-1882). This genetic perturbation autoencoder allows to translate from the perturbation latent space to the cell state space, then to generate realistic single-cell gene expression profiles from the cell state space.

Methods

Some implementations extend the PerturbNet framework to genetic perturbations by constructing an autoencoder for the target genes in combinatorial genetic perturbations. There are two main types of genetic perturbations. The genetic perturbations using CRISPR activation (CRISPRa) or CRISPR interference (CRISPRi) do not change the original DNA sequence (Norman, T. M., M. A. Horlbeck, J. M. Replogle, Y. G. Alex, A. Xu, M. Jost, L. A. Gilbert, and J. S. Weissman (2019), Exploring genetic interaction manifolds constructed from rich single-cell phenotypes, Science, 365(6455), 786-793). In contrast, CRISPR/Cas9 directly modifies the DNA sequence, leading to changes in the protein-coding sequence or non-coding regulatory sequence. A genetic perturbation can be represented by either the identities of its target genes (Dixit, A., et al. (2016), Perturb-seq: dissecting molecular circuits with scalable single-cell rna profiling of pooled genetic screens, cell, 167(7), 1853-1866) or the final sequence induced by genome editing. For example, one work (Ursu, O., et al. (2020), Massively parallel phenotyping of variant impact in cancer with perturb-seq reveals a shift in the spectrum of cell states induced by somatic mutations, bioRxiv) performed Perturb-seq to assess the impacts of single amino acid changes in the proteins TP53 and KRAS. In this experiment, a TP53 variant of ‘Q5R’ means that the fifth amino acid of the TP53 protein sequence was changed from ‘Q’ to ‘R’. Some implementations construct two types of genetic perturbation autoencoders—one for each type of perturbation representation (target gene identities or edited sequences).

Genetic Perturbations and GenotypeVAE

Some embodiments first consider genetic perturbations represented by target genes. Most of the existing methods one-hot-encode the target genes across a set of genes (Dixit, A., et al. (2016), Perturb-seq: dissecting molecular circuits with scalable single-cell rna profiling of pooled genetic screens, cell, 167(7), 1853-1866), Perturb-seq: dissecting molecular circuits with scalable single-cell rna profiling of pooled genetic screens, cell, 167(7), 1853-1866) or all genes on a coding sequence (Ma, J., M. K. Yu, S. Fong, K. Ono, E. Sage, B. Demchak, R. Sharan, and T. Ideker (2018), Using deep learning to model the hierarchical structure and function of a cell, Nature methods, 290-298). However, this strategy cannot generalize to perturbations with an unseen target gene.

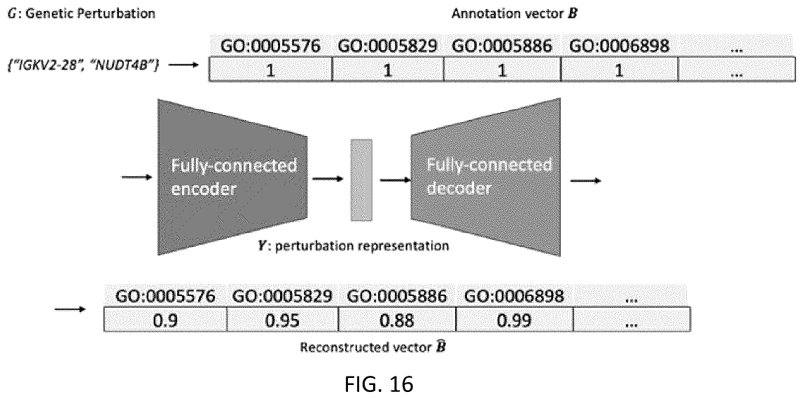

To encode genetic perturbations, some embodiments use a more parsimonious framework and refer to it as GenotypeVAE (e.g., as in the example of FIG. 16). One insight is that the numerous functional annotations of each gene (organized into a hierarchy in the gene ontology) provide features for learning a low-dimensional representation of individual genes and groups of genes. Using gene ontology (GO) terms, some embodiments can represent each target gene g as a one-hot vector B_{g}, where 1's in the vector element correspond to a particular term indicating that the gene has the annotation. One aspect is inspired by a previous work (Chicco, D., P. Sadowski, and P. Baldi (2014), Deep autoencoder neural networks for gene ontology annotation predictions, in Proceedings of the 5th ACM conference on bioinformatics, computational biology, and health informatics, pp. 533-540). If there is a genetic perturbation with multiple target genes {g₁ . . . gₖ}, annotation-wise union operations may be used to generate a one-hot annotation vector for the genetic perturbation as follows:

B_{g1, . . . ,gk}=∪ⱼ₌₁^{k}B_{g}_{ⱼ}

Then, some implementations may train GenotypeVAE using one-hot representations of many possible genetic perturbations. Some implementations use the GO Consortium gene ontology annotation dataset of human genes. This resource annotates 18,832 genes with 15,988 annotation terms (after removing some annotations with insufficient information). Some implementations take the 15,988-dimensional annotation vector as the input to the GenotypeVAE encoder consisting of two hidden layers with 512 and 256 neurons, following output layers for means and standard deviations, both with 10 neurons. The GenotypeVAE decoder also has two hidden layers with 256 and 512 neurons, along with an output layer of 15,988 neurons activated by the sigmoid activation function. Some implementations also have a batch normalization layer, Leaky ReLU activation and a dropout layer with a dropout probability of 0.2 following each hidden layer of GenotypeVAE.

Protein Perturbations and ESM

Some implementations also extend the PerturbNet framework to predict single-cell responses to protein-coding sequence variants. A coding variant can be uniquely represented by the protein sequence resulting from the nucleotide alterations induced by CRISPR/Cas9 editing. Similar to chemical perturbations, protein perturbations can also be summarized as sequences of strings. A key difference is that each character of a protein sequence is a naturally occurring character sequence, whereas a chemical structure is actually a three-dimensional structure (even if it is sometimes represented as a string).

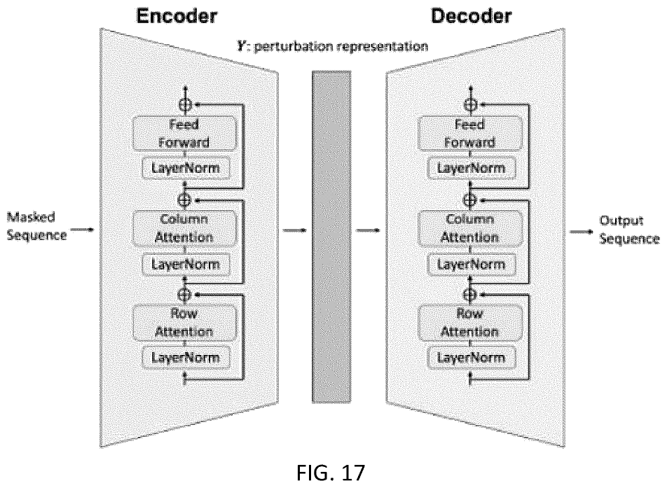

Some embodiments therefore consider a state-of-the-art language model for protein sequences. Some embodiments employ the previously published Evolutionary Scale Modeling (ESM) (Rives, A., et al. (2021), Biological structure and function emerge from scaling un-supervised learning to 250 million protein sequences, Proceedings of the National Academy of Sciences, 118(15)) architecture shown in FIG. 17. ESM is a self-supervised transformer model (Devlin, J., M.-W. Chang, K. Lee, and K. Toutanova (2018), Bert: Pre-training of deep bidirectional transformers for language understanding, arXiv preprint arXiv:1810.04805) and was previously shown to achieve better representations and prediction performance on protein sequences compared to other language models such as long short-term memory (LSTM) networks. As with other transformer models (Vaswani, A., N. Shazeer, N. Parmar, J. Uszkoreit, L. Jones, A. N. Gomez, L. Kaiser, and I. Polosukhin (2017), Attention is all you need, Advances in neural information processing systems, 30), the ESM model was pre-trained on large protein sequence datasets (Rao, R. M., J. Liu, R. Verkuil, J. Meier, J. Canny, P. Abbeel, T. Sercu, and A. Rives (2021), Msa transformer, in International Conference on Machine Learning, pp. 8844-8856, PMLR). Some implementations adopt a pre-trained ESM model specialized for prediction of single variant effects (Meier, J., R. Rao, R. Verkuil, J. Liu, T. Sercu, and A. Rives (2021), Language models enable zero-shot prediction of the effects of mutations on protein function, Advances in Neural Information Processing Systems, 34), because this application is most similar to scenarios in some embodiments.

However, the representation obtained from ESM is deterministic for a given protein sequence. The fixed protein representations limit the amount of training data available for PerturbNet, especially when there is a small number of protein sequences. Some embodiments therefore add low-variance noise Σ to the ESM representation Y_{ESM} from ESM. The final perturbation representation is thus computed as

Y=Y_{ESM}+∈

where ∈˜ [symbol] (0, σ²l). Some embodiments choose the variance σ² to be a positive constant small enough that it does not significantly alter the relative distances between proteins in the ESM latent space.

Experiments

Some implementations applied different prediction methods to several datasets with genetic perturbation. Some implementations used the genetic CRISPR screen data to explore genetic interaction manifolds (GI) (Norman, T. M., M. A. Horlbeck, J. M. Replogle, Y. G. Alex, A. Xu, M. Jost, L. A. Gilbert, and J. S. Weissman (2019), Exploring genetic interaction manifolds constructed from rich single-cell phenotypes, Science, 365(6455), 786-793), LINCS data with 4,109 genetic perturbations (Subramanian, A., et al. (2017), A next generation connectivity map: L1000 platform and the first 1,000,000 profiles, Cell, 171 (6), 1437-1452), genome-scale Perturb-seq data with 9499 perturbations (GSPS) (Replogle, J. M., et al. (2021), Mapping information-rich genotype-phenotype landscapes with genome-scale perturb-seq, bioRxiv), as well as the Perturb-seq data with 1338 coding-sequence variants (Ursu, O., et al. (2020), Massively parallel phenotyping of variant impact in cancer with perturb-seq reveals a shift in the spectrum of cell states induced by somatic mutations, bioRxiv). The first three datasets have genetic perturbations of target gene identities, and the Ursu dataset has genetic perturbations of protein-coding sequence variants. The LINCS is a microarray dataset with cellular measurements approximately at a single-cell resolution, and some implementations used its subset treated by genetic perturbations (LINCS-Gene) as single-cell responses. Table 2 summarizes the measurement information of the datasets, and details of their data preprocessing steps are provided elsewhere herein.

TABLE 2
High-Throughput Gene Expression Datasets
with Genetic Perturbations.

| Dataset | GI | LINCS-Gene | GSPS | Ursu et al. |
|---|---|---|---|---|
| Source | scRNA-seq | Microarrays | scRNA-seq | scRNA-seq |
| Cell Lines | K562 | ~100 | K562 | A549 |
| Number of | 109,738 | 442,684 | 1,989,373 | 164,931 |
| Measurements | | | | |
| Number of | 2279 | 978 | 2000 | 1629 |
| Genes | | | | |
| Number of | 230 | 4109 | 9499 | 1338 |
| Perturbations | | | | |
| Perturbation | Gene | Gene | Gene | Sequence |
| Identity | | | | |

PerturbNet Models Latent Representations of Genetic Perturbations

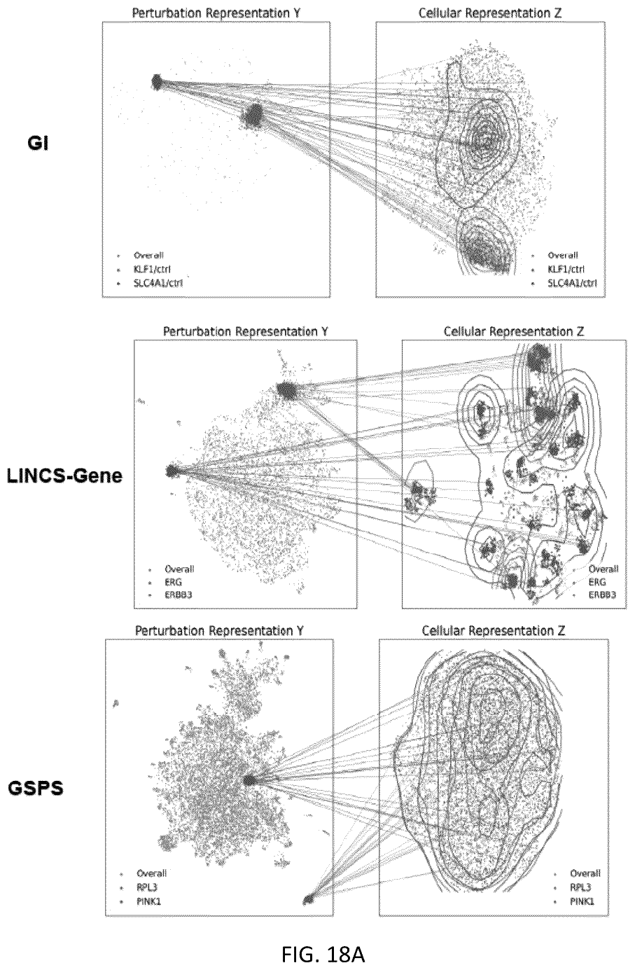

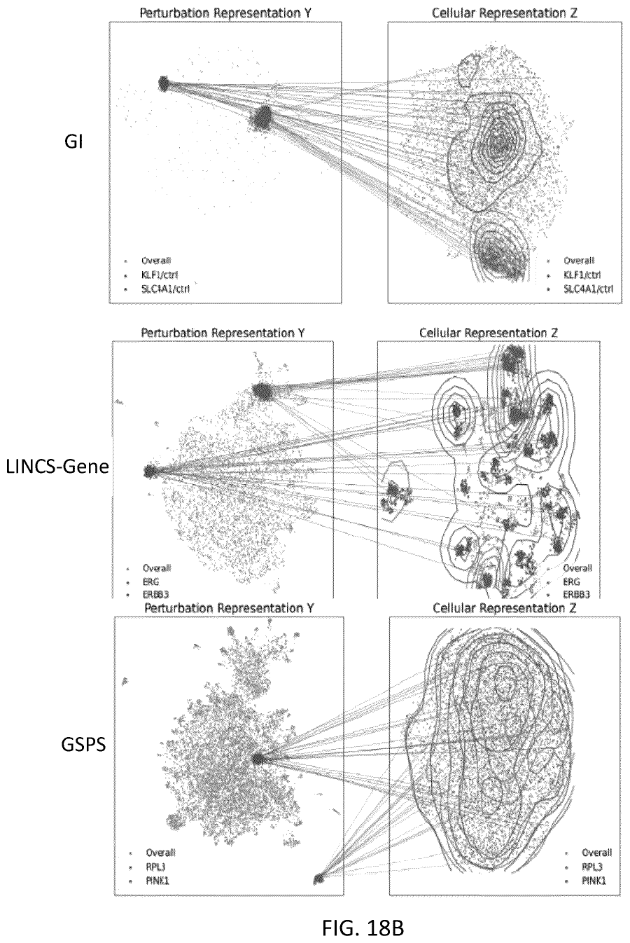

Some implementations utilized the ELBO loss and trained GenotypeVAE with a learning rate of 10-4 for 300 epochs. During the training, some implementations considered a probability of 0.5 for an iteration with a batch of genes to be genetic perturbations with single target genes, and another probability of 0.5 for the iteration to be used with the next batch of genes to be genetic perturbations with double target genes. Some implementations then evaluated the GenotypeVAE latent spaces for the genetic perturbations of the GI, LINCS-Gene and GSPS datasets. Both GI and GSPS have integer count scRNA-seq samples, while LINCS-Gene has normally distributed microarray samples. Some implementations therefore obtained their cellular representations by training scVl (Lopez, R., J. Regier, M. B. Cole, M. I. Jordan, and N. Yosef (2018), Deep generative modeling for single-cell transcriptomics, Nature methods, 15(12), 1053-1058) on the GI and GSPS data, as well as VAE on the LINCS. FIG. 18A shows the UMAP plots of perturbation representations and cellular representations for the two selected genetic perturbations in each of the three datasets. Both pairs of (KLF1/ctrl, SLC4A1/ctrl) in the GI data and (ERG, ERBB3) in the LINCS-Gene data have very different perturbation representations and cell state distributions. The perturbation representations of the pair of (RPL3, PINK1) in the GSPS data show distinctive distributions, while the difference between their cellular representations is less obvious. The perturbation representations have meaningful mappings to the cellular representations for the GI and LINCS-Gene datasets. The smaller difference in the cellular representation for the GSPS data might be due to its high batch effects (Replogle, J. M., et al. (2021), Mapping information-rich genotype-phenotype landscapes with genome-scale perturb-seq, bioRxiv).

Some implementations trained PerturbNet on the three datasets to recover the cellular representations from the perturbation representations. These implementations first partitioned the genetic perturbations to observed and unseen perturbations for each dataset. These implementations then used the cells with observed perturbations to train scVl on the GI and GSPS subset, as well as to train VAE on the LINCS-Gene subset. These implementations then connected the GenotypeVAE latent space and each cell latent space by training a conditional invertible neural network (cINN) on the cells with observed perturbations. From these steps, Perturb-Net was constructed for the three datasets. These implementations then utilized PerturbNet to reconstruct the cell latent values for the three pairs of genetic perturbations in FIG. 18B. The mappings between the perturbation representation and the cellular representation of the three perturbation pairs were precisely modeled by PerturbNet. More specifically, FIG. 18B shows example UMAP plots of perturbation representations and cellular representations as well as reconstructed cellular representations of three pairs of genetic perturbations in the GI, LINCS-Gene and GSPS datasets

PerturbNet Predicts Single-Cell Response to Genetic Perturbations

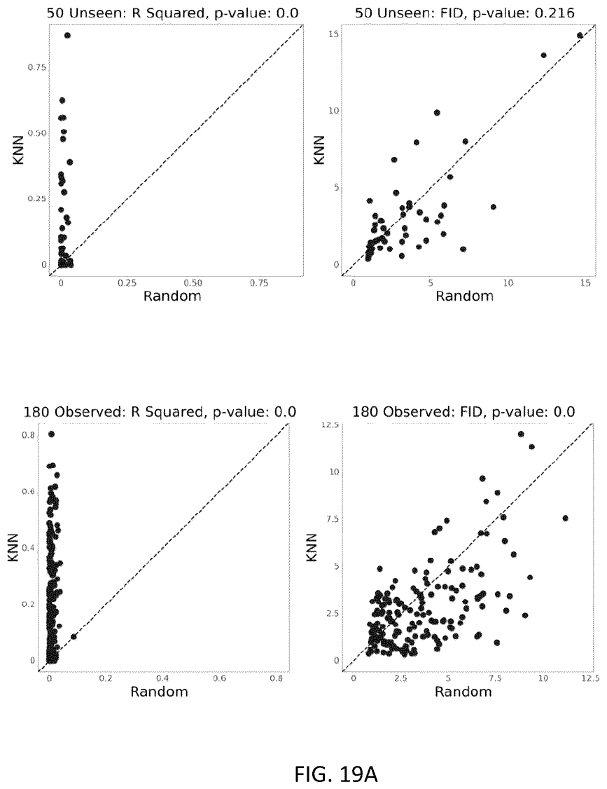

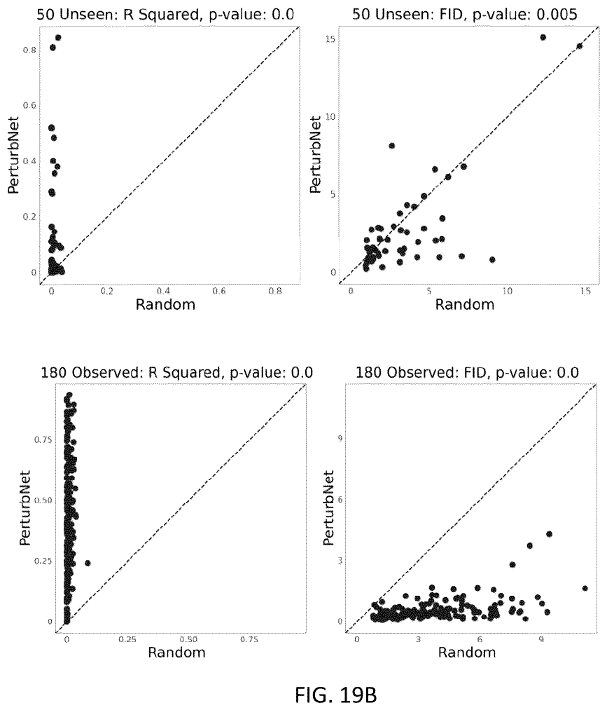

Some implementations predicted single-cell responses to each genetic perturbation using the baseline KNN, random models and PerturbNet for the three datasets. FIGS. 19A-19B show the performance of predicted cell samples evaluated with R squared and FID metrics of KNN and PerturbNet over random on the GI data. Both KNN and PerturbNet significantly outperform the random model for the 180 observed perturbations. The two models are also significantly better than random for unseen perturbations in R squared, but the KNN model does not have significantly lower FID than the random model for the 50 unseen perturbations.

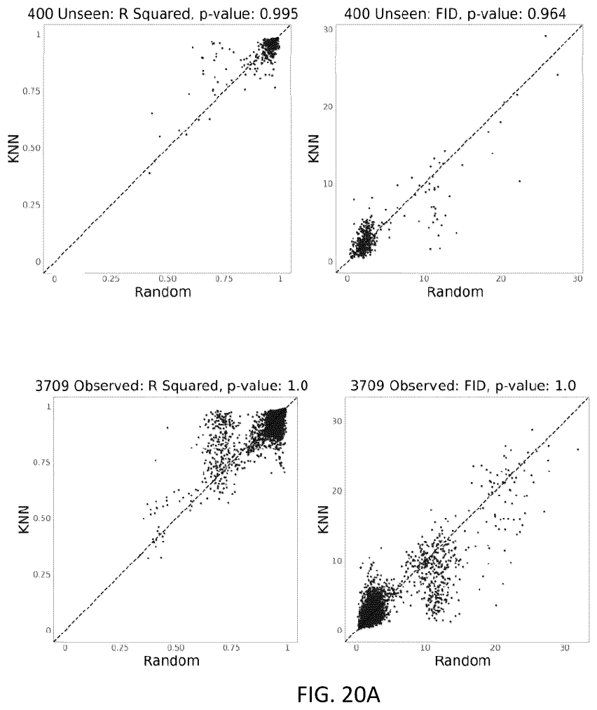

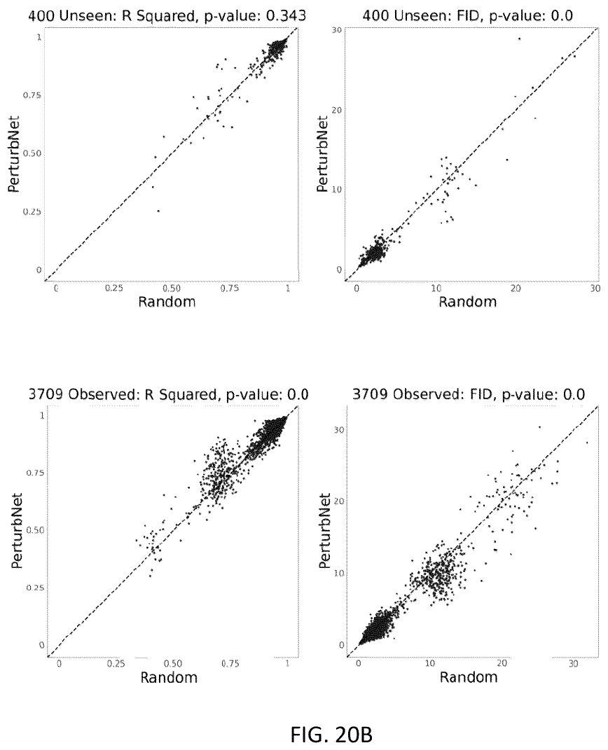

FIGS. 20A-20B show an example of the prediction performance of KNN and PerturbNet over random for the LINCS-Gene data. Although the KNN model does not outperform the random model for either unseen or observed perturbations, PerturbNet has significantly lower FID than the random model for both unseen and observed perturbations, and also has higher R squared for the observed perturbations. The random model shows very high R squared values (around 0.75) for the LINCS-Gene data, possibly because most genetic perturbations of the LINCS-Gene have small to medium perturbation effects. Some embodiments expect that, if focus is set on the subset of perturbations that have some detectable effect, the random model will be much less accurate and the PerturbNet will outperform it.

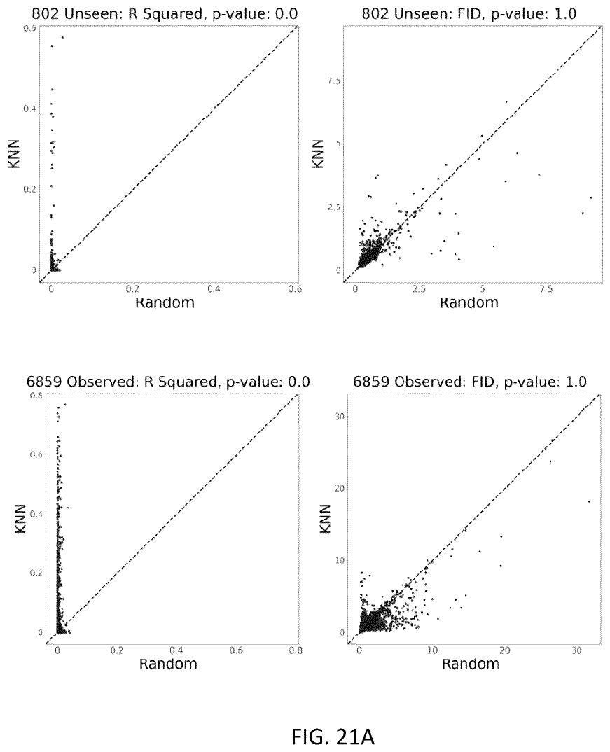

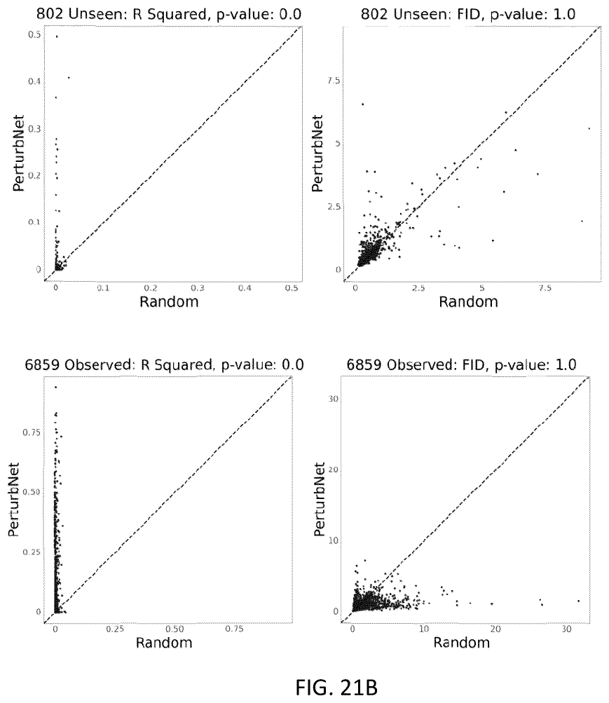

Some implementations evaluated the three predictive models on the Genetic Sample Processing System (GSPS) data (Replogle, J. M., et al. (2021), Mapping information-rich genotype-phenotype landscapes with genome-scale perturb-seq, bioRxiv), with a large number of target genes and a substantial proportion of perturbations with very few cells. Some embodiments filtered out the genetic perturbations with fewer than or equal to 100 cells before evaluating the KNN and random models. FIGS. 21A-21B show the performance of the three models on the 802 unseen and 6859 observed genetic perturbations with more than 100 cells of the GSPS data. Both KNN and PerturbNet have significantly higher R squared than random for either unseen or observed perturbations. However, neither shows better FID than random, possibly due to batch effects in this large-scale scRNA-seq data (Replogle, J. M., et al. (2021), Mapping information-rich genotype-phenotype landscapes with genome-scale perturb-seq, bioRxiv).

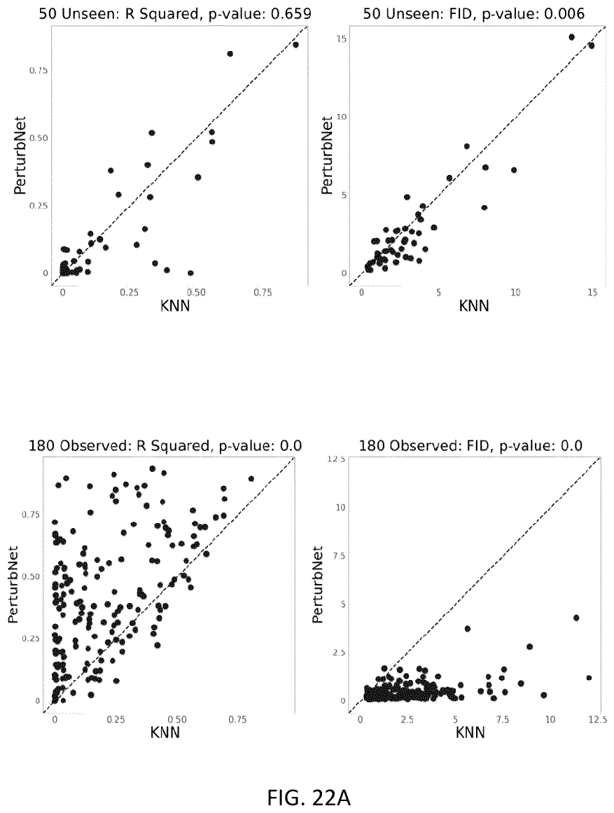

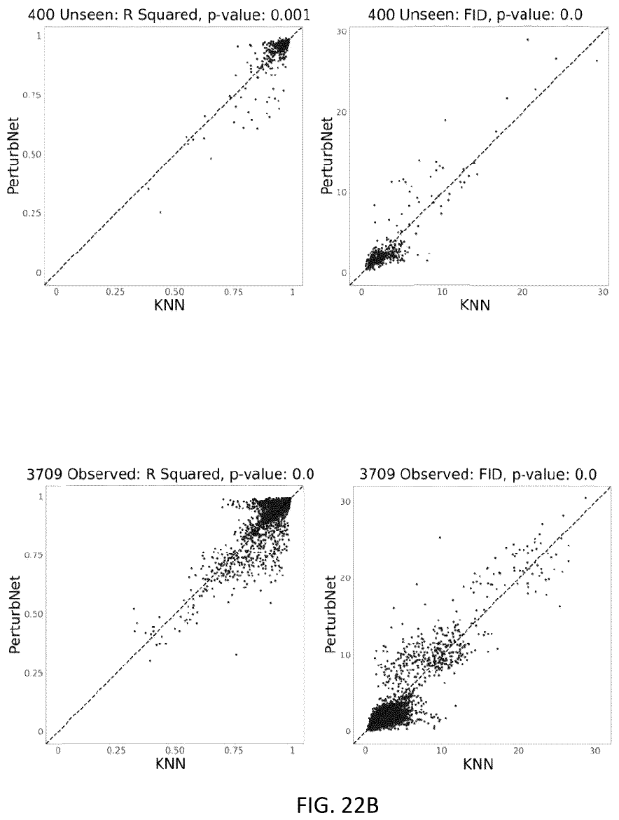

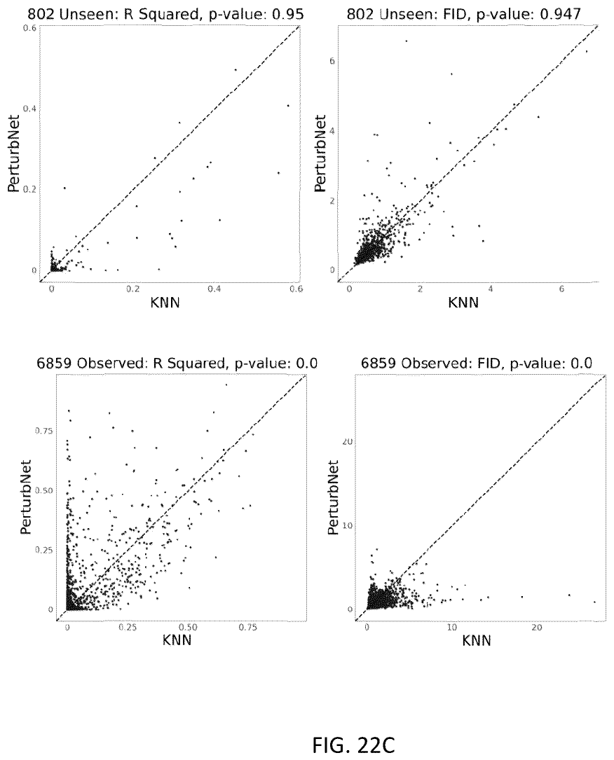

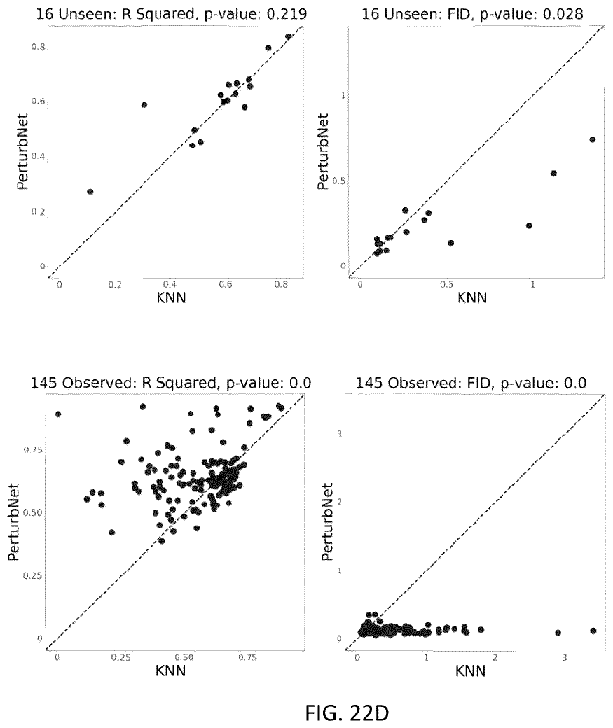

Some implementations also compared the performance between KNN and PerturbNet for the three datasets of the GI, LINCS-Gene and GSPS data (FIGS. 22A-22D). PerturbNet shows better predictions than KNN for the observed perturbations of the three datasets. PerturbNet also gives significantly better R squared and FID for unseen perturbations of the LINCS-Gene data, and better FID for unseen perturbations of the GI data. However, PerturbNet does not have a significant advantage over KNN for the unseen perturbations of the GSPS data, nor does it have better R squared for the unseen perturbations of the GI data. The limited number of 180 observed genetic perturbations learned in the GI data might not extrapolate well to the 50 unseen perturbations, and the batch effects in the GSPS data might have a negative impact on the predictions and evaluations of PerturbNet.

Fine-Tuned GenotypeVAE Improves Perturb-Net Performance for Genetic Perturbations

Some embodiments fine-tuned GenotypeVAE using the LINCS-Gene data following similar steps of the chemical variational autoencoder fine-tuning algorithm in Algorithm 3. Some embodiments first implemented the KNN algorithm on the perturbation representations of the genetic perturbations of the LINCS-Gene. Some implementations selected the five nearest neighbors of each perturbation, calculated the pairwise Wasserstein-2 (W2) distances between their cellular representations and set the distances between non-neighbors as 0's. Some implementations then averaged the distance matrix and its transposed matrix, calculated the exponential of their opposite values, and normalized to a unit sum for each row to obtain an adjacency matrix with each entry as a transition probability. Some implementations calculated the Laplacian L from the adjacency matrix, and used it as the graph to fine-tune GenotypeVAE. Some embodiments alternated the GenotypeVAE training with a batch of genetic perturbations from the large GO annotation dataset using the evidence lower bound (ELBO) loss and another batch of genetic perturbations from the LINCS-Gene data using the loss as follows:

Loss_{ϕ,θ}^{λ}=−ELBO(ϕ,θ)+λtrace(y^{T}L^{g}y)

where ϕ,θ were the parameters of the encoder and decoder of GenotypeVAE, and L_{g} and y were the Laplacian matrix and perturbation representations of the genetic perturbations in the batch.

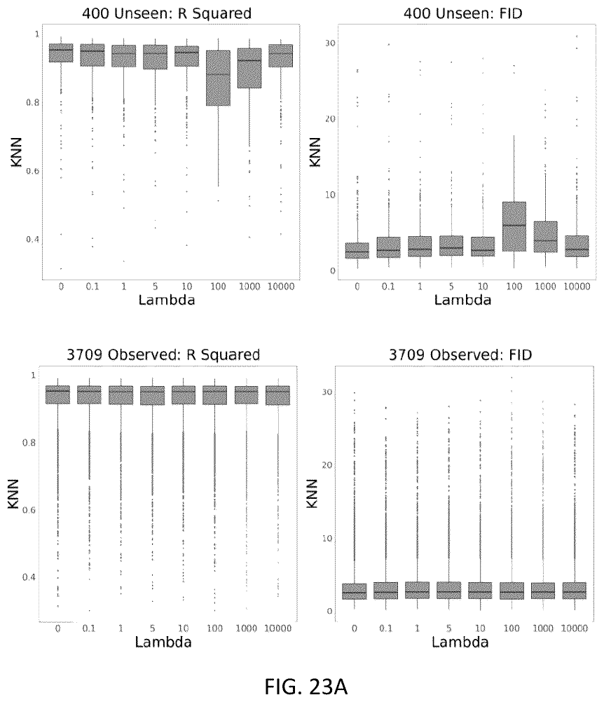

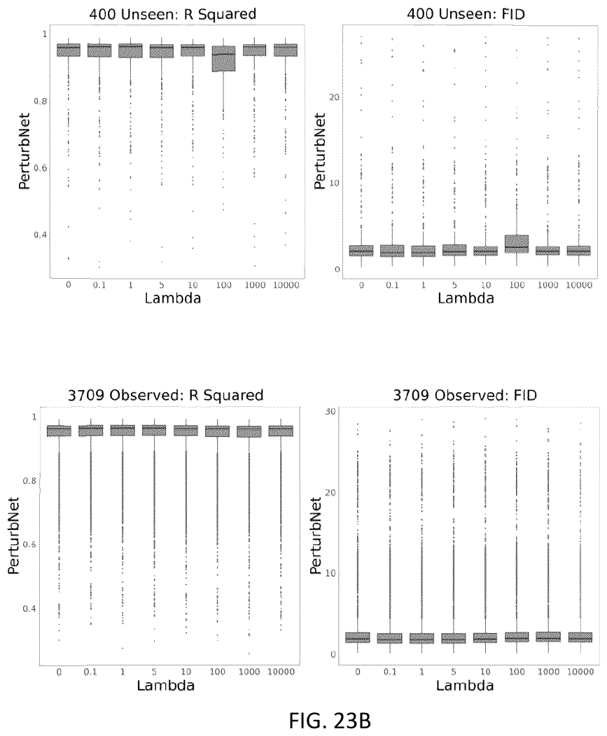

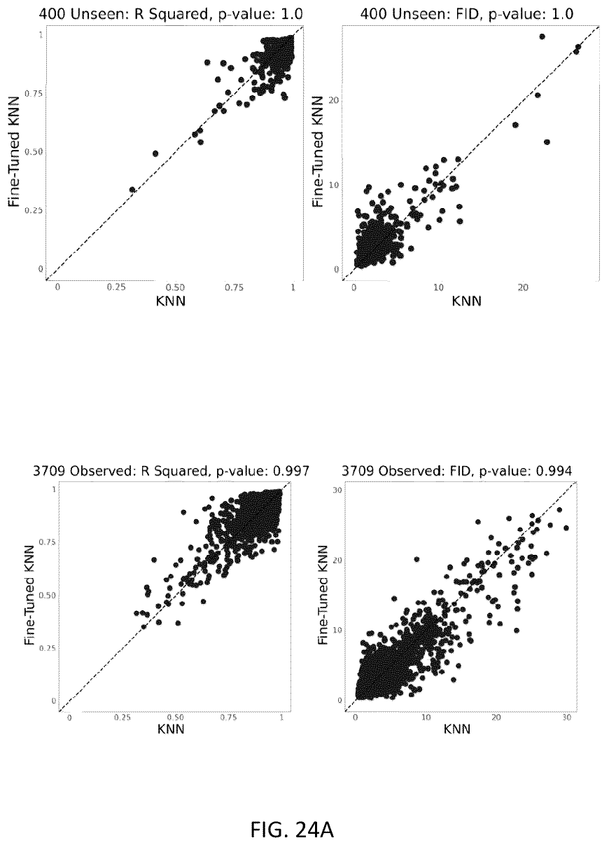

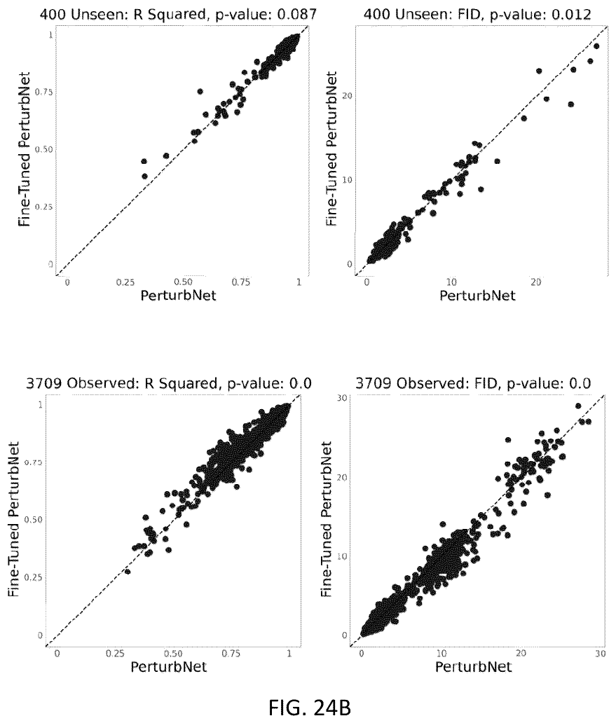

FIGS. 23A-23B show the R squared and FID of KNN and PerturbNet trained with fine-tuned GenotypeVAE (λ=0.1, 1,5, 10, 100, 1000, 10000). By comparing the evaluation metrics obtained from fine-tuned KNN and PerturbNet with different λ values, some embodiments determined that λ=1 was the optimal hyperparameter. FIGS. 24A-24B show the scatter plots of R squared and FID of KNN and PerturbNet with fine-tuned GenotypeVAE of λ=1 over those with non-fine-tuned GenotypeVAE. Fine-tuning GenotypeVAE significantly improves the performance of PerturbNet, especially for observed perturbations. Somewhat surprisingly, the fine-tuning algorithm improves only the PerturbNet, but does not significantly improve the performance of the KNN model.

PerturbNet Models Latent Representations of Protein Perturbations

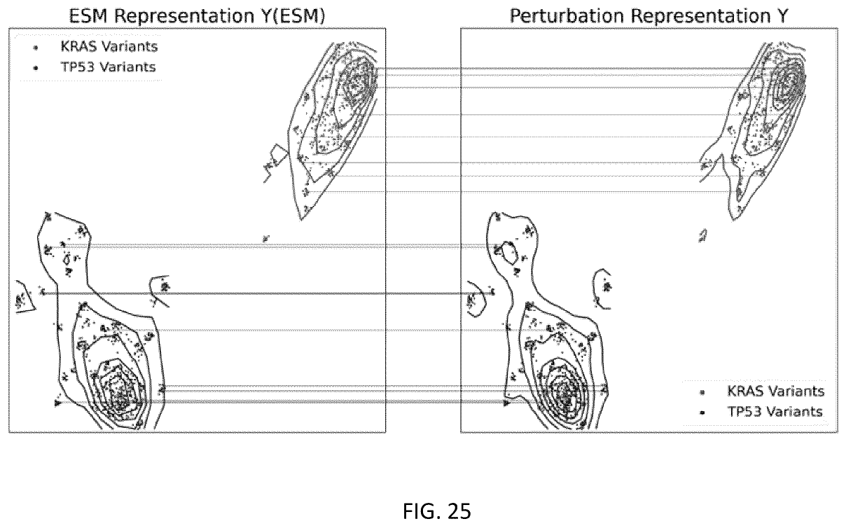

Some implementations evaluated the performance of PerturbNet for predicting single-cell responses to protein-coding sequence variants. To do this, some implementations used a Perturb-seq dataset of TP53 and KRAS variants introduced into the A549 cancer cell line (Ursu, O., et al. (2020), Massively parallel phenotyping of variant impact in cancer with perturb-seq reveals a shift in the spectrum of cell states induced by somatic mutations, bioRxiv). Some implementations preprocessed the detected CRISPR guide RNA sequences to obtain a single, complete protein sequence for each of the perturbations. Some implementations then used the pre-trained ESM transformer model to obtain deterministic representations of the protein perturbations. Some implementations experimented with the ESM representations by adding a small amount of Gaussian noise. Some implementations found that a noise term sampled from [symbol] (0,0.001I) effectively preserves the overall distribution of ESM representations (see FIG. 25).

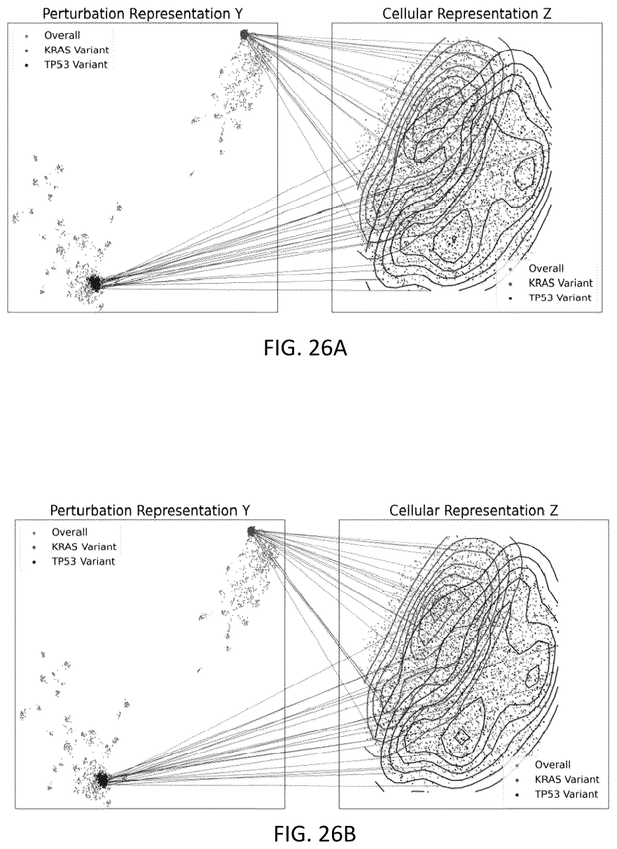

Some implementations trained scVl on the whole Ursu data and obtained the cellular representations of cells treated by a KRAS variant and a TP53 variant to visualize their mappings from perturbation representations to cellular representations (FIG. 26A). Both the perturbation representations and cellular representations have distinct distributions for the two variants. Some implementations also partitioned the perturbations of coding variants into 1208 observed and 130 unseen perturbations. Some implementations trained scVl on the cells with the observed perturbations and constructed the cINN translations of the Perturb-Net using the observed perturbation representation and cellular representation pairs. Some implementations utilized PerturbNet to reconstruct cellular representations from the perturbation representations (FIG. 26B). The perturbation-cellular representation mappings of the two variants were accurately restored.

PerturbNet Predicts Single-Cell Responses to Coding Sequence Mutations

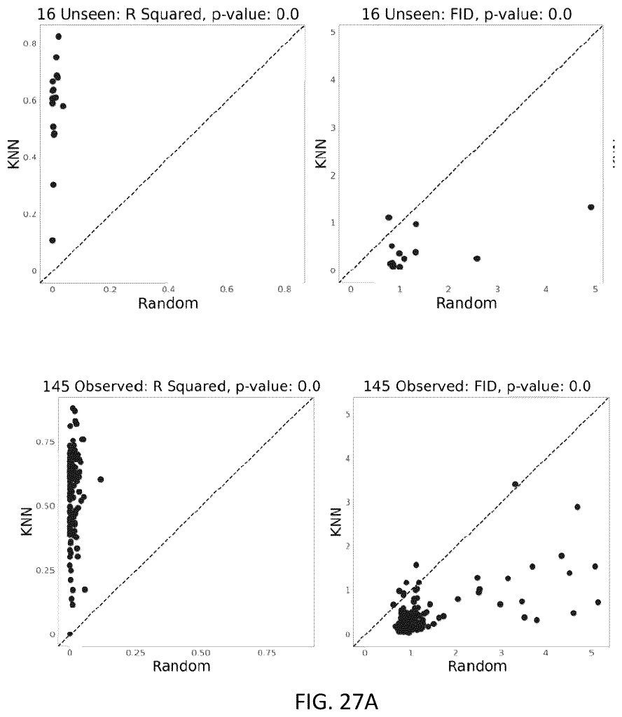

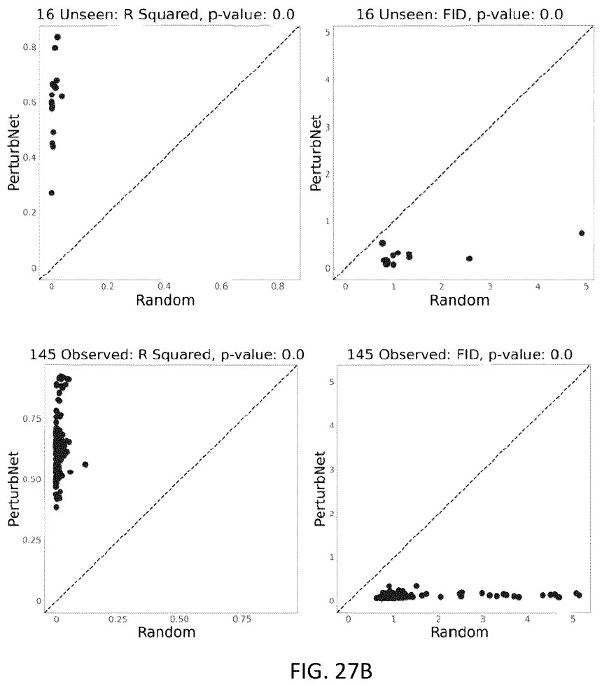

Some implementations employed KNN and PerturbNet to predict the single-cell responses to protein perturbations in the Ursu data. Some implementations found a large variability for the number of cells per perturbation and small numbers of cells for a substantial proportion of variants. Some implementations filtered the variants to those with more than 400 cells for the baseline KNN and random models. FIGS. 27A-27B show example representations of the R squared and FID metrics of KNN and PerturbNet over the random model for both filtered unseen and filtered observed perturbations. Both KNN and PerturbNet have significantly better metric values than the random model for either unseen or observed perturbations.

Some implementations also compared the performance between KNN and PerturbNet for the Ursu data (see, e.g., FIG. 22D). PerturbNet shows better predictions than KNN for the observed perturbations, and has better FID than KNN for the unseen perturbations. However, PerturbNet does not perform better than KNN for the unseen perturbations in R squared, possibly due to their limited number (16).

### DISCUSSION

Some implementations extend PerturbNet to predict single-cell responses to genetic perturbations. Some implementations consider two types of genetic perturbations from CRISPR gene editing experiments, including one with a set of target genes, and another with an edited coding sequence. Some implementations develop the GenotypeVAE model to encode the first kind of genetic perturbations with GO annotation features for the target genes. Some implementations call the second type of genetic perturbations “protein perturbations,” and employ a state-of-the-art transformer model (Meier, J., R. Rao, R. Verkuil, J. Liu, T. Sercu, and A. Rives (2021), Language models enable zero-shot prediction of the effects of mutations on protein function, Advances in Neural Information Processing Systems, 34) to encode variant sequences. Some implementations demonstrate that both KNN and PerturbNet predict the single-cell responses to genetic perturbations. Some implementations also fine-tune the GenotypeVAE using the LINCS-Gene data and improve the performance of PerturbNet.

A limitation of the study is that the framework primarily focuses on predicting single-cell responses to genetic perturbations in Perturb-seq data, and does not generalize to other single-cell data. With new advances in generalizability between Perturb-seq data and general single-cell data, future research may infer single-cell perturbation responses for single-cell data such as the Tabula Muris compendium (Consortium, T. M., et al. (2018), Single-cell transcriptomics of 20 mouse organs creates a tabula muris, Nature. 562(7727), 367).

As representation learning techniques are rapidly evolving, future improvements of genetic perturbations can be utilized for PerturbNet. For example, one work (Van Den Oord, A., O. Vinyals, et al. (2017), Neural discrete representation learning, Advances in neural information processing systems, 30) introduced the VQ-VAE framework that learns discrete latent representations with very strong semantic meanings. In addition, VQ-VAE can model sequences with long term dependencies such as those in the coding variants of protein perturbations. The GenotypeVAE model could also be extended to model the hierarchical structure of GO terms (Ma, J., M. K. Yu, S. Fong, K. Ono, E. Sage, B. Demchak, R. Sharan, and T. Ideker (2018), Using deep learning to model the hierarchical structure and function of a cell, Nature methods, 290-298), rather than simply using a one-hot vector as in some implementations of the present disclosure.

Datasets

Some implementations obtained the GO annotation dataset for proteins of Homo sapiens from GO Consortium at http://geneontology.orq/docs/guide-go-evidence-codes. Some implementations removed the annotations of three sources without sufficient information: inferred from electronic annotation (IEA), no biological data available (ND) and non-traceable author statement (NAS). The filtered dataset had possible annotations for 18,832 genes.

Some implementations obtained the GI data on GEO accession ID GSE133344 (Norman, T. M., M. A. Horlbeck, J. M. Replogle, Y. G. Alex, A. Xu, M. Jost, L. A. Gilbert, and J. S. Weissman (2019), Exploring genetic interaction manifolds constructed from rich single-cell phenotypes, Science, 365(6455), 786-793). Each cell was perturbed with 0, 1 or 2 target genes. Some implementations processed the GI data using SCANPY (Wolf, F. A., P. Angerer, and F. J. Theis (2018), Scanpy: large-scale single-cell gene expression data analysis, Genome biology, 19(1), 15) with 109,738 cells and 2279 genes. The processed GI data contained 236 unique genetic perturbations for 105 target genes and 11,726 cells were unperturbed. There were 230 out of 236 genetic perturbations that could be mapped to the GO annotation dataset. Some implementations randomly selected 50 genetic perturbations as unseen and the other 180 perturbations as observed.

Some implementations obtained the LINCS dataset (Subramanian, A., et al. (2017), A next generation connectivity map: L1000 platform and the first 1,000,000 profiles, Cell, 171 (6), 1437-1452) from GEO accession ID GSE92742. The LINCS data had been processed with 1,319,138 cells and 978 landmark genes. The LINCS-Gene subset of the LINCS data contained 442,684 cells treated by 4371 genetic perturbations with single target genes. The 4109 out of 4371 genetic perturbations could be mapped to the GO annotation dataset, and some implementations randomly selected 400 genetic perturbations as unseen perturbations and the other 3709 as observed perturbations.

Some implementations used SCANPY to preprocess the GSPS data (Replogle, J. M., et al. (2021), Mapping information-rich genotype-phenotype landscapes with genome-scale perturb-seq, bioRxiv) and to select the top 2000 highly-variable genes with respect to the batches of ‘gemgroups’. The GSPS dataset contained 1,989,373 cells treated by 9867 genetic perturbations with single target genes. There were 9499 genetic perturbations that can be mapped to the GO annotation library. Some implementations randomly selected 1000 genetic perturbations as unseen perturbations and the other 8499 as observed perturbations. There were 802 unseen and 6859 observed perturbations, each with more than 100 cells.

Some implementations obtained the Ursu data from GEO accession ID GSE161824, and filtered the raw data according to the processed datasets and concatenated the two datasets with KRAS variants and TP53 variants, using their common genes. Some implementations preprocessed the concatenated data using SCANPY, containing 164,931 cells and 1629 genes. Some implementations also collected the variants from the modifications on the original KRAS and TP53 protein sequences. Some implementations obtained 596 KRAS sequences and 742 TP53 protein sequences, and randomly selected 60 KRAS and 70 TP53 variants as unseen perturbations. There were 16 unseen and 145 observed variants with more than 400 cells.

Perturbation Design and Biological Discovery with PerturbNet—Introduction

The discussion above shows that PerturbNet can successfully predict the effects of unseen perturbations. The following discussion uses the predictive modeling capabilities of PerturbNet to (1) design perturbations with desired effects on cell state and (2) discover the features of perturbations that predict their effects.

To summarize the above discussion: The PerturbNet framework has been shown to effectively model cellular responses from perturbations. It first learns perturbation and cellular representations independently through two powerful VAE-based models, avoiding potential interference of unbalanced joint distribution between perturbation and cell state of the method by one previous work (Lotfollahi, M., M. Naghipourfar, F. J. Theis, and F. A. Wolf (2020), Conditional out-of-distribution generation for unpaired data using transfer vae, Bioinformatics, 36(Supplement 2), i610-i617). Then, conditional invertible neural networks (cINN) connect the perturbation representation and cellular representation from individual cells. Therefore, the PerturbNet framework comprises a flexible multi-stage modeling process to learn representations and their relationships. The learned relationships among representations can further predict out-of-distribution or counterfactual cellular representations and cellular responses under various perturbations.

The flexibility of PerturbNet to predict out-of-distribution cellular responses provides pragmatic guidance on designing perturbations. For instance, characterizing counterfactual profiles of a group of diseased cells to several drug treatments may bring a potential cure for the disease or more understanding about an optimal drug to achieve the desired responses. Additionally, CRISPR genetic perturbations usually focus on a limited set of target genes due to the time-consuming and expensive experiments with potential side effects. The counterfactual profiles predicted by PerturbNet enhance the understanding of a desired genetic perturbation to shift the cell state of a group of cells, providing potential therapeutics for genetic diseases such as HIV. Thus, designing perturbations efficiently advances biomedical development, and can be guided from counterfactual cell responses predicted by PerturbNet.

These counterfactual responses not only enable perturbation design, but also reveal key components or functions in a chemical or genetic perturbation that influence the cell state. Estimating counterfactual responses is a fundamental problem and has numerous implications for understanding the heterogeneous effects of drugs (Shalit, U., F. D. Johansson, and D. Sontag (2017), Estimating individual treatment effect: generalization bounds and algorithms, in International Conference on Machine Learning, pp. 3076-3085, PMLR; Alaa, A. M., and M. van der Schaar (2017), Bayesian inference of individualized treat-ment effects using multi-task gaussian processes, Advances in Neural Information Processing Systems, 30). In these studies, some measurable quantitative trait serves as the potential outcome variable, and the heterogeneous effects are quantified and interpreted by individual treatment effect (ITE), which is the difference between the mean outcomes of a perturbation of interest and a baseline perturbation. High-dimensional single-cell responses, however, possess complex cellular information, and ITE does not uncover the underlying perturbation effects on the cell state. As a single-cell expression profile defines its cell state, understanding single-cell profiles of perturbations directly enables interpreting perturbation effects. Furthermore, PerturbNet's ability to predict cell states from perturbation features (atoms or gene ontology terms) provides an opportunity to hone in on mechanisms by implicating specific perturbation features. Recent developments in explainable artificial intelligence (Gilpin, L. H., D. Bau, B. Z. Yuan, A. Bajwa, M. Specter, and L. Kagal (2018), Explaining explanations: An overview of interpretability of machine learning, in 2018 IEEE 5th International Conference on data science and advanced analytics (DSAA), pp. 80-89, IEEE) have improved the transparency of model details and reasoning. Many model interpretability methods for neural networks can attribute classification decisions to certain key features of the input data (Shrikumar, A., P. Greenside, A. Shcherbina, and A. Kundaje (2016), Not just a black box: Learning important features through propagating activation differences, arXiv preprint arXiv:1605.01713; Shrikumar, A., P. Greenside, and A. Kundaje (2017), Learning important features through propagating activation differences, in International conference on machine learning, pp. 3145-3153, PMLR; Selvaraju, R. R., M. Cogswell, A. Das, R. Vedantam, D. Parikh, and D. Batra (2017), Grad-cam: Visual explanations from deep networks via gradient-based localization, in Proceedings of the IEEE international conference on computer vision, pp. 618-626; Mudrakarta, P. K., A. Taly, M. Sundararajan, and K. Dhamdhere (2018), Did the model understand the question?, arXiv preprint arXiv:1805.05492). These model interpretability methods usually involve interpreting the features of an input tensor compared to a baseline tensor in terms of their effects on the outcome of a neural network classification model (Kokhlikyan, N., et al. (2020), Captum: A unified and generic model interpretability library for pytorch, arXiv preprint arXiv:2009.07896). In the context of the present work, these interpretability methods enable determination of which input features make cells more likely to be in a particular cell state.

The following discussion uses the counterfactual prediction capability of PerturbNet to design optimal perturbations that achieve desired effects. Some implementations consider a group of real cells treated by some perturbation, and aim to learn an alternative perturbation that can translate these cells to approximate a desired cell state. To achieve the cell state translation, some implementations utilize PerturbNet to extract the residual representation from pairs of perturbation and cellular representations, and to predict the counterfactual cellular representation under another perturbation. Some implementations propose two algorithms to design perturbations that optimally translate cells from the starting cell state to the desired cell state.

In addition, some implementations interpret the predictive model to implicate key perturbation features that influence cell state distributions. Some implementations employ the method of integrated gradients to determine, for each input feature, whether the presence of the feature increases or decreases the probability of cells being in a particular state. Some implementations also interpret the attributions of the optimal chemical perturbation in the optimal translations. Finally, some embodiments identify GO terms that contribute greatly to the formation of different cell state distributions between two genetic perturbations.

Methods—Optimal Perturbation Design

Consider a starting cell state with latent space values τ₁(Z), and a target cell state with latent space values τ₂(Z). It may be desirable to find a perturbation that changes the cells in the starting cell state to the target cell state. From PerturbNet trained with single-cell perturbation responses, some implementations obtain the encoded representations for m cells in the starting cell state with the latent values {z₁, . . . , zₘ}˜τ₁(Z). Each starting cell is originally treated with a perturbation. For simplicity, some implementations assume that these cells are treated with the same perturbation g₁. The target cell state can be represented by the latent values of n cells {zₘ₊₁, . . . , Zₘ₊ₙ}˜τ₂(Z). The optimal translation task thus aims to find an alternative perturbation g* for the starting cells to change their cell state to be close to τ₂(Z).

As PerturbNet translates perturbation representation Y and residual representation V to cellular representation Z, some implementations predict the counterfactual cell state under a new perturbation for each cell with two translation procedures. Denote cINN forward translation as f(·), B₁ as the perturbation matrix of the starting perturbation g₁ and the perturbation encoder as h(·). First, some implementations obtain residual values {v₁, . . . , vₘ} with the inverse translation function vᵢ=f⁻¹(zᵢ|yᵢ) with perturbation representation yᵢ=h(B₁). The translation function then gives each cell's counterfactual cellular representation z_{i,*}f(vᵢ|y*) under an alternative perturbation's representation value y*. Some embodiments therefore seek the translated counterfactual cell state {z_{1,*}, . . . , z_{m,*}}˜τ_{*}(Z) to have a similar distribution to {zₘ₊₁, . . . , zₘ₊ₙ}˜τ₂(Z).

Continuous Optimal Translation

Some implementations devise a method to design a perturbation representation y* that shifts the cells in the starting cell state to approximate the target cell state. To quantify the difference between the cell state distributions, some implementations use Wasserstein distance, which has been used to quantify cell populations' distance (Schiebinger, G., et al. (2019), Optimal-transport analysis of single-cell gene expression identifies developmental trajectories in reprogramming, Cell, 176(4), 928-943; Crowley, L., et al. (2020), A single-cell atlas of the mouse and human prostate reveals heterogeneity and conservation of epithelial progenitors, Elife, 9, e59,465; Demetci, P., R. Santorella, B. Sandstede, W. S. Noble, and R. Singh (2020), Gromov-wasserstein optimal transport to align single-cell multi-omics data, BioRxiv). Some implementations use Wasserstein-2 (W2) distance (Vaserstein, L. N. (1969), Markov processes over denumerable products of spaces, describing large systems of automata, Problemy Peredachi Informatsii, 5(3), 64-72), which is also known as Fréchet distance, to quantify the dissimilarity between the cell state distributions of τ₂(Z) and τ_{*}(Z). The W2 distance is defined as

$$\\ d \{ {τ}_{2} ( Z ) , {τ}_{*} ( Z ) \} = {\{ {inf}_{γ ∈ \prod ( {τ}_{2} , {τ}_{*} )} {𝔼}_{( {Z}_{2} , {Z}_{*} ) \sim γ} {‖ {Z}_{2} - {Z}_{*} ‖}^{2} \}}^{1 / 2}$$

where Πτ₂,τ_{*} is the set of all joint distributions γ(Z₂,Z_{*}) whose marginal distributions are τ₂(Z) and τ_{*}(Z), respectively.

Evaluating the W2 distance is extremely difficult for general distributions. To simplify the calculations of the W2 distance, some implementations assume that latent spaces follow multivariate Gaussian distributions (Dowson, D., and B. Landau (1982), The fréchet distance between multivariate normal distributions, Journal of multivariate analysis, 12(3), 450-455; Heusel, M., H. Ramsauer, T. Unterthiner, B. Nessler, and S. Hochreiter (2017), Gans trained by a two time-scale update rule converge to a local nash equilibrium, in Advances in neural information processing systems, pp. 6626-6637), which is also commonly assumed in calculating Fréchet inception distance (FID) in image data (Heusel, M., H. Ramsauer, T. Unterthiner, B. Nessler, and S. Hochreiter (2017), Gans trained by a two time-scale update rule converge to a local nash equilibrium, in Advances in neural information processing systems, pp. 6626-6637). Assuming that the latent space τᵢ(Z) has a multivariate Gaussian distribution [symbol] (μᵢ,Σᵢ) for i∈{2,*}, the squared W2 distance has a closed form of the following Equation (2):

d²{τ₂(Z),τ_{*}(Z)}=∥μ₂−μ_{*}∥₂²+trace{Σ₂+Σ_{*}−2(Σ₂Σ_{*})^{1/2}}

Therefore, some embodiments evaluate the squared W2 distance between the translated counterfactual cell state and the target cell state as d²[{zₘ₊ⱼ}ⱼ₌₁ⁿ], {z_{i,*}}ᵢ₌₁^{m}. The problem of designing a desired perturbation is then to find the optimal yₒₚₜ* that minimizes the squared W2 distance:

$$\\ {y}_{opt}^{*} = {arg min}_{{y}^{*}} {d}^{2} [ {\{ {z}_{m + j} \}}_{j = 1}^{n} , {\{ {z}_{i , *} \}}_{i = 1}^{m} ]$$

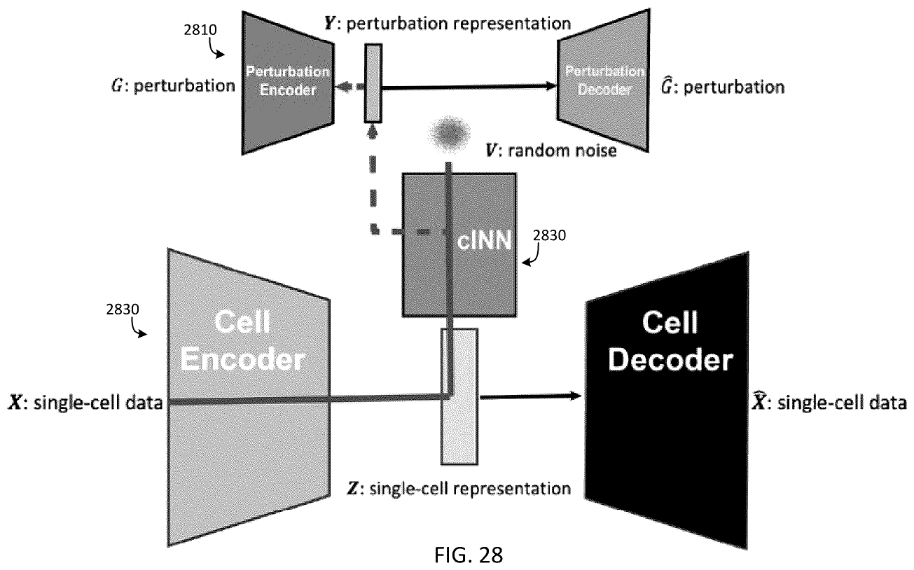

Some embodiments further infer the optimal perturbation from representation yₒₚₜ^{*}. FIG. 28 summarizes the procedure to design the optimal perturbation using PerturbNet, including first network 2810, second network 2820, and third network 2830.

Based on the objective above, some embodiments propose what may be referred to as “continuous optimal translation.” Some implementations first initialize a value for yₒₚₜ* from the standard multivariate Gaussian distribution and then some embodiments perform stochastic gradient descent with momentum (Kingma, D. P., and J. Ba (2014), Adam: A method for stochastic optimization, arXiv preprint arXiv:1412.6980) to minimize the squared W2 loss over yₒₚₜ^{*}. One implementation detail concerns the calculation of the W2 distance. The distance formula includes the term (Σ₂Σ_{*})^{1/2}, which is difficult to calculate and can become ill-conditioned or approximately singular. Some embodiments thus rewrite the term as

C_{2,*}=Σ₂^{1/2}(Σ₂^{1/2}Σ_{*}Σ₂^{1/2})^{1/2}Σ₂^{-1/2}

which allows to replace the difficult term with C_{2,*}² as C_{2,*}²=Σ₂Σ_{*}.

Some implementations use the Adam optimizer to perform stochastic gradient descent with momentum. For the matrix square root terms in C_{2,*}, Σ₂^{1/2} keeps a fixed value during training, and Σ₂^{1/2}Σ_{*} Σ₂^{1/2} is much more likely than the original term Σ₂Σ_{*} to be symmetric positive semi-definite and have a square root matrix. After the continuous optimization, some implementations obtain an optimal perturbation representation yₒₚₜ* that represents a potential perturbation that achieves the desired shift in cell state distribution.

Discrete Optimal Translation

The continuous optimal translation model can give an optimal perturbation representation yₒₚₜ* that translates the starting cells to have a similar cell state to τ₂(Z). If the real cells in the target cell state {zₘ₊₁, . . . , Zₘ₊ₙ}˜τ₂(Z) are treated by a perturbation g₂, some implementations compare it with the fitted optimal perturbation representation yₒₚₜ* to evaluate if the optimal perturbation representation can achieve the desired cell state shift like the perturbation g₂.

However, the chemical or genetic perturbation from the optimal perturbation representation of a continuous optimal translation is not immediately clear, as an inference model needs to be processed on the perturbation representation. Although it is possible to employ the perturbation generative model to generate chemical or genetic perturbations, doing so brings a host of additional challenges related to molecular structure optimization (Gömez-Bombarelli, R., et al. (2018), Automatic chemical design using a data-driven continuous representation of molecules, ACS central science, 4(2), 268-276), which is not the focus of this disclosure.

To design the optimal perturbation to achieve the desired cell state shift, some implementations propose another perturbation design strategy that uses discrete optimization. Rather than optimizing the squared W2 loss in the continuous space, the discrete optimal translation searches through a constrained set G of perturbations, and calculates the squared W2 distance d²[{zₘ₊ⱼ}ⱼ₌₁ⁿ,{z_{i,*}}ᵢ₌₁^{m}] for each perturbation g∈ [symbol] with y*=h(B_{g}). Then the optimal perturbation is selected as the one giving the smallest distance so that

$$\\ {ℊ}_{opt} = {{arg min d}_{g ∈ 𝒢}}^{2} [ {\{ {z}_{m + j} \}}_{j = 1}^{n} , {\{ {z}_{i , *} \}}_{i = 1}^{m} ]$$

This discrete optimal translation strategy gives both the optimal perturbation representation yₒₚₜ* to achieve the desired translation, and also the optimal perturbation gₒₚₜ. If the cells in the target latent space are treated by a certain perturbation, some embodiments can evaluate if the optimal perturbation gₒₚₜ matches the one for the target latent space.

Model Interpretation Using Integrated Gradients

As perturbation and cell state are connected in PerturbNet, some implementations can interpret how a perturbation changes the cell state distribution by predicting cellular representations using PerturbNet. Some implementations further interpret the effects of features and components of the perturbation with the state-of-the-art explainable artificial intelligence methods. Denote F(·) as a function taking input feature vector T=(T₁, . . . , Tₙ)^{T}∈ [symbol] ⁿ. to generate output in [0, 1]. Then its attribution is a vector A=(a₁, . . . , aₙ)^{T} and each value aᵢ is the contribution of Tᵢ to the prediction of F(T).

Previous attempts to interpret neural network models have focused on gradients (Baehrens, D., T. Schroeter, S. Harmeling, M. Kawanabe, K. Hansen, and K.-R. Müller (2010), How to explain individual classification decisions, The Journal of Machine Learning Research, 11, 1803-1831; Simonyan, K., A. Vedaldi, and A. Zisserman (2013), Deep inside convolutional networks: Visualising image classification models and saliency maps, arXiv preprint arXiv:1312.6034) and back-propagation (Shrikumar, A., P. Greenside, A. Shcherbina, and A. Kundaje (2016), Not just a black box: Learning important features through propagating activation differences, arXiv preprint arXiv:1605.01713; Shrikumar, A., P. Greenside, and A. Kundaje (2017), Learning important features through propagating activation differences, in International conference on machine learning, pp. 3145-3153, PMLR). Some embodiments use the method of integrated gradients (Sundararajan, M., A. Taly, and Q. Yan (2017), Axiomatic attribution for deep networks, in International conference on machine learning, pp. 3319-3328, PMLR), which has been applied to interpret deep learning models across a range of domains, including computational chemistry (McCloskey, K., A. Taly, F. Monti, M. P. Brenner, and L. J. Colwell (2019), Using attribution to decode binding mechanism in neural network models for chemistry, Proceedings of the National Academy of Sciences, 116(24), 11,624-11,629). The attribution score of the integrated gradients method for the ith dimension of input T is defined as

$$\\ {a}_{i} = ( {T}_{i} - {T}_{0 , i} ) {{\int }^{1}}_{α = 0} \frac{\partial F \{ {T}_{0} + α ( T - {T}_{0} ) \}}{\partial {T}_{i}} d α$$

where T₀=(T_{0,0}, . . . , T_{0,n})^{T} is a baseline input.

A prediction neural network model on cellular representation can be formulated from PerturbNet as Z=f(V|Y) and Y=h(B). The input T can be formulated as (V^{T}, Y^{T})^{T} or (V^{T},B^{T})^{T}. In addition, a classification neural network model on Z provides a classification score within [0, 1]. Some embodiments then find input features that increase the probability of generating cells in a particular cell state.

Experiments—Continuous Optimal Translation for Perturbation Representations

Some implementations performed continuous optimal translation on the chemical perturbations of the sci-Plex and LINCS-Drug datasets. For PerturbNet trained on sci-Plex without adjusting for cell state covariates, some implementations used the latent values of cells treated by S1172 as the starting latent space, and considered the target latent space as the latent values of each of the 158 observed drug treatments. For the LINCS-Drug dataset, some implementations also fixed a drug treatment as the starting perturbation and used each of the 200 selected observed drug treatments as the target perturbation. For each translation on the two datasets, some implementations trained the continuous optimization algorithm for 600 epochs to obtain the optimal perturbation representation. As the cells of the target latent space are also treated with a perturbation, some implementations also evaluated the translation using the perturbation representation of the target perturbation.

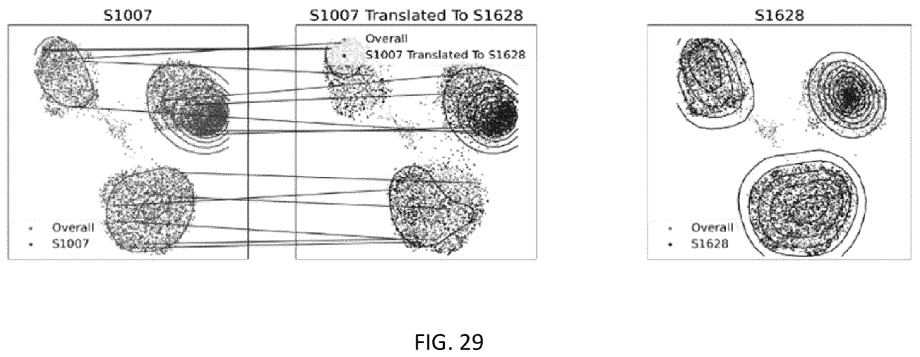

The target perturbation, however, does not necessarily translate the cells in the starting latent space to overlap with the target latent space. FIG. 29 shows a translation example to translate the cells treated by S1007 to a target latent space using the target perturbation S1628. As can be seen, the translated counterfactual latent values from S1007 to S1628 have a distinct distribution from the latent values of real cells treated by S1628. This distinction is due to the fact that the residual representation V of the real cells treated by S1007 is different from that of S1628, and therefore a translation of S1007 cells using S1628 potentially gives a different latent distribution. Thus, the target perturbation might not perform well in translating the starting latent space to approximate the target latent space, and might even enlarge the original distance between the two latent spaces.

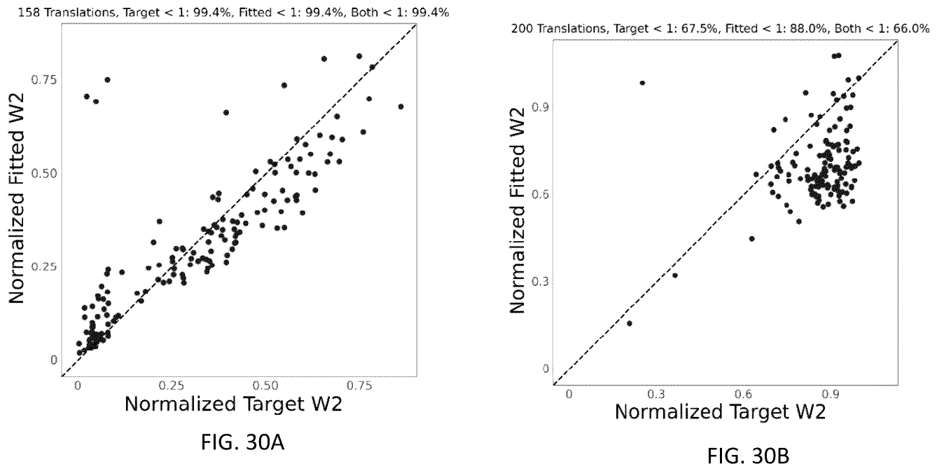

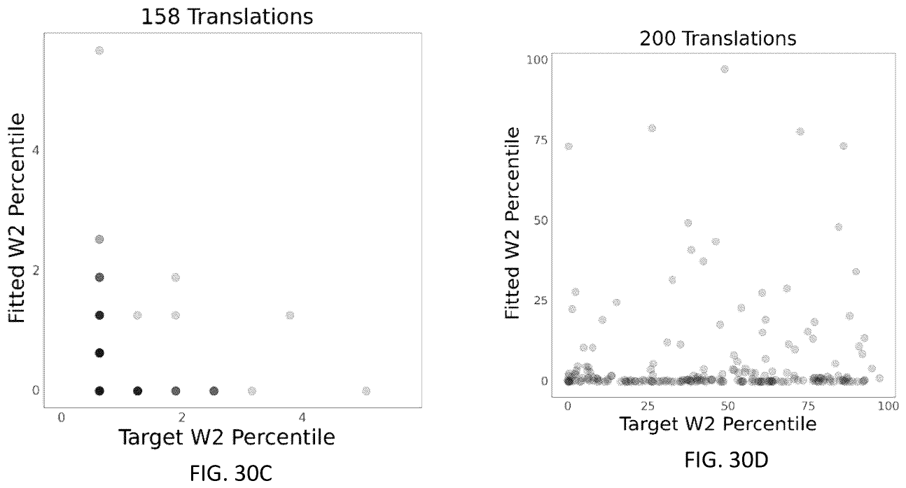

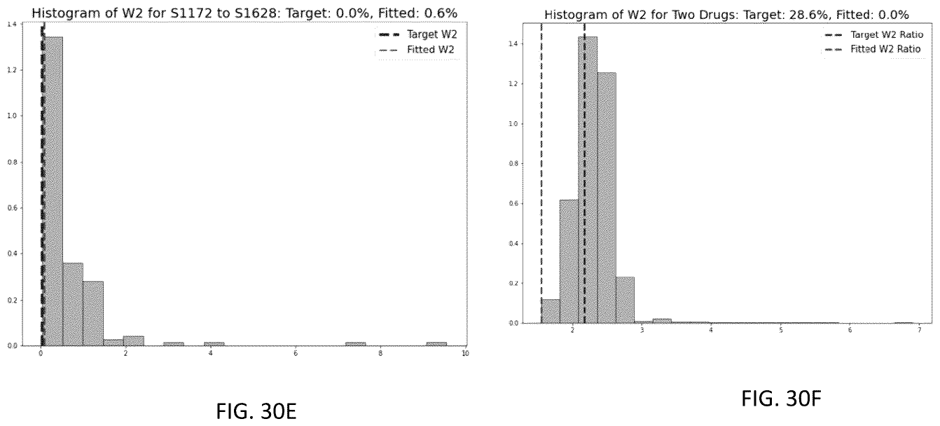

FIGS. 30A-30F show examples of the continuous optimal translations for the sci-Plex and LINCS-Drug datasets. For simplicity, some embodiments named squared W2 distance and W2 distance interchangeably, both corresponding to the form in Equation (2). Some embodiments calculated the W2 distances between the target latent space and the translated latent space, using either the fitted perturbation representation or the target perturbation representation. Some implementations then normalized the two distances by the original W2 distance between the starting latent space and target latent space, and called them normalized fitted W2 distance and normalized target W2 distances. A normalized W2 distance smaller than 1 means that the translation reduces the original distance between the two latent spaces. Some implementations show the scatter plots of the normalized fitted W2 and normalized target W2 for the sci-Plex (FIG. 30A) and LINCS-Drug (FIG. 30B) data. Some implementations found that 99.4% of the translations using either fitted or target perturbation effectively reduced the original latent distances for sci-Plex data. The fitted perturbation representation has an overall close performance to the target perturbation. For the LINCS-Drug data, the target perturbation can shrink the original latent distances in 67.5% of the 200 translations, while the fitted perturbation representations have a better performance to shrink the distance in 88% of the translations. For translations shrinking the original latent distance from both target representation and fitted representation, the fitted representation can sometimes perform better than the target perturbation (FIG. 30B). This means that the target perturbation is possibly not optimal to reduce the latent distances, and the continuous optimal translation algorithm provides a better perturbation representation to achieve the latent approximation.

Some implementations also plot the percentiles of the fitted W2 and target W2 in the distribution of W2 distances between the target latent space and the 2000 translated latent spaces across the 200 translations for the sci-Plex (FIG. 30C) and LINCS-Drug (FIG. 30D) data. Both the fitted and target perturbations tend to give W2 percentiles of 0's for the sci-Plex translations, while LINCS-Drug has small percentile values for fitted W2 but varying target W2 percentiles. The LINCS-Drug dataset has many more perturbations than the sci-Plex and most of the perturbations have small numbers of cells. As a result, the starting and target latent spaces of LINCS-Drug might possess an overall larger difference in their residual representations, and the translation using the target perturbation possibly does not perfectly approximate the target latent space.

To evaluate how well the fitted perturbation representation translates the starting latent space to approximate the target latent space in a translation, some embodiments computed the W2 distances between the target latent space and a translated latent space using each of the available perturbations. FIG. 30E shows a translation example with the histogram of W2 distances for the translation from the starting perturbation of S1172 to the target perturbation of S1628, where the translations using both the fitted perturbation representation and the target drug treatment S1628 have W2 distances smaller than almost all of the W2 distances between the target latent space and the translated latent space via the 158 observed drug treatments. For the LINCS-Drug data, some embodiments also randomly sampled 2000 observed drug treatments to compute the distribution of W2 distances between the target latent space and the translated latent space, and show a translation example in FIG. 30F. Of the 2000 translations, 28.6% have W2 distances smaller than the target W2, and none of the 2000 translations has a W2 distance smaller than the fitted W2.

Discrete Optimal Translation for Optimal Perturbation Selections

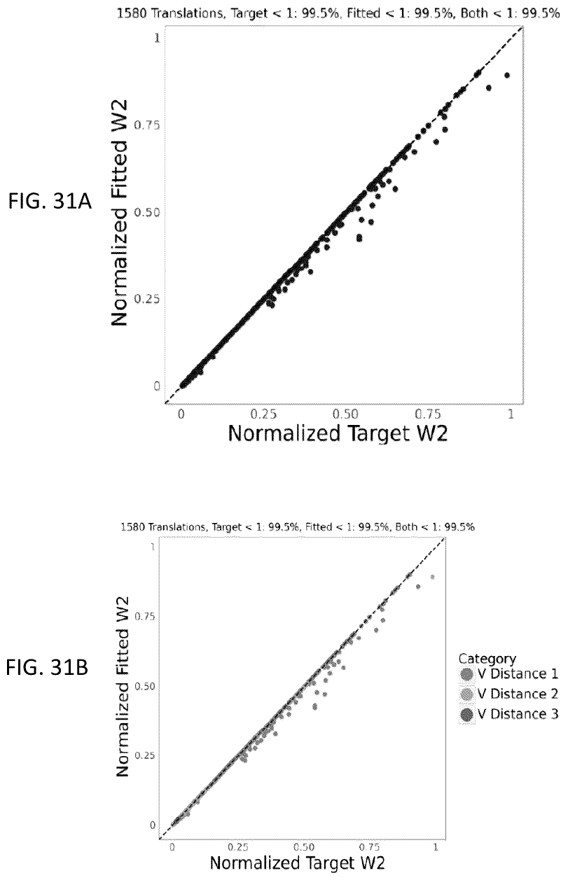

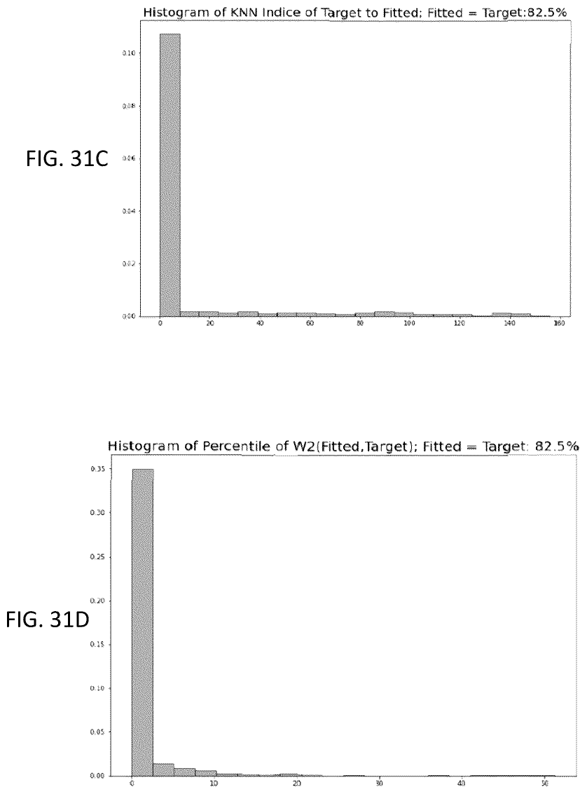

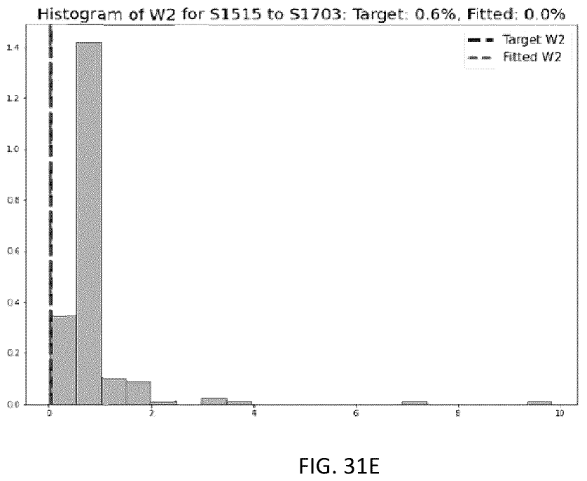

Some implementations performed discrete optimal translations on the sci-Plex and LINCS-Drug data to select the optimal drug treatment to translate a starting latent space to approximate a target latent space. For the sci-Plex, some embodiments randomly selected 10 observed drug treatments as the set of starting perturbations and considered the 158 observed drug treatments as the set of target perturbations. Some implementations implemented the discrete optimal translation algorithm on each pair of a starting and target perturbations. FIGS. 31A-31E show examples of the 1580 discrete optimal translations of the sci-Plex data. The normalized fitted W2 and normalized target W2 are overall the same, both shrinking the original latent distance in around 99.5% of the translations. Because the starting latent space and target latent space in each translation might possess different distributions of residual representation V, some embodiments also computed the W2 distances of residual V's between the two latent spaces across the 1580 translations, and categorized the W2 distances to three tertiles (FIG. 31B). Most of the translations are with the smallest tertile of residual distance (V Distance 1) and do not show an obvious difference from the translations with other tertiles in their normalized fitted and target W2 distances. Some embodiments also trained a KNN algorithm on the observed treatments and evaluated the nearest neighbor index of the target perturbation to the fitted perturbation in each translation. Some implementations show KNN indices of the target perturbation to the fitted perturbation in FIG. 31C and also computed the percentile of the W2 distance between the latent spaces of real cells treated by the target and the fitted perturbations in the distribution of the W2 distances between the target perturbation and other perturbations (FIG. 31D). Of the 1580 translations, 82.5% were selected with the target perturbation as the fitted perturbation, whose KNN indices and W2 distances were 0's. FIG. 31E shows a discrete optimal translation example with the W2 distances between the target latent space of cells treated by S1703 and the translated latent space from a starting perturbation of S1515, using each of the 158 observed drug treatments. The fitted W2 has the smallest W2 among all the translations and 0.6% of the translations have smaller W2 distances than the target W2.

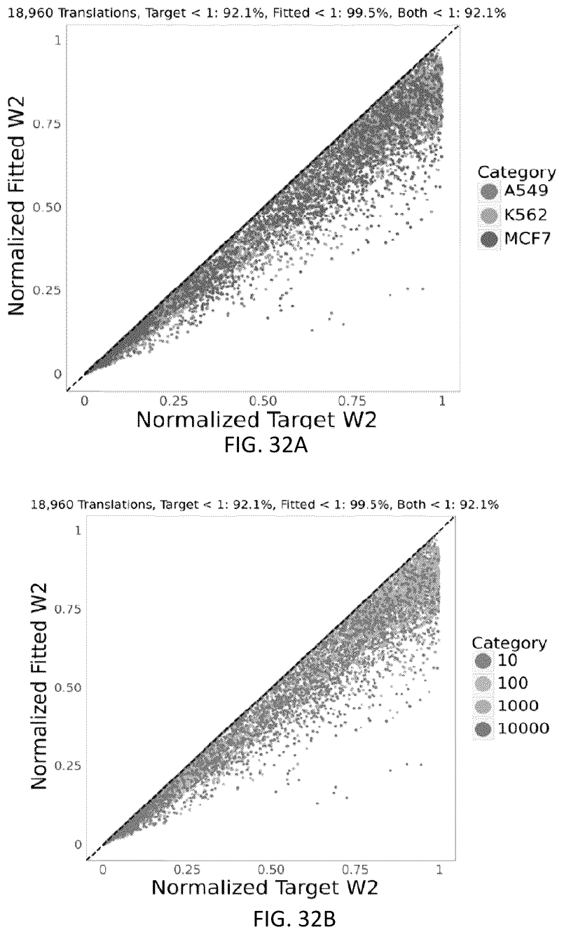

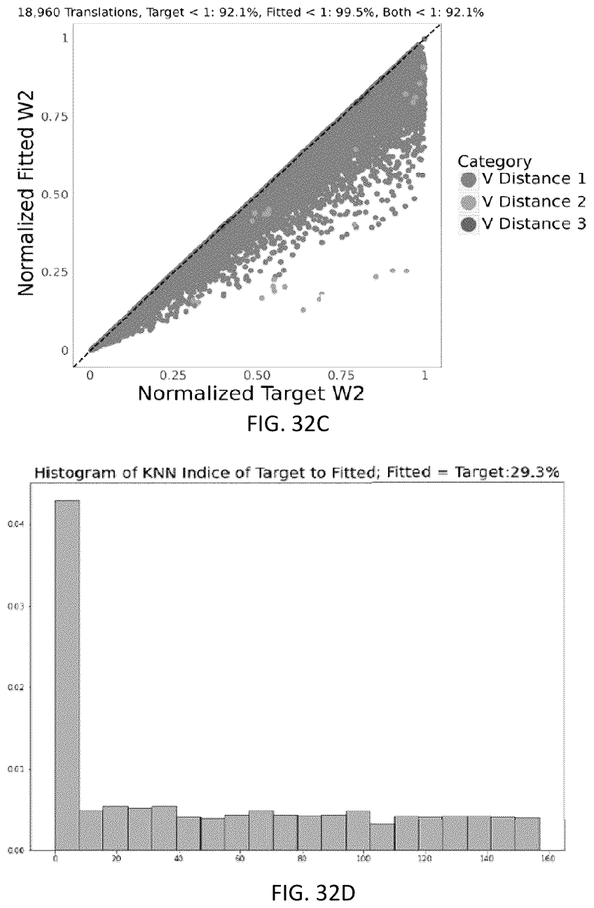

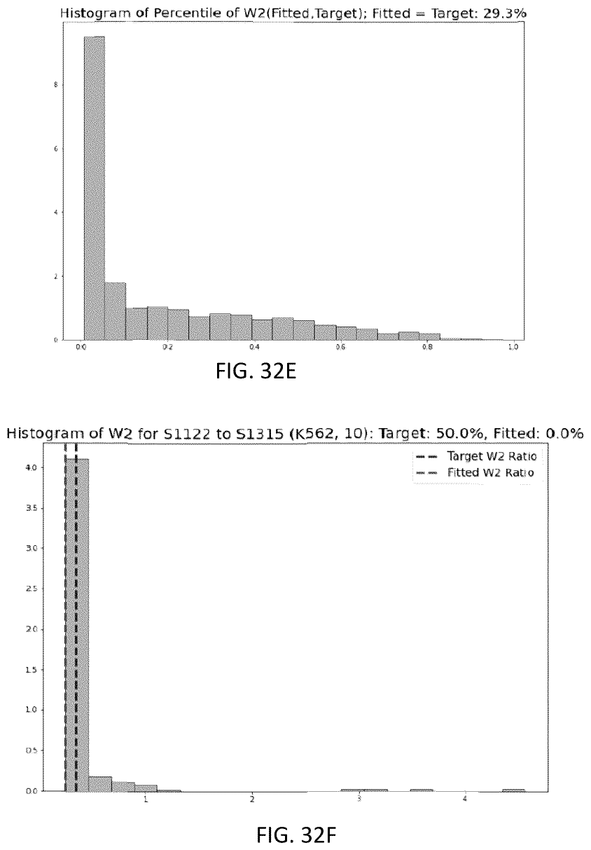

Some implementations also considered translation optimizations of the sci-Plex data in each cell type by dose stratum, and employed the PerturbNet adjusting for cell state covariates to perform the discrete optimal translations between each pair of the 10 starting perturbations and the 158 target perturbations. FIGS. 32A-32C show the normalized fitted W2 and normalized target W2 of the 18,960 translations by cell type, dose and residual W2 distance tertile. The comparison between the normalized fitted W2 and normalized target W2 remains similar across different cell types or doses. Most of the translations have residual distances in the smallest tertile (V Distance 1), while the translations with residual distances in the second tertile tend to have smaller fitted W2 than target W2. Around 29.3% of the translations select the target perturbation as the fitted perturbation (FIGS. 32D-32E). Finally, as an example, FIG. 32F shows the histogram of the W2 distances between the target latent space of S1315 and the translated latent space from a starting drug treatment of S1122 to each of the 158 observed drug treatments with cell type K562 and dose 10. The fitted W2 has a much better performance than the target W2 that is larger than around half of the translations.

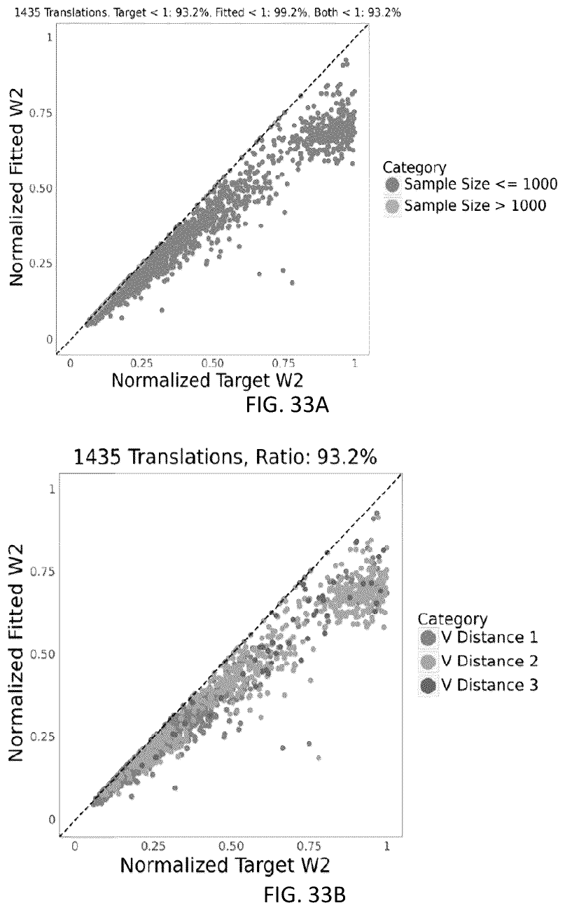

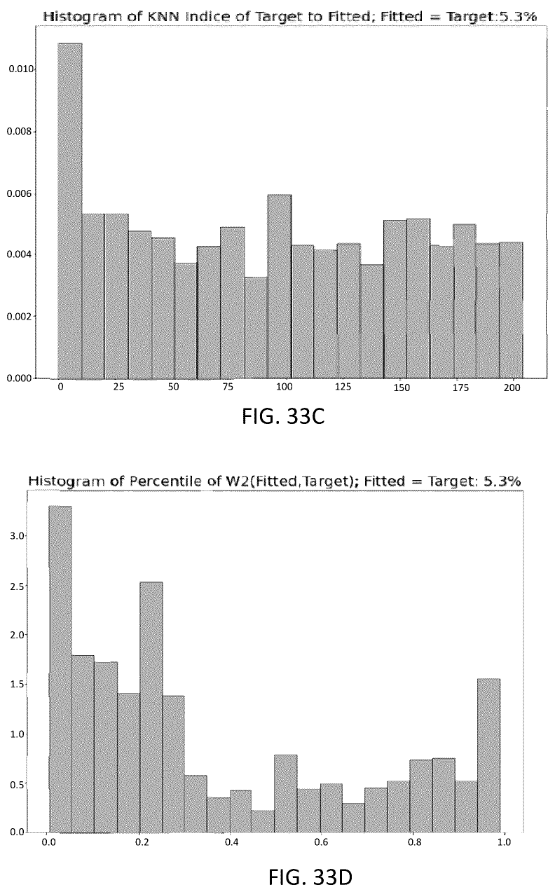

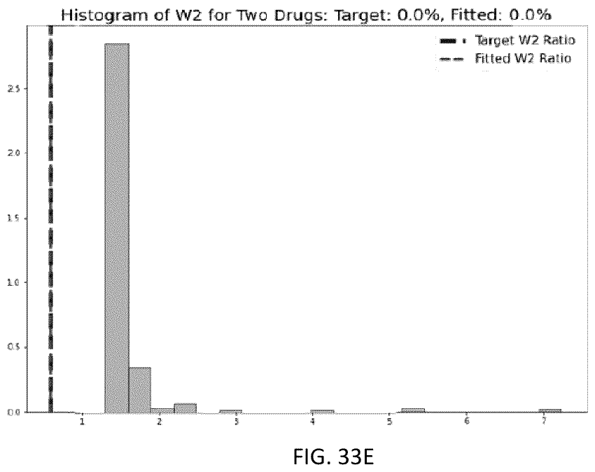

Some embodiments sampled 200 observed drug treatments from the LINCS-Drug data for continuous optimal translations, and these perturbations had small numbers of cells (<200). Thus, some implementations selected another five drug treatments with relatively large numbers of cells (>1000). Some implementations utilized the five drug treatments and another two drug treatments in the previous 200 treatments as the set of starting perturbations. Some implementations considered the combined set of 205 drug treatments as the set of target perturbations. FIGS. 33A-33E show example discrete optimal translations of the LINCS-Drug data. More specifically, FIG. 33A shows the normalized fitted W2 and normalized target W2 by whether both the starting and target perturbations have large numbers of cells (>1000). The translations with large numbers of cells tend to give close normalized fitted W2 and normalized target W2. FIG. 33B shows the normalized fitted W2 and normalized target W2 by residual distance tertile, and most of the translations have small to medium residual distances. Only 5.3% of the 1435 translations select the target perturbation as the fitted perturbation (FIG. 33C), and the latent W2 distance from target perturbation to the fitted perturbation is likely to be smaller than distances from the target perturbation to other perturbations (FIG. 33D). FIG. 33E shows a discrete optimal translation example between two drugs in LINCS-Drug, where the fitted perturbation is different from the target perturbation, and both give percentiles close to zero in the histogram of W2 distances between the target latent space and translated latent space using each of the 205 drug treatments.

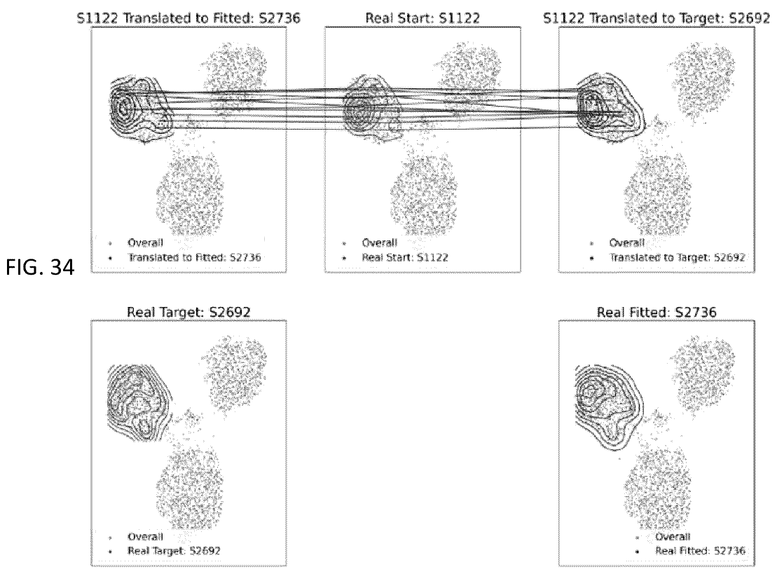

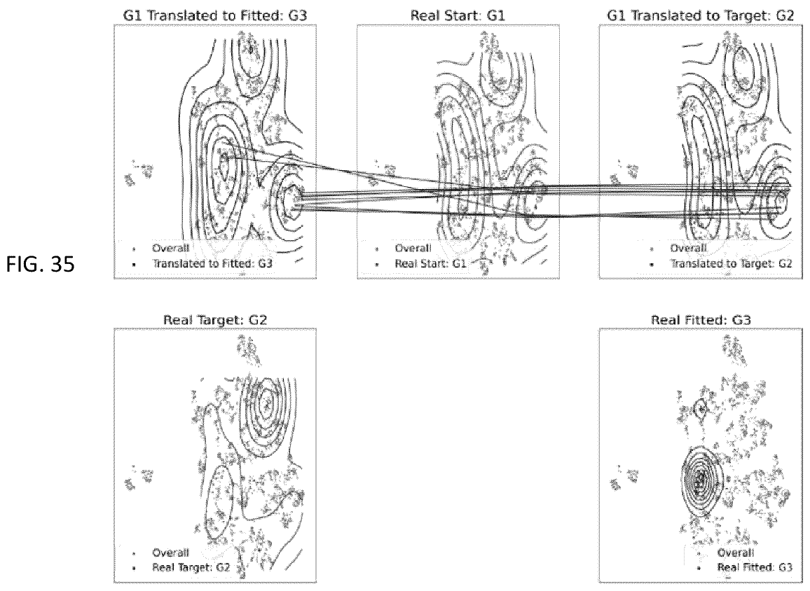

FIG. 34 shows the UMAP plots of starting latent space, target latent space and translated latent spaces of a stratified discrete optimal translation example of the sci-Plex data. The starting latent space of K562 cells treated by S1122 with dose 100 has a different latent distribution from that of the K562 cells treated by S2692 with dose 100. The starting latent space changes slightly after being translated to latent spaces using the target treatment S2692 or the fitted treatment S2736. Based on the W2 distance, the translated latent space using the fitted perturbation S2736 is closer to the target latent space than the one using the target perturbation. FIG. 35 shows the discrete optimal translation example from FIG. 33E for the LINCS-Drug data. The cells treated by the starting perturbation G1 are translated to different latent distributions using the fitted perturbation G3 and the target perturbation G2. The translated latent distribution using the fitted perturbation G3 is closer to the target latent space, and differs from the real latent distribution treated by the fitted perturbation G3.

Perturbation Attributions of Cell States for Atomic Scores

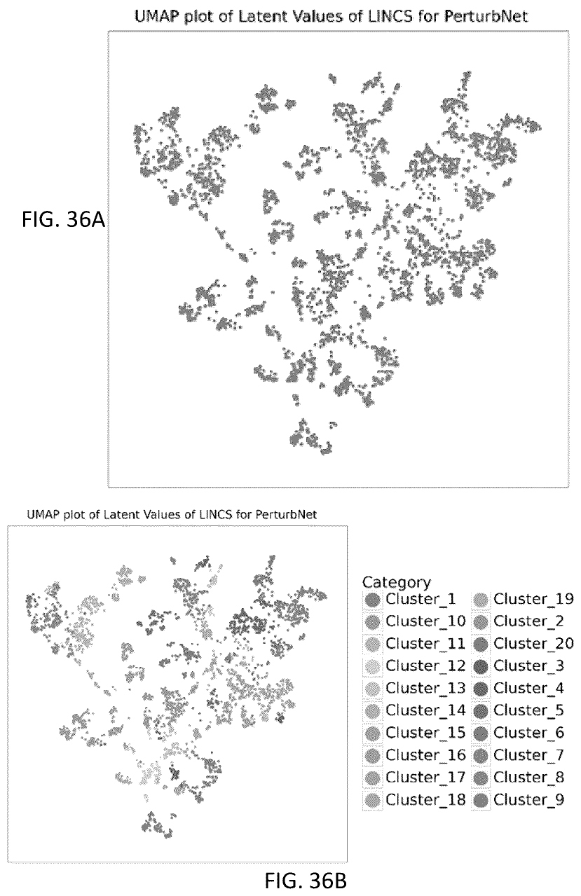

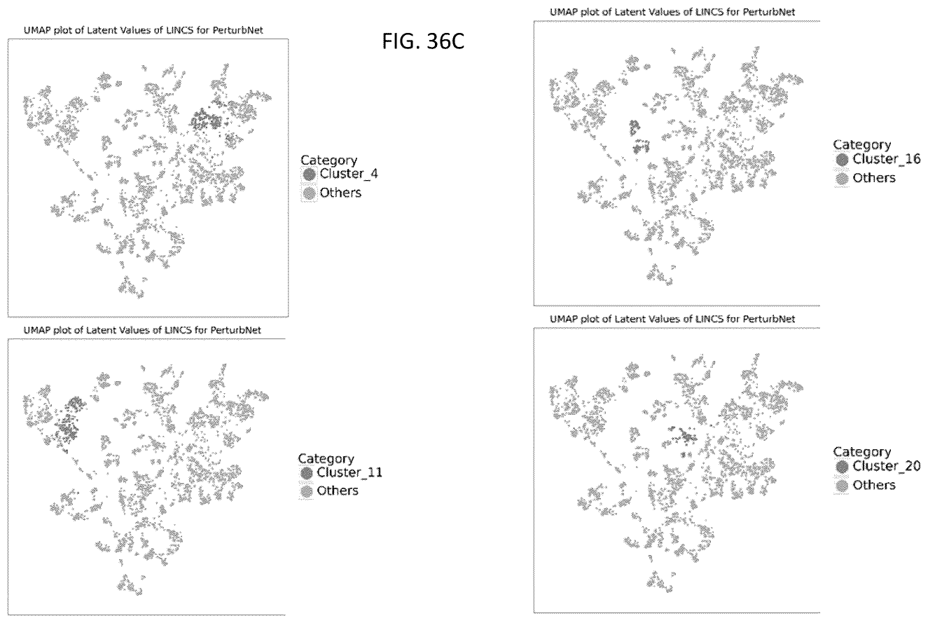

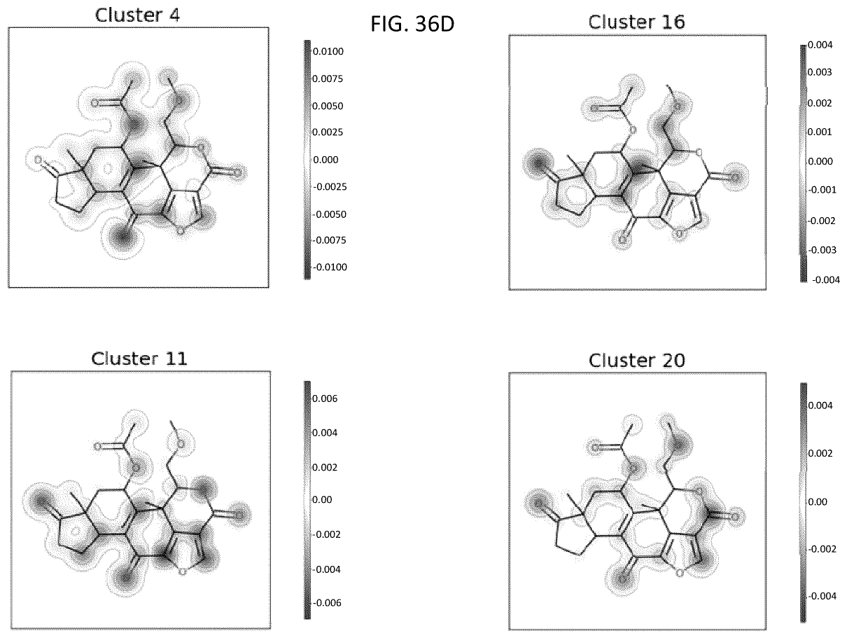

Some implementations utilized the model interpretability method of integrated gradients to interpret how a perturbation shapes a cell state. Some implementations performed k-means clustering on the latent values of VAE trained on the LINCS-Drug data. Some implementations calculated the average silhouette widths for a range of cluster numbers, and identified 20 as the optimal number of clusters (FIGS. 36A-36B). Some implementations trained a neural network model to classify latent values into each of these 20 clusters. Some implementations then interpreted the features of a chemical perturbation in contributing to the probability of generating cells in a particular cluster.

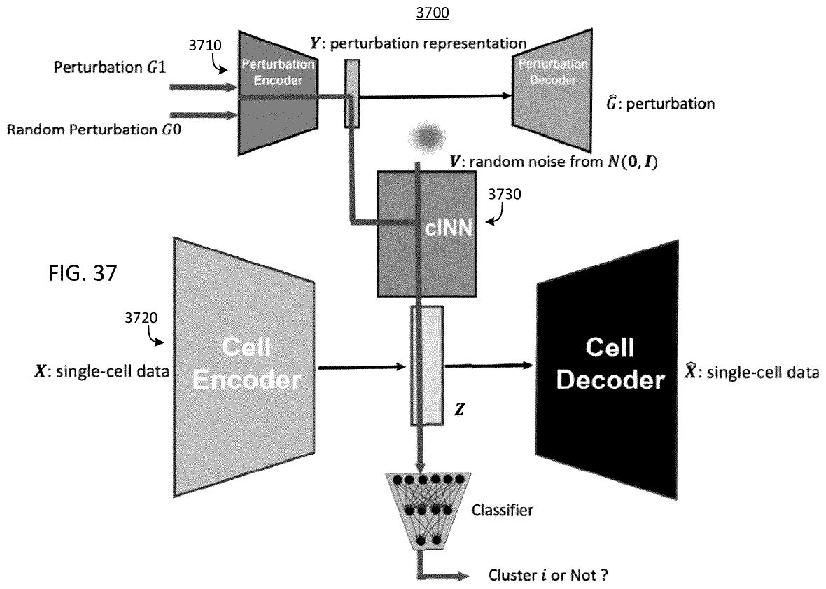

Some implementations utilized an observed drug treatment G1 in the LINCS-Drug dataset and also selected a random observed drug treatment G0. Some implementations employed both G1 and G0 with the same sampled V from the standard normal prior distribution to translate to cellular representations, which some implementations further treated as inputs to the latent classifier model to obtain their probabilities of being classified to a cluster. Some implementations used the integrated gradients method to interpret the attributions of features of G1 to contribute to the probability of generating cells in a latent cluster based on the baseline input G0. FIG. 37 illustrates an example model interpretation procedure on a perturbation to impact the formation of a latent cluster. Some implementations replicated this model interpretation procedure 3000 times with a fixed G1 and a random G0 in each replication, and then averaged the attributions of G1 across the replications.

After some obtaining the average feature attributions of G1 for each cluster, some implementations utilized the SimilarityMaps package (Riniker, S., and G. A. Landrum (2013), Similarity maps-a visualization strategy for molecular fingerprints and machine-learning methods, Journal of cheminformatics, 5(1), 1-7) to visualize the molecular structure of G1 with its atoms colored by their attributions. Some implementations utilized four clusters of the LINCS-Drug data (FIG. 36C) with test-set accuracy values of {99.78%, 99.86%, 99.93%, 99.97%} from the classification model. FIG. 36D shows the annotated molecular structures of G1 for the four latent clusters. The atomic attributions show atoms that increase the probability of generating a particular cell state cluster (green) or decrease the probability (red). The atoms of G1 have varying contributions to the probabilities of generating cells in different clusters.

More broadly, with respect to FIG. 37, the system 3700 may include at least three networks with different functions. More specifically, the first network 3710 may convert perturbations to real-valued vector representations of the perturbations. The second network 3720 may convert cell states into real-valued vector representations of the cell states. And the third network 3730 may map relationships between: (i) the real-valued vector representations of the perturbations, and (ii) the real-valued vector representations of the cell states.

Perturbation Attributions of Cell States for Gene Ontology Scores

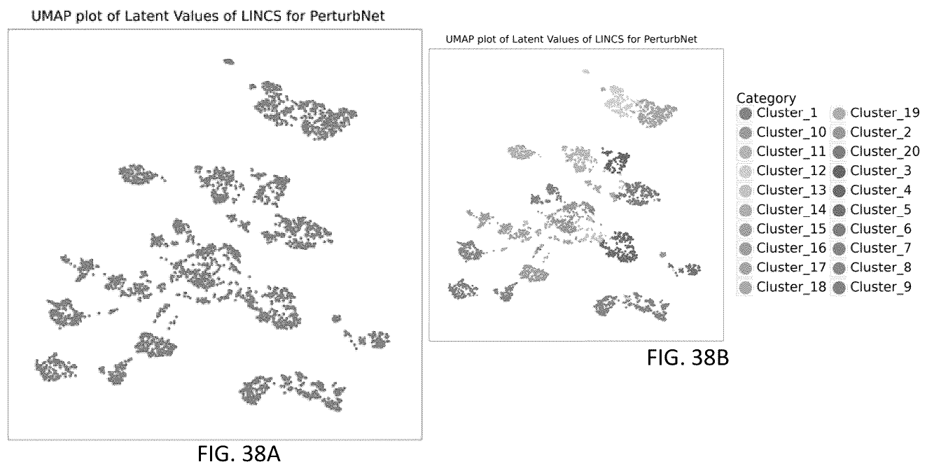

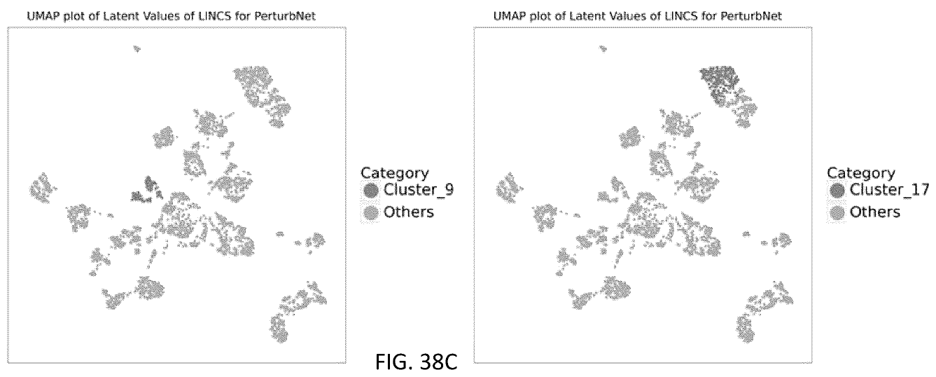

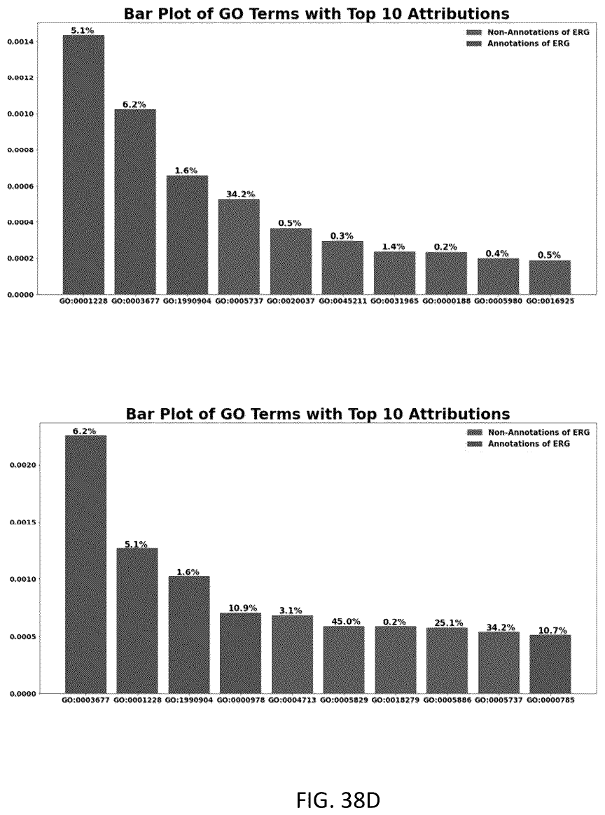

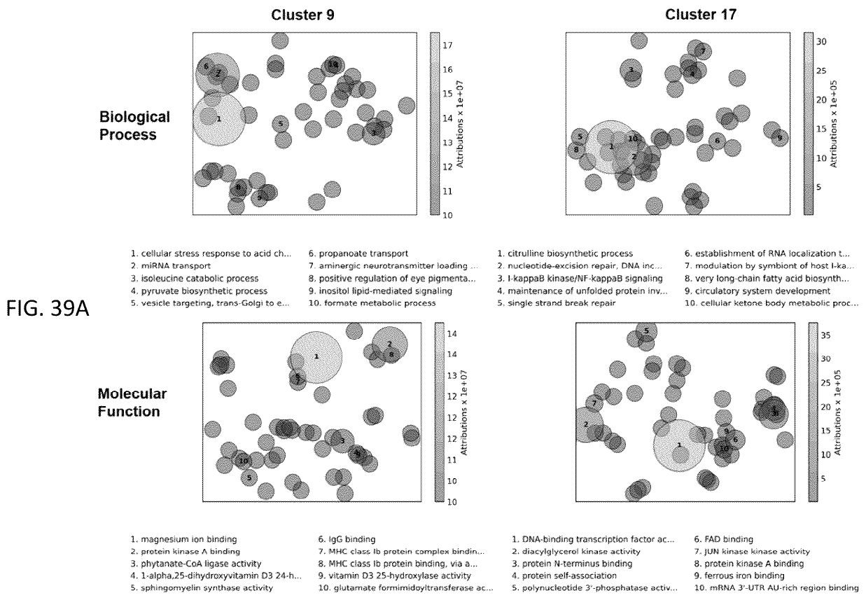

Some implementations also performed a similar analysis with genetic perturbations. Some implementations utilized the integrated gradients method to determine the gene ontology terms that contribute to the probability of generating a particular cell state cluster. As with the drug data, some implementations performed k-means clustering on the latent values of VAE trained on the LINCS-Gene data, calculated the average silhouette widths for a range of cluster numbers, and identified as the optimal number of clusters (FIG. 38A-38B). Some implementations also trained a neural network model to classify latent states into each of these clusters. Following the model interpretation procedure in FIG. 37, some implementations determined the features of a genetic perturbation that increased or decreased the probability of generating latent cell states classified as a particular cluster.

Some implementations selected an observed genetic perturbation (knockdown of the gene ‘ERG’) and a random observed genetic perturbation as input and baseline. Some implementations also replicated the interpretation 3000 times and averaged the attributions across these replications. Some implementations then mapped the attributions to the specific G0 terms with which ERG is annotated. Some implementations show the UMAP plots of two latent clusters 9 and 17 whose test-set accuracy values are {99.91%, 99.89%} from the classification model (FIG. 38C), and the G0 terms with the 10 highest attributions for the two clusters (FIG. 38D). The two annotations for DNA binding (‘G0:0001228’ and ‘G0:0003677’) of the ERG gene have the highest attributions to generate latent values in cluster 9 and 17. These two annotations are present in 5.1% and 6.2% of the baseline genetic perturbations, respectively.

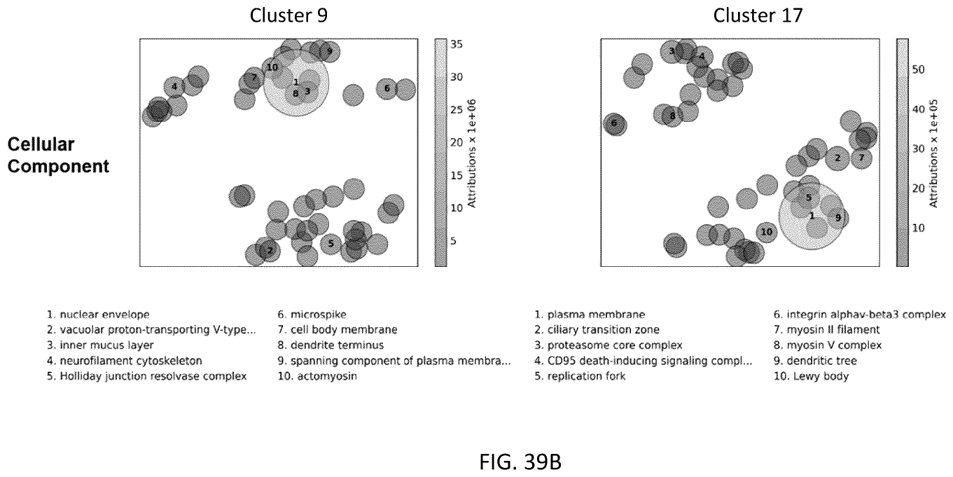

Some implementations further visualized the attributions of the G0 annotations of ERG in biological process, molecular function and cellular component following a similar procedure to the G0 enrichment analysis conducted in one study (Lu, L., and J. D. Welch (2022), PyLiger: scalable single-cell multi-omic data integration in Python, Bioinformatics, doi:10.1093/bioinformatics/btac190, btac190). Some implementations performed multidimensional scaling of the GO terms and plot GO terms as circles with both color and size indicating the attribution values (FIGS. 39A-39B). Some GO terms are related to the transcription factor activity of ERG, as shown by the DNA-binding annotations with high attributions in FIG. 38D.

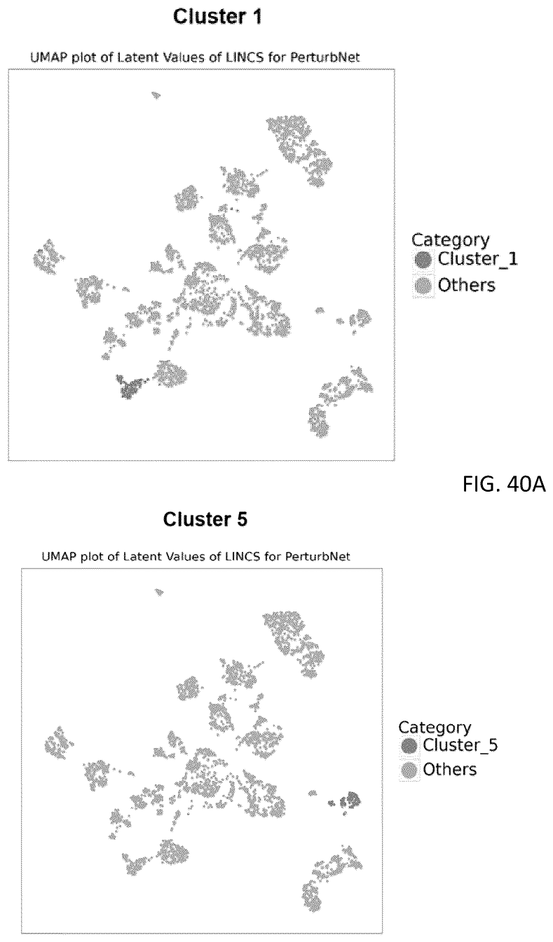

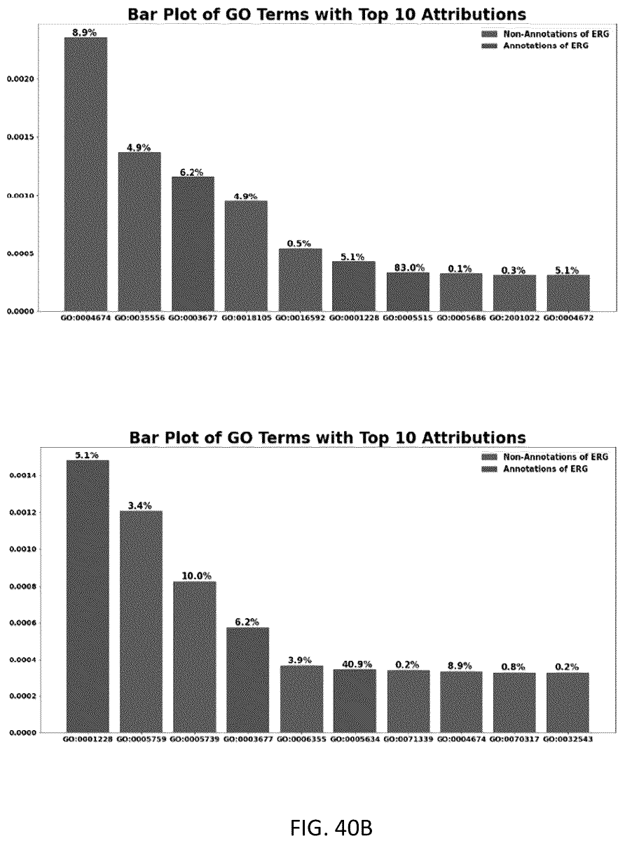

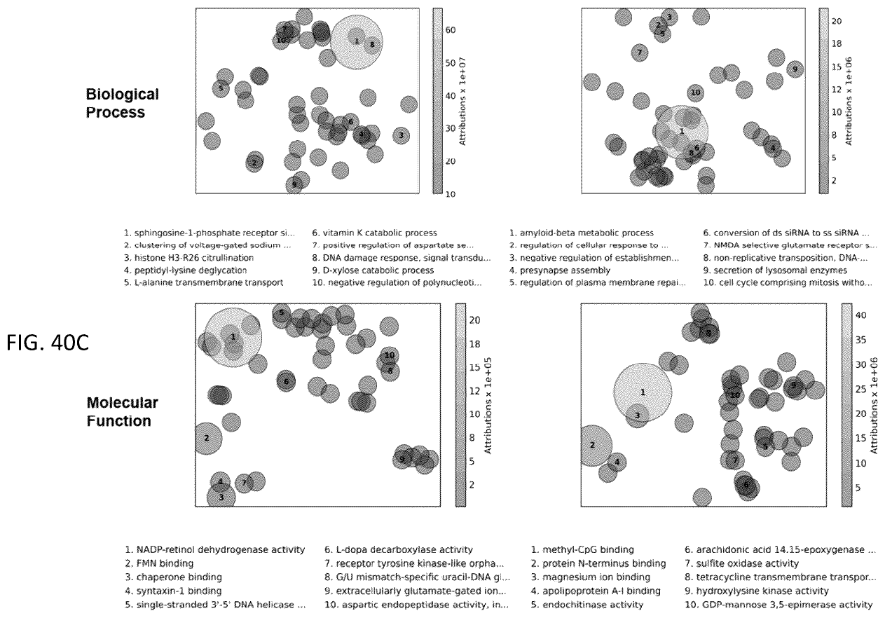

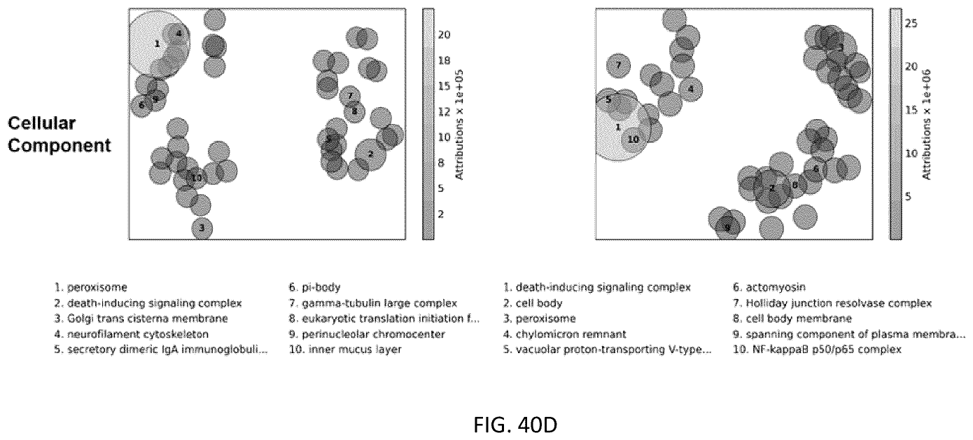

Some implementations also evaluated the attributions of GO terms of ERG for the latent clusters 1 and 5 whose test-set accuracy values are {99.96%, 99.97%} from the classification model in FIG. 40A. The protein serine kinase/threonine activity annotation of ‘GO:0004674’ is not an annotation of ERG but is present in 8.9% of the baseline perturbations. This annotation gives a negative contribution to generate latent values in cluster 1. The DNA-binding transcription activator activity annotation of ‘GO:0001228’ is only in 5.1% of the baseline perturbations and is the most important annotation for ERG to the formation of cluster 5 (FIG. 40B). The plots of GO terms also reflect the transcription factor activity of ERG for cluster 1 and 5 (FIGS. 40C-40D).

Perturbation Attributions for Optimal Translations

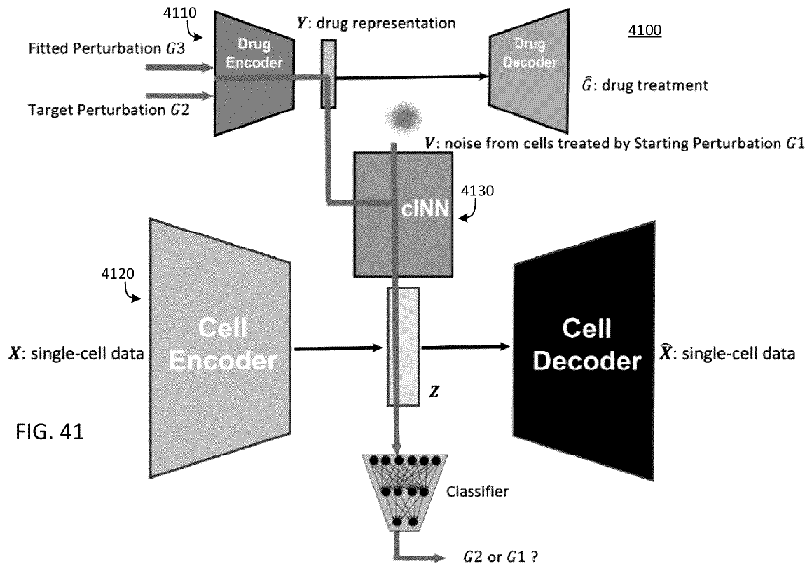

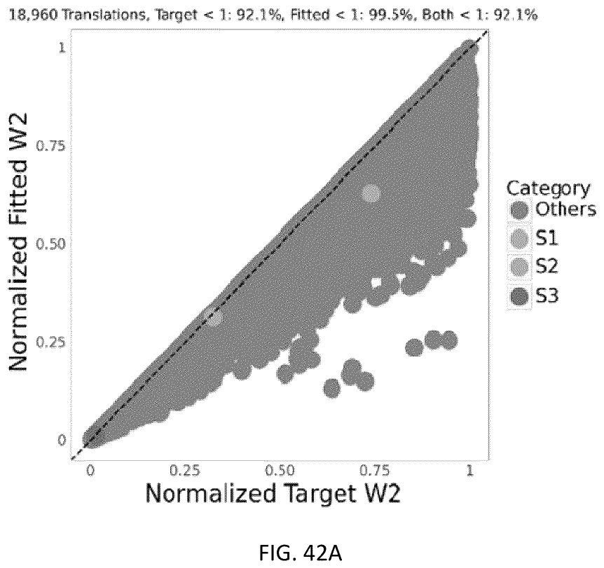

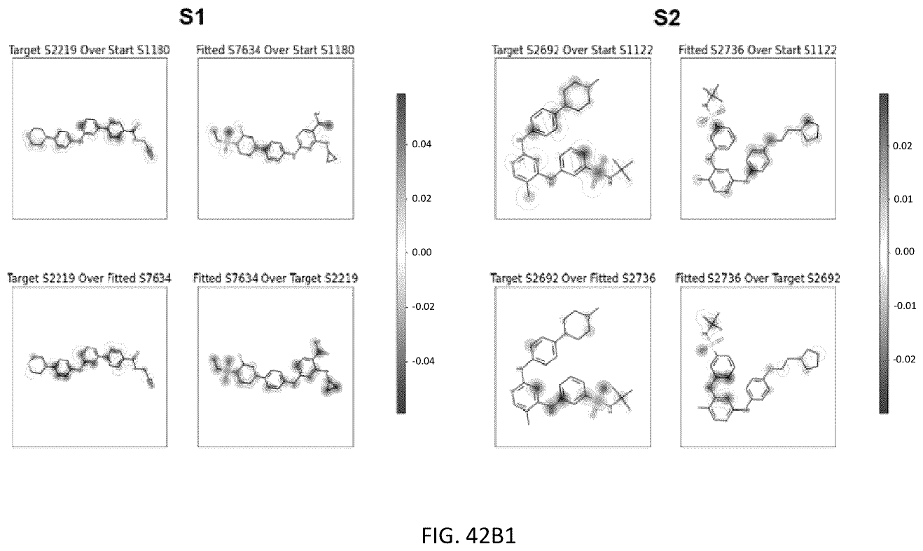

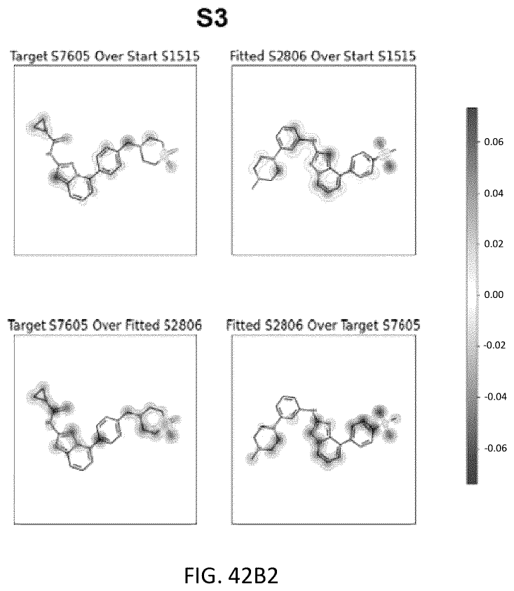

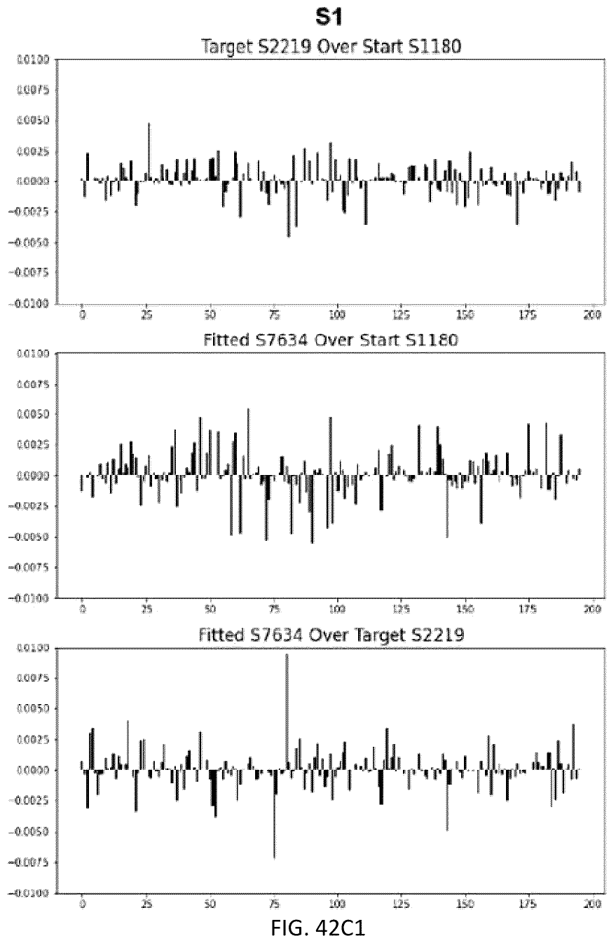

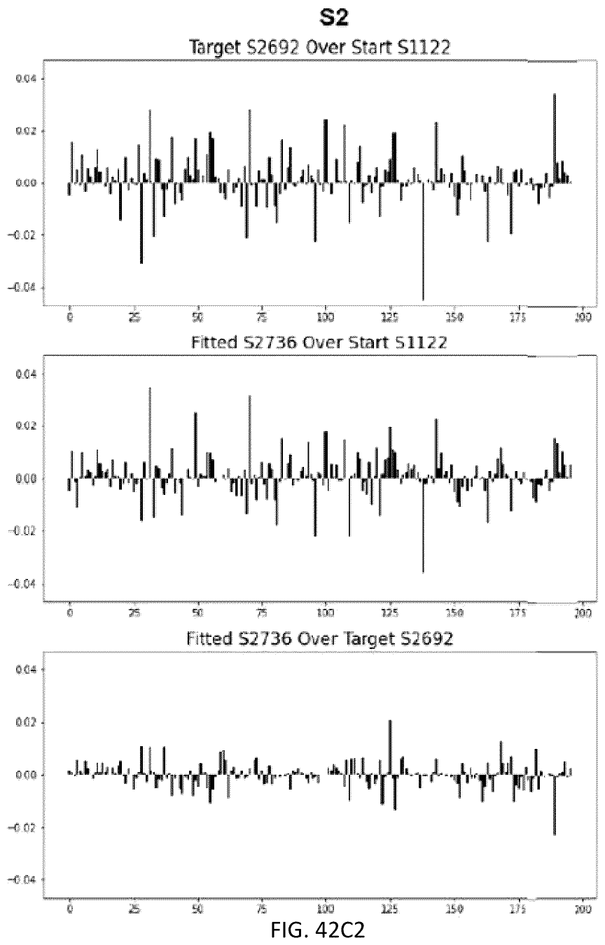

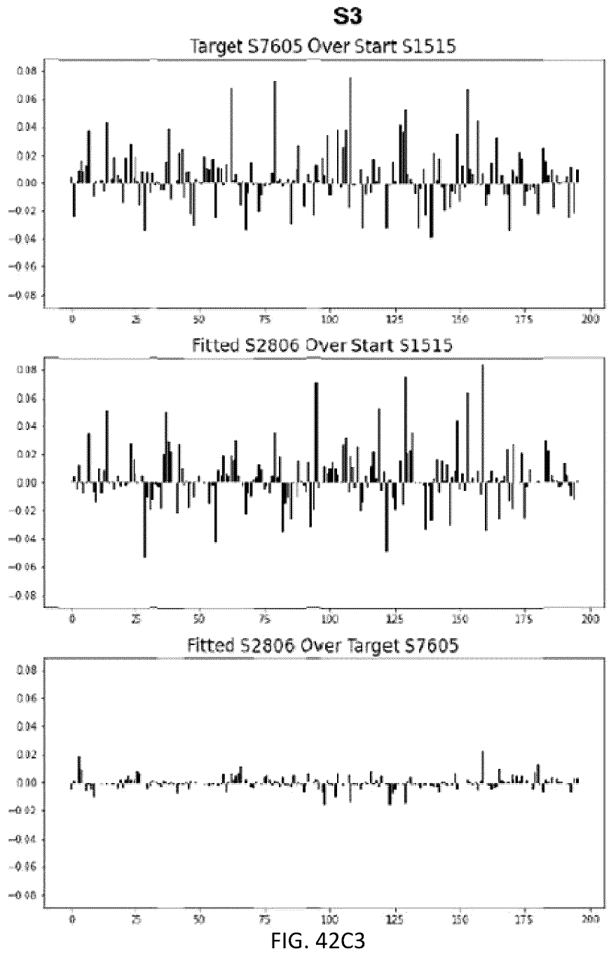

Some implementations used the integrated gradients method to interpret the discrete optimal translations described. Some implementations calculated the attributions of fitted and target perturbations, compared to a baseline perturbation. Some implementations selected three scenarios of discrete optimal translations of sci-Plex with different fitted and target perturbations in Table 3. For each selected optimal translation, some implementations have a starting latent space with cells of a certain cell type treated by a starting perturbation at a certain dose, as well as a target latent space with cells of the same cell type and dose but a different perturbation. The fitted perturbation is the optimal perturbation obtained in discrete optimal translations, and FIG. 42A shows the normalized fitted W2 and normalized target W2 of the three selected scenarios of the sci-Plex. Some implementations trained a neural network model on the latent values in the starting and target latent spaces to classify cell state to the target latent space with test-set accuracy values being {56.76%, 69.23%, 98.81%}. Some implementations then interpreted a perturbation to translate to the target cell state using the V's from the starting latent space. FIG. 41 shows the model interpretation procedure for discrete optimal translations. This model interpretation procedure evaluates the performance of a perturbation to translate the cells in the starting latent space to approximate the target latent space in a translation. Some implementations interpreted the perturbation features of the one-hot matrix B and of the perturbation representation Y.

More broadly, with respect to FIG. 41, the system 4100 may include at least three networks with different functions. More specifically, the first network 4110 may convert perturbations to real-valued vector representations of the perturbations. The second network 4120 may convert cell states into real-valued vector representations of the cell states. And the third network 4130 may map relationships between: (i) the real-valued vector representations of the perturbations, and (ii) the real-valued vector representations of the cell states.

TABLE 3
Selected Scenarios of Discrete Optimal Translations
of the sci-Plex and LINCS-Drug data.

| | | | Cell | | | | |
|---|---|---|---|---|---|---|---|
| Dataset | Notation | Start | Type | Dose | Target | Fitted | Accuracy |
| sci-Plex | S1 | S1180 | A549 | 10 | S2219 | S7634 | 56.76% |
| | S2 | S1122 | K562 | 100 | S2692 | S2736 | 69.23% |
| | S3 | S1515 | MCF7 | 10000 | S7605 | 2806 | 98.81% |
| LINCS- | Scenario | G1 | — | — | G2 | G3 | 86.36% |
| Drug | | | | | | | |

For each of the three selected sci-Plex scenarios {S1, S2, S3}, some implementations interpreted the input and baseline pairs of {Target, Start}, {Fitted, Start} and {Fitted, Target}. Some implementations obtained the average feature attributions of one-hot matrix B or perturbation representation Y of fitted and target perturbations. FIGS. 42B1-42B2 show molecular structures of fitted and target perturbations colored by their atomic attributions based on the starting perturbation, along with their structures colored by fitted-target atomic attributions.

For each scenario, the fitted perturbation has some similar components as the target perturbation, and they possess different atomic scores to translate the starting cell state to the target cell state. Both S2 and S3 show that the fitted and target perturbations have some atoms with positive attribution scores based on the starting perturbations and have their atomic scores attenuated or diminished when compared with each other. This phenomenon reflects the fact that the target and fitted perturbation have similar performances in shrinking the original latent distances for S2 and S3 in FIG. 42A. The attenuation of atomic scores of fitted and target perturbations of S1 is less obvious, as its fitted perturbation outperforms the target perturbation in shrinking the latent distance. FIG. 42C1-42C3 show the interpretation of the feature attributions for optimal translations on perturbation representations Y. In each scenario, the target and fitted perturbations have distinctive attributions of perturbation representations based on the starting perturbation. However, the attributions of the fitted perturbation compared with the target perturbation in each scenario show that each dimension diminishes in magnitude, especially for S3, whose translations using the fitted and target perturbation both effectively approximate the target latent space. The attributions comparing the fitted and target perturbation of S2 have also been slightly shrunk, while those of S1 do not change significantly.

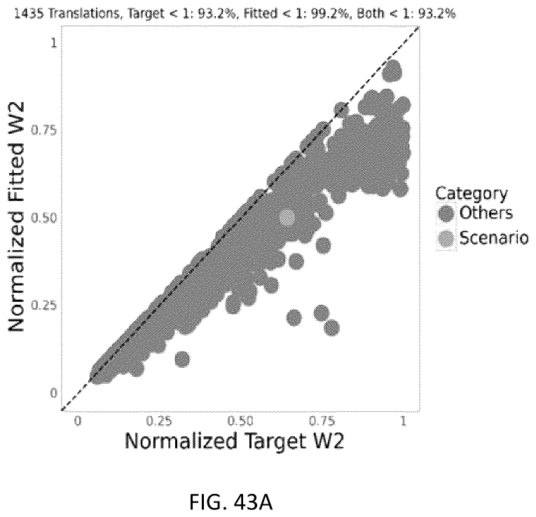

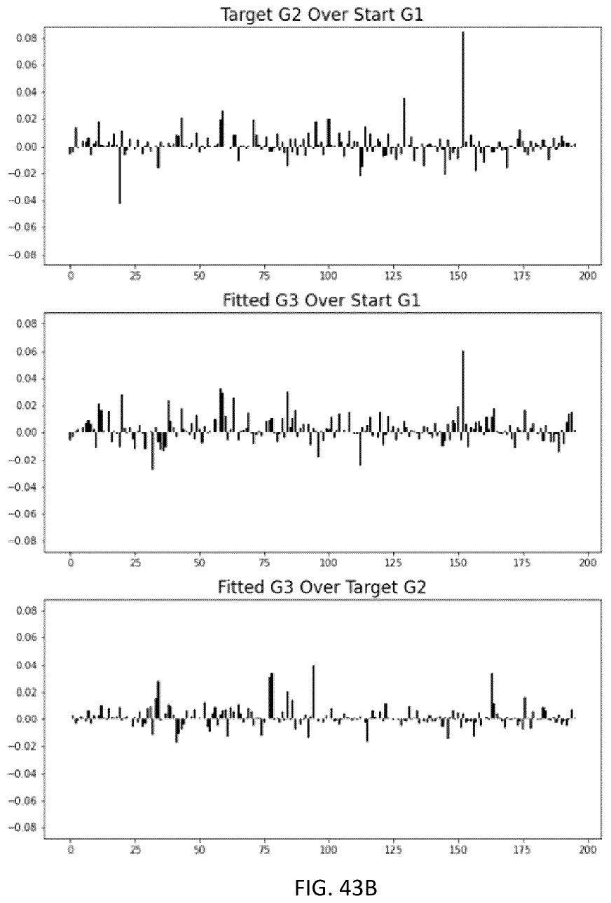

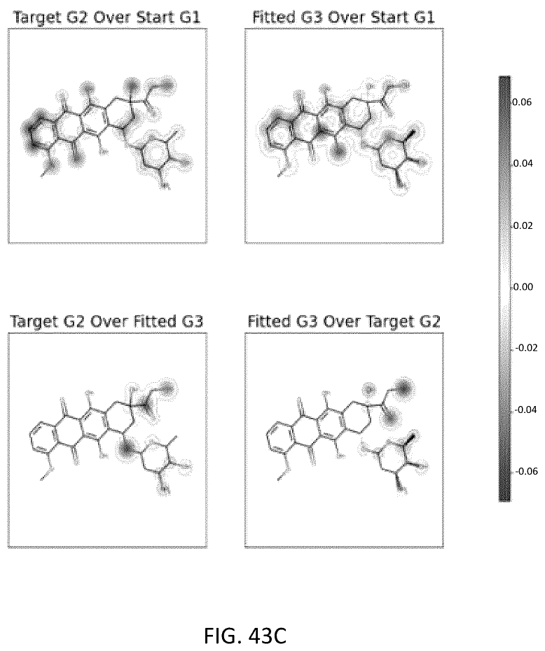

Some implementations also interpreted one scenario of the discrete optimal translations for the LINCS-Drug data, as shown in Table 3. The classification model gave the test-set accuracy of 86.36%. The fitted perturbation G3 has a slightly better performance in shrinking the original latent distance than the target perturbation G2 (FIG. 43A). FIG. 43B shows that when G1 serves as the baseline perturbation, the fitted perturbation G3 has overall higher attributions of perturbation representation than target perturbation G2. It also shows that G2 attenuates the attributions of G3. The molecular structure of G3 has more atoms than G2 to show positive attributions to translate the starting latent space to the target latent space, with G1 as the baseline perturbation (FIG. 43C).

Perturbation Attributions of Genetic Perturbations for Shifting Cell State Distributions

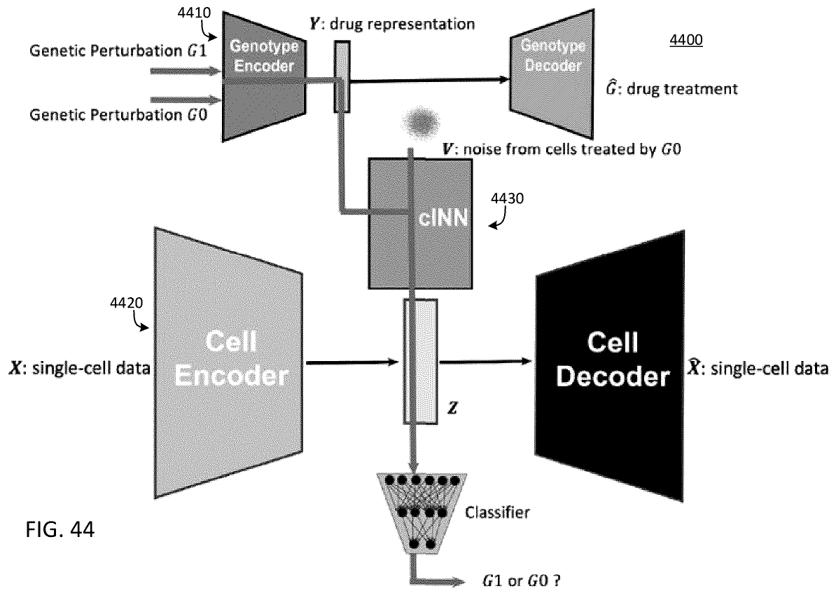

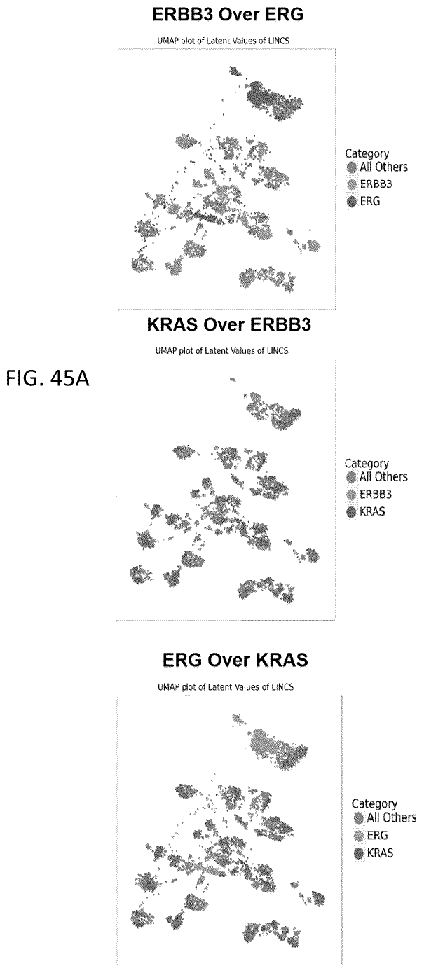

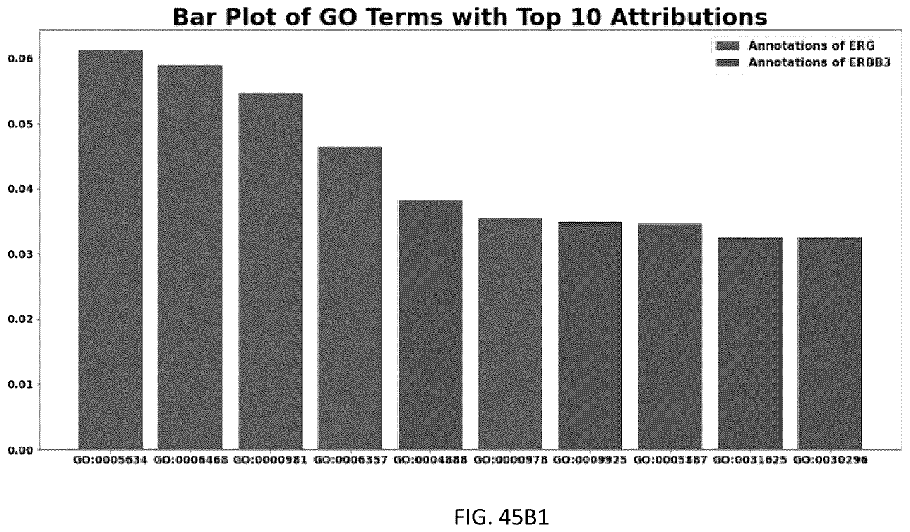

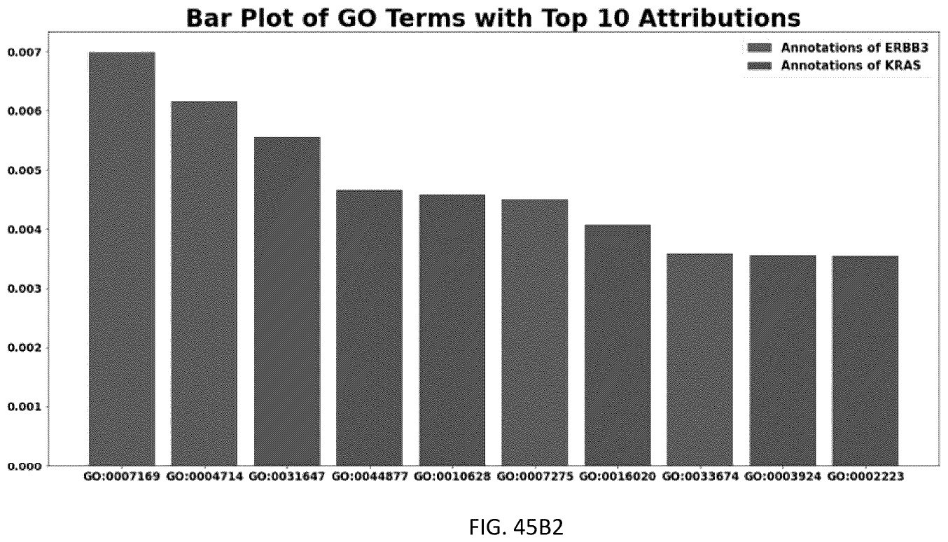

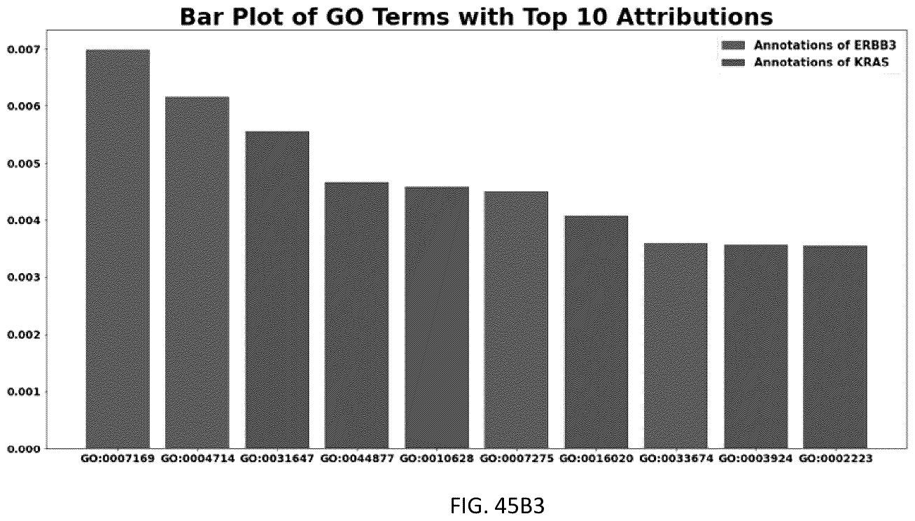

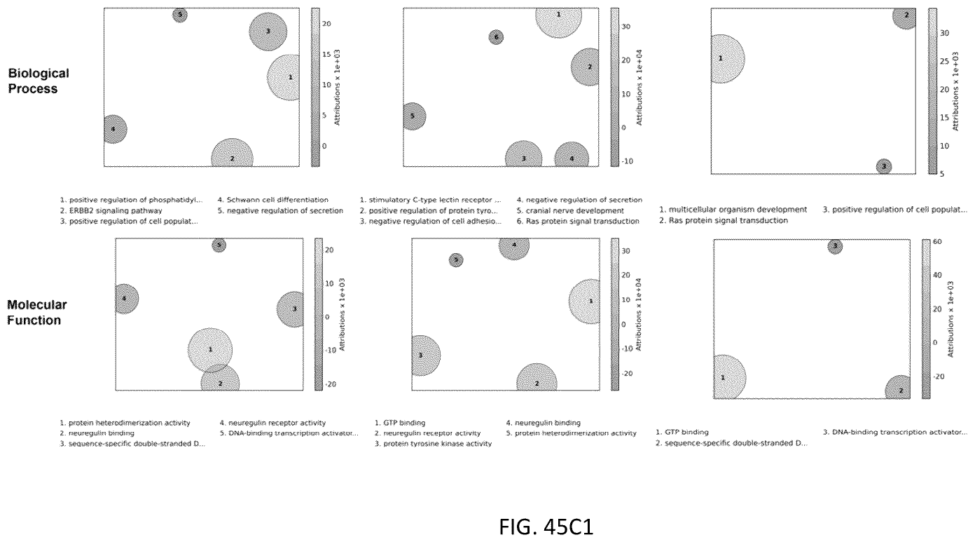

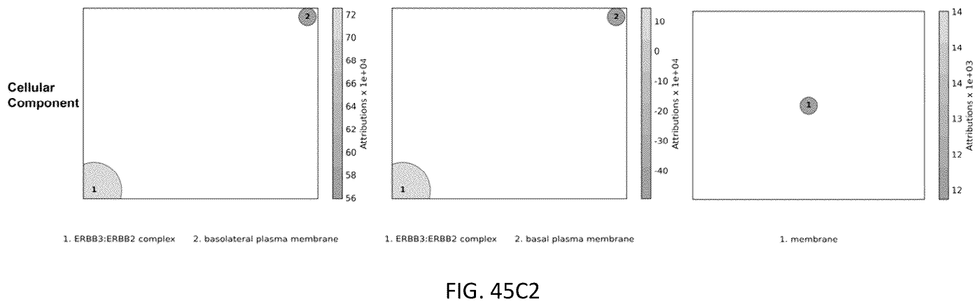

Some implementations also interpreted the attributions of the features of a genetic perturbation to forming its cell state distribution, compared to the cell state distribution of another perturbation. Some implementations selected three pairs of genetic perturbations including {(ERG, ERBB3), (ERBB3, KRAS), (KRAS, ERG)} in FIG. 45A. Some implementations denoted each pair as (G0, G1), and trained a neural network model on the latent values of cells treated by G0 or G1 to classify their cell state to latent space of G1. Some implementations utilized G1 and G0 to translate the cells treated by G0 to new cell state, and then obtained probabilities of being classified as G1 latent space. FIG. 44 summarizes the model interpretation procedure of pairs of genetic perturbations for shifting cell state distributions. The classification models for the three pairs gave the test-set accuracy values of {92.51%, 62.81%, 93.7%}.

More broadly, with respect to FIG. 44, the system 4400 may include at least three networks with different functions. More specifically, the first network 4410 may convert perturbations to real-valued vector representations of the perturbations. The second network 4420 may convert cell states into real-valued vector representations of the cell states. And the third network 4430 may map relationships between: (i) the real-valued vector representations of the perturbations, and (ii) the real-valued vector representations of the cell states.

Some implementations obtained the feature attributions of G0 annotation vector of G1 compared to that of G0 in shifting the cells treated by G0 to cells treated by G1. The common genetic annotations between G1 and G0 do not provide different feature input and thus have attributions of 0's. FIGS. 45B1-45B3 show the uncommon annotations between G1 and G0 with the 10 highest attributions. Not having ERG annotations of nucleus (‘GO:0005634’), protein phosphorylation (‘GO:0006468’), DNA-binding transcription factor activity (‘G0:0000981’) and regulation of transcription by RNA polymerase II (‘GO:0006357’) attributes the most for ERBB3 to shift the original ERG cell state to the ERBB3 cell state. Not having transmembrane receptor protein tyrosine kinase signaling pathway annotation (‘GO:0007169’) and having protein phosphorylation annotation (‘GO:0006468’) for ERG gives the highest attributions to change the cell states of ERBB3 and KRAS to those of KRAS and ERG, respectively.

FIGS. 45C1-45C2 show the GO terms using the attributions in shifting a cell latent space to another cell latent space. Several GO terms give meaningful interpretations of shifting the cell states. For example, the GO term for ERBB2 signaling pathway in biological process has a high attribution to shift ERG cells to ERBB3. In addition, the ‘ERBB3:ERBB2’ annotation in cellular component has a strong signal of distinguishing two genetic perturbations in the pairs of (ERBB3, ERG) and (KRAS, ERBB3).

### DISCUSSION

The following discussion considers designing perturbations from predicted single-cell responses using PerturbNet, and also discovering key components within a chemical or genetic perturbation to the formation of a cell state. The following also proposes two algorithms: continuous optimal translation of perturbation representation, and discrete optimal translation to search for the optimal perturbation to translate a starting cell state to a desired target cell state. Some implementations also employ the integrated gradients method to interpret the feature attributions of a chemical or genetic perturbation in increasing the probability of generating a specific cell state, with its molecular structures colored by atomic scores or its plot of G0 scores. Some implementations also interpret the optimal translation experiments, and the attributions of a genetic perturbation to shift another perturbation's cell state.

Some embodiments propose optimal translation algorithms aim to design an optimal perturbation for individual cells, and can be further utilized to perform research in specific biomedical scenarios. For example, the optimal translation algorithms can discover a suitable drug treatment to change some diseased cells to approximate a healthy cell state. In addition to drug treatments, the optimal translation algorithms can also be utilized to find an optimal genetic perturbation to change genetic functions.

Some experiments disclosed herein perform discrete optimal translations with limited numbers of drugs due to the computational intensity to search through the discrete drug set and to demonstrate the properties of optimal perturbation. In practice with a well-defined perturbation discovery goal, the discrete optimal translations can be implemented in large chemical databases such as PubChem (Kim, S., et al. (2016), Pubchem substance and compound databases, Nucleic acids research, 44(D1), D1202-D1213). In contrast, the continuous optimal translation is more computationally efficient. However, the optimal perturbation representation obtained from the continuous optimal translation needs to be further engineered to design an optimal chemical or genetic perturbation. For example, a possible direction for chemical perturbations is to utilize the chemical variational autoencoder to generate drug treatments from the perturbation representation (Gömez-Bombarelli, R., et al. (2018), Automatic chemical design using a data-driven continuous representation of molecules, ACS central science, 4(2), 268-276). This is an exciting direction for future work.

Atomic Attributions Visualizations

Some implementations employ the SimilarityMaps package (Riniker, S., and G. A. Landrum (2013), Similarity maps-a visualization strategy for molecular fingerprints and machine-learning methods, Journal of cheminformatics, 5(1), 1-7) to draw molecules from canonical SMILES strings. Some implementations extract the atomic attributions in the one-hot perturbation vector using the edited smiview Python package and use them as atomic weights for similarity maps.

Classification Models

The neural network classification models use multilayer perceptron (MLP) units and have a fully-connected (FC) hidden layers with 32 neurons with the Rectified Linear Unit (ReLU) activation, batch normalization, dropout regularization of a dropout probability of 0.1. The output layer has one neuron with the sigmoid activation. Some implementations train the classification models (as described above) for latent clustering with batch Adam optimization, and train the classification models (as also described above) for pairs of perturbations with minibatch Adam optimization with a batch size of 128.

### SUMMARY

Embodiments described herein disclose PerturbNet, a multi-stage deep generative model to predict single-cell responses to drug treatments. The PerturbNet framework consists of a three-stage modeling process with chemical variational autoencoder on drug treatments, a single-cell VAE model on single-cell samples, as well as a conditional invertible neural network (cINN) that connects perturbation representation and cellular representation. Some implementations show that PerturbNet effectively connects perturbation information and cell state. Some implementations demonstrate the excellent prediction performance of PerturbNet, with another proposed KNN model, to predict single-cell responses to either observed or unseen perturbations. By regularizing through properties of cellular representation, some implementations develop an algorithm to fine-tune the learned perturbation representation of Chemical-VAE, and to further enhance the performance of single-cell predictions for unseen perturbations. Some implementations also show the importance of adjusting for cell state covariates to better learn the condition-invariant or residual representations for individual cells and to more accurately generate individual cellular responses through the PerturbNet framework.

This disclosure also extends PerturbNet to predict single-cell responses to genetic perturbations. Some implementations consider two types of genetic perturbations in CRIPSR gene-editing experiments. For the genetic perturbations with target gene identification, some implementations develop a deep generative model called GenotypeVAE to encode the gene ontology (GO) annotation vector of target genes to dense representation, and then decode it to reconstructed vector. For the genetic perturbations with protein-coding sequence variants, some implementations employ a pre-trained start-of-the-art protein transformer model (Rives, A., et al. (2021), Biological structure and function emerge from scaling un-supervised learning to 250 million protein sequences, Proceedings of the National Academy of Sciences, 118(15)) to encode coding variants. Some implementations evaluate KNN and PerturbNet on several high-throughput genetic screen datasets, and show their high prediction performances. Some implementations also fine-tune GenotypeVAE to improve the prediction performance of PerturbNet.

This disclosure also focuses on designing perturbations to the formation of desired cell states and discovering components of chemical and genetic perturbations that have important biological effects. The project was motivated by the problem of finding perturbations to shift some diseased cells to a healthy cell state. Some implementations formulate the objective to design an optimal perturbation to translate a group of cells to approximate a target cell state. Some implementations propose two optimal translation algorithms based on PerturbNet. Some implementations show that both continuous optimal translation and discrete optimal translation can find an optimal perturbation to translate a group of observed cells to approximate a group of target cells in their latent space. Some implementations also utilize a model interpretability method to interpret atomic scores of a drug treatment and G0 scores in forming a cell state. Some implementations also employ the model interpretability method to atomic scores of fitted optimal perturbations from the discrete optimal translation algorithm. In addition, some implementations interpret and identify G0 terms that have high attributions to distinguish a pair of genetic perturbations in their cell states.

### Example Methods

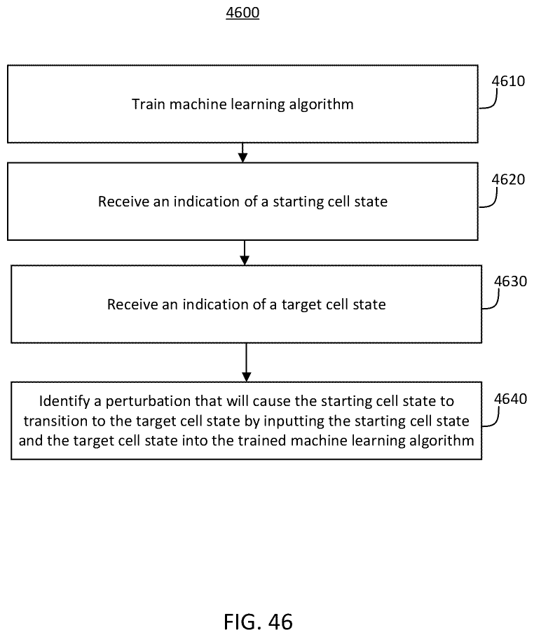

FIG. 46 illustrates an example method 4600 for identifying a perturbation. In some embodiments, the example method 4600 is performed by the perturbation computing device 102. It should be understood that the perturbations referred to in the following discussion may be chemical perturbations, genetic perturbations, etc.

The example method 4600 begins at block 4610 when a machine learning algorithm is trained (e.g., by the machine learning algorithm application 124, etc.).

In some embodiments, the training of the machine learning algorithm involves training three different networks. For example, a first network 310 may be trained to convert perturbations into real-valued vector representations of the perturbations; a second network 320 may be trained that converts cell states into real-valued vector representations of the cell states; and a third network 330 may be trained that maps relationships between: (i) the real-valued vector representations of the perturbations, and (ii) the real-valued vector representations of the cell states.

The networks may be any suitable type of network. In some examples, the first network 310 comprises a perturbation encoder; the second network 320 comprises a cell encoder; and the third 330 network comprises a conditional invertible neural network (cINN).

In some embodiments, the third network 330 is trained on high-throughput, single-cell screening data (e.g., data from a high-throughput chemical screen experiment).

Further, at any point in time, some of the networks may be swapped out for other networks. For example, the first network 310 may be a first perturbation network (e.g., a network that converts perturbations into real-valued vector representations of the perturbations), which may be swapped out for a second perturbation network. Advantageously, this may allow selection of a network that is specific to a type or class of perturbation(s).

At block 4620, the perturbation computing device 102 may receive an indication of a starting cell state. At block 4630, the perturbation computing device 102 may receive an indication of a target cell state. In some embodiments, the starting cell state is a diseased cell state, and the target cell state is a healthy cell state. In other embodiments, the starting cell state is a first healthy cell state, and the target cell state is a second healthy cell state.

At block 4640, the perturbation computing device 102 may identify a perturbation that will cause the starting cell state to transition to the target cell state by inputting the starting cell state and the target cell state into the trained machine learning algorithm.

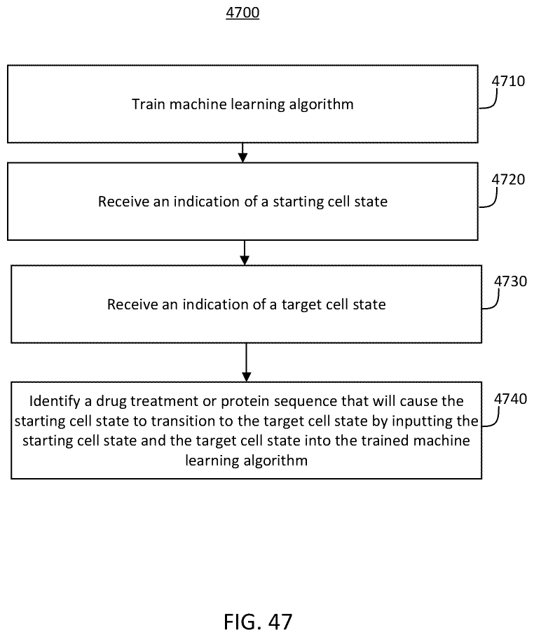

FIG. 47 illustrates an example method 4700 for identifying a drug treatment or protein sequence. In some embodiments, the example method 4700 is performed by the perturbation computing device 102.

The example method 4700 begins at block 4710 when a machine learning algorithm is trained (e.g., by the machine learning algorithm application 124, etc.).

In some embodiments, the training of the machine learning algorithm involves training three different networks. For example, a first network 310 may be trained to convert drug treatments or protein sequences into real-valued vector representations of the drug treatments or protein sequences; a second network 320 may be trained that converts cell states into real-valued vector representations of the cell states; and a third network 330 may be trained that maps relationships between: (i) the real-valued vector representations of the drug treatments or protein sequences, and (ii) the real-valued vector representations of the cell states.

The networks may be any suitable type of network. In some examples, the first network 310 comprises a drug treatment or protein sequence encoder; the second network 320 comprises a cell encoder; and the third 330 network comprises a conditional invertible neural network (cINN).

In some embodiments, the third network 330 is trained on high-throughput, single-cell screening data (e.g., data from a high-throughput chemical screen experiment).

Further, at any point in time, some of the networks may be swapped out for other networks. For example, the first network 310 may be a first drug treatment or protein sequence network (e.g., a network that converts drug treatments or protein sequences into real-valued vector representations of the drug treatments or protein sequences), which may be swapped out for a second drug treatment or protein sequence network. Advantageously, this may allow selection of a network that is specific to a type or class of drug treatment(s) or protein sequence(s).

At block 4720, the perturbation computing device 102 may receive an indication of a starting cell state. At block 4730, the perturbation computing device 102 may receive an indication of a target cell state. In some embodiments, the starting cell state is a diseased cell state, and the target cell state is a healthy cell state. In other embodiments, the starting cell state is a first healthy cell state, and the target cell state is a second healthy cell state.

At block 4740, the perturbation computing device 102 may identify a drug treatment or protein sequence that will cause the starting cell state to transition to the target cell state by inputting the starting cell state and the target cell state into the trained machine learning algorithm.

Further regarding the example flowcharts provided above, it should be noted that all blocks are not necessarily required to be performed. Moreover, additional blocks may be performed although they are not specifically illustrated in the example flowcharts. In addition, block(s) from one example flowchart may be performed in another of the example flowcharts.

### Additional Exemplary Embodiments

Aspect 1. A computer-implemented method for identifying a perturbation, the method comprising:
receiving, via one or more computer processors, an indication of a starting cell state;
receiving, via the one or more processors, an indication of a target cell state; and
identifying, via the one or more processors, a perturbation that will cause the starting cell state to transition to the target cell state by inputting the starting cell state and the target cell state into a trained machine learning algorithm.

Aspect 2. The computer-implemented method of aspect 1, wherein the perturbation is a chemical perturbation.

Aspect 3. The computer-implemented method of any one of aspects 1-2, wherein the perturbation is a genetic perturbation.

Aspect 4. The computer-implemented method of any one of aspects 1-3, wherein the trained machine learning algorithm comprises:
a first network that converts perturbations into real-valued vector representations of the perturbations;
a second network that converts cell states into real-valued vector representations of the cell states; and
a third network that maps relationships between: (i) the real-valued vector representations of the perturbations, and (ii) the real-valued vector representations of the cell states.

Aspect 5. The computer-implemented method of any one of aspects 1-4, wherein:
the trained machine learning algorithm comprises: (i) a first network, (ii) a second network, and (iii) a third network; and
the trained machine learning algorithm is trained by:
   training, via one or more processors, the first network, wherein the first network converts perturbations into real-valued vector representations of the perturbations;
   training, via the one or more processors, the second network, wherein the second network converts cell states into real-valued vector representations of the cell states; and
   training, via the one or more processors, the third network to learn relationships between: (i) real-valued vector representations of the perturbations, and (ii) the real-valued vector representations of the cell states.

Aspect 6. The computer-implemented method of any one of aspects 1-5, wherein the third network is a conditional invertible neural network (cINN).

Aspect 7. The computer-implemented method of any one of aspects 1-6, wherein the starting cell state is a diseased cell state, and the target cell state is a healthy cell state.

Aspect 8. The computer-implemented method of any one of aspects 1-7, wherein the starting cell state is a first healthy cell state, and the target cell state is a second healthy cell state.

Aspect 9. The computer-implemented method of any one of aspects 1-8, wherein the trained machine learning algorithm is trained on high-throughput, single-cell screening data.

Aspect 10. A computer system for identifying a perturbation, the computer system comprising one or more processors configured to:
receive an indication of a starting cell state;
receive an indication of a target cell state; and
identify a perturbation that will cause the starting cell state to transition to the target cell state by inputting the starting cell state and the target cell state into a trained machine learning algorithm.

Aspect 11. The computer system of aspect 10, wherein the perturbation is a chemical perturbation.

Aspect 12. The computer system of any one of aspects 10-11, wherein the perturbation is a genetic perturbation.

Aspect 13. The computer system of any one of aspects 10-12, wherein the trained machine learning algorithm comprises:
a first network that converts perturbations into real-valued vector representations of the perturbations;
a second network that converts cell states into real-valued vector representations of the cell states; and
a third network that maps relationships between: (i) the real-valued vector representations of the perturbations, and (ii) the real-valued vector representations of the cell states.

Aspect 14. The computer system of any one of aspects 10-13, wherein:
the trained machine learning algorithm comprises: (i) a first network, (ii) a second network, and (iii) a third network; and
the one or more processors are further configured to train the machine learning algorithm by:
   training the first network, wherein the first network converts perturbations into real-valued vector representations of the perturbations;
   training the second network, wherein the second network converts cell states into real-valued vector representations of the cell states; and
   training the third network to learn relationships between: (i) real-valued vector representations of the perturbations, and (ii) the real-valued vector representations of the cell states.

Aspect 15. The computer system of any one of aspects 10-14, wherein the starting cell state is a diseased cell state, and the target cell state is a healthy cell state.

Aspect 16. The computer system of any of aspects 10-15, wherein the starting cell state is a first healthy cell state, and the target cell state is a second healthy cell state.

Aspect 17. A computing device for identifying a perturbation, the computing device comprising:
one or more processors; and
one or more memories coupled to the one or more processors;
the one or more memories including computer executable instructions stored therein that, when executed by the one or more processors, cause the one or more processors to:
   receive an indication of a starting cell state;
   receive an indication of a target cell state; and
   identify a perturbation that will cause the starting cell state to transition to the target cell state by inputting the starting cell state and the target cell state into a trained machine learning algorithm.

Aspect 18. The computing device of aspect 17, wherein the perturbation is a chemical perturbation.

Aspect 19. The computing device of any one of aspects 17-18, wherein:
the trained machine learning algorithm comprises: (i) a first network, (ii) a second network, and (iii) a third network; and
the third network samples from a distribution of a cell state conditioned on a distribution of a perturbation representation.

Aspect 20. The computing device of any one of aspects 17-19, wherein:
the trained machine learning algorithm comprises: (i) a first network, (ii) a second network, and (iii) a third network; and
the one or more memories including computer executable instructions stored therein that, when executed by the one or more processors, further cause the one or more processors to train the machine learning algorithm by:
   training the first network, wherein the first network converts perturbations into real-valued vector representations of the perturbations;
   training the second network, wherein the second network converts cell states into real-valued vector representations of the cell states; and
   training the third network to learn relationships between: (i) real-valued vector representations of the perturbations, and (ii) the real-valued vector representations of the cell states.

### Other Matters

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Furthermore, the patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed:

1. A computer-implemented method for identifying a perturbation, the method comprising:
receiving, via one or more computer processors, an indication of a starting cell state;
receiving, via the one or more processors, an indication of a target cell state; and
identifying, via the one or more processors, a perturbation that will cause the starting cell state to transition to the target cell state by inputting the starting cell state and the target cell state into a trained machine learning algorithm.

2. The computer-implemented method of claim 1, wherein the perturbation is a chemical perturbation.

3. The computer-implemented method of claim 1, wherein the perturbation is a genetic perturbation.

4. The computer-implemented method of claim 1, wherein the trained machine learning algorithm comprises:
a first network that converts perturbations into real-valued vector representations of the perturbations;
a second network that converts cell states into real-valued vector representations of the cell states; and
a third network that maps relationships between: (i) the real-valued vector representations of the perturbations, and (ii) the real-valued vector representations of the cell states.

5. The computer-implemented method of claim 1, wherein:
the trained machine learning algorithm comprises: (i) a first network, (ii) a second network, and (iii) a third network; and
the trained machine learning algorithm is trained by:
training, via one or more processors, the first network, wherein the first network converts perturbations into real-valued vector representations of the perturbations;
training, via the one or more processors, the second network, wherein the second network converts cell states into real-valued vector representations of the cell states; and
training, via the one or more processors, the third network to learn relationships between: (i) real-valued vector representations of the perturbations, and (ii) the real-valued vector representations of the cell states.

6. The computer-implemented method of claim 5, wherein the third network is a conditional invertible neural network (cINN).

7. The computer-implemented method of claim 1, wherein the starting cell state is a diseased cell state, and the target cell state is a healthy cell state.

8. The computer-implemented method of claim 1, wherein the starting cell state is a first healthy cell state, and the target cell state is a second healthy cell state.

9. The computer-implemented method of claim 1, wherein the trained machine learning algorithm is trained on high-throughput, single-cell screening data.

10. A computer system for identifying a perturbation, the computer system comprising one or more processors configured to:
receive an indication of a starting cell state;
receive an indication of a target cell state; and
identify a perturbation that will cause the starting cell state to transition to the target cell state by inputting the starting cell state and the target cell state into a trained machine learning algorithm.

11. The computer system of claim 10, wherein the perturbation is a chemical perturbation.

12. The computer system of claim 10, wherein the perturbation is a genetic perturbation.

13. The computer system of claim 10, wherein the trained machine learning algorithm comprises:
a first network that converts perturbations into real-valued vector representations of the perturbations;
a second network that converts cell states into real-valued vector representations of the cell states; and
a third network that maps relationships between: (i) the real-valued vector representations of the perturbations, and (ii) the real-valued vector representations of the cell states.

14. The computer system of claim 10, wherein:
the trained machine learning algorithm comprises: (i) a first network, (ii) a second network, and (iii) a third network; and
the one or more processors are further configured to train the machine learning algorithm by:
training the first network, wherein the first network converts perturbations into real-valued vector representations of the perturbations;
training the second network, wherein the second network converts cell states into real-valued vector representations of the cell states; and
training the third network to learn relationships between: (i) real-valued vector representations of the perturbations, and (ii) the real-valued vector representations of the cell states.

15. The computer system of claim 10, wherein the starting cell state is a diseased cell state, and the target cell state is a healthy cell state.

16. The computer system of claim 10, wherein the starting cell state is a first healthy cell state, and the target cell state is a second healthy cell state.

17. A computing device for identifying a perturbation, the computing device comprising:
one or more processors; and
one or more memories coupled to the one or more processors;
the one or more memories including computer executable instructions stored therein that, when executed by the one or more processors, cause the one or more processors to:
receive an indication of a starting cell state;
receive an indication of a target cell state; and
identify a perturbation that will cause the starting cell state to transition to the target cell state by inputting the starting cell state and the target cell state into a trained machine learning algorithm.

18. The computing device of claim 17, wherein the perturbation is a chemical perturbation.

19. The computing device of claim 17, wherein:
the trained machine learning algorithm comprises: (i) a first network, (ii) a second network, and (iii) a third network; and
the third network samples from a distribution of a cell state conditioned on a distribution of a perturbation representation.

20. The computing device of claim 17, wherein:
the trained machine learning algorithm comprises: (i) a first network, (ii) a second network, and (iii) a third network; and
the one or more memories including computer executable instructions stored therein that, when executed by the one or more processors, further cause the one or more processors to train the machine learning algorithm by:
training the first network, wherein the first network converts perturbations into real-valued vector representations of the perturbations;
training the second network, wherein the second network converts cell states into real-valued vector representations of the cell states; and
training the third network to learn relationships between: (i) real-valued vector representations of the perturbations, and (ii) the real-valued vector representations of the cell states.
